(19) United States
(12) Patent Application Publication
(10) Pub. No.: US 2023/0295571 A1
(43) Pub. Date: Sep. 21, 2023

(54) COMPOSITIONS AND METHODS FOR STEM CELL CHONDROGENESIS

(71) Applicants: Washington University, St. Louis, MO (US); Shriners Hospitals for Children, Tampa, FL (US)

(72) Inventors: CHIA-LUNG WU, St. Louis, MO (US); AMANDA DICKS, St. Louis, MO (US); FARSHID GUILAK, St. Louis, MO (US)

(21) Appl. No.: 17/796,579

(22) PCT Filed: Jan. 29, 2021
(86) PCT No.: PCT/US21/15879

§ 371 Date: Jul. 29, 2022

Related U.S. Application Data
(60) Provisional application No. 62/967,257, filed on Jan. 29, 2020.

(51) Int. Cl. C12N 5/077; A61P 19/02

(52) U.S. Cl. CPC C12N 5/0655; A61P 19/02

(57) ABSTRACT

Among the various aspects of the present disclosure is the provision of compositions and methods for stem cell chondrogenesis. An aspect of the present disclosure provides for a method of differentiating pluripotent stem cells (e.g., induced pluripotent stem cells (iPSCs), human induced pluripotent stem cells (hiPSCs)) into chondrocyte-like cells (e.g., cartilage).

### CROSS REFERENCE TO RELATED APPLICATION

This application claims the benefit of U.S. Provisional Application 62/967,257, filed Jan. 29, 2020, the disclosure of which is hereby incorporated by reference in their entirety.

### GOVERNMENTAL RIGHTS

This invention was made with government support under AG015768 awarded by the National Institutes of Health. The government has certain rights in the invention.

### FIELD OF THE TECHNOLOGY

This disclosure relates to chondrocyte-like cells that are induced from pluripotent stem cells having properties of the chondrocytes and to processes for producing the chondrocyte-like cells. The invention also concerns cell preparations for cartilage tissue regeneration, implants, implant producing processes, cartilage disease therapeutic methods, and drug efficacy determining methods for determining the efficacy of a tested substance for cartilage disease, all using the chondrocyte-like cells.

### BACKGROUND

Articular cartilage has a role as a joint lubricant for absorbing impact at the diarthrodial joints during articular movement. The mechanical functions of the cartilage are imparted by the cartilage extracellular matrix constructed from type II and type XI collagens, and collagenous fibrils such as proteoglycan. It is known that the cartilage extracellular matrix is produced by the chondrocytes intrinsic to the cartilage.

Osteoarthritis is a typical cartilage tissue disease, caused by the aggravation of wear, damage, and degeneration of the articular cartilage in response to mechanical stresses (such as repetitive loading, excessive exercise, and trauma) and aging. The symptoms of osteoarthritis include joint pain during joint movement (movement pain) and a restricted range of motion (restricted motion), which lower the quality of daily life. In Japan, osteoarthritis affects about 20% of the population over the age 50 and is expected to affect more people as the medical development and improved lifestyle are expected to raise the average life expectancy. Osteoarthritis thus poses a big challenge in the aging society.

Conventional osteoarthritis therapies employ resting to prevent aggravation of symptoms or controlling pain by, for example, the administration of antiphlogistic analgetics or supplements or the intraarticular administration of joint lubricants. These methods, however, are only supportive and do not represent a definitive therapy, because the diseased chondrocytes have only weak repairing capabilities and cannot regenerate cartilage tissue. A procedure using a metallic artificial replacement joint has been practiced for osteoarthritis cases with progressive cartilage degeneration. However, artificial joints have a number of drawbacks, including a heavy burden put on patients during the procedure, deterioration due to wear, a tendency to dislocate, and possible revision surgery necessitated by a loosened artificial joint.

Recently, a technique that enables a definitive treatment of osteochondrosis deformans through cartilage tissue regeneration has caught attention for the treatment of osteochondrosis deformans which does not respond well to conventional therapies. For such a technique to be realized, development of an easy-to-obtain cell supply source that can produce large numbers of cells while retaining the capability to differentiate and form cartilage tissue is urgently needed. Chondrocytes are considered to be a good cell source candidate for cartilage tissue regeneration. However, because chondrocytes are limited in number and dedifferentiate through monolayer expansion, recent studies focus more on the development of a technique that induces formation of cartilage tissue with the use of bone marrow-derived mesenchymal stem (MS) cells or embryonic stem (ES) cells. However, MS cells have only limited proliferative capabilities, and recent studies suggest that the cartilage produced from MS cells is unstable and lacks sufficient cartilage properties. With regard to ES cell-derived differentiated cells, there are concerns that the cells, as an inhomogeneous population, may fail to provide sufficient cartilage tissue functions. Induced pluripotent stem (iPS) cells are an alternative; however, their use for cartilage tissue regeneration requires the establishment of a technique that enables the cells to differentiate into a homogeneous chondrocyte population, and there are still technical problems that need to be solved for practical applications in cartilage tissue regeneration.

Thus there remains a need for the development of cells that can be directly induced to only chondrocytes and that have cartilage tissue regenerative capabilities and a proliferative ability.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-1E show differentially expressed genes (DEGs) of mesodermal and chondrogenic differentiation of three human iPSC (hiPSC) lines by bulk RNA-seq. FIG. 1A is a schematic of the chondrogenic differentiation protocol for hiPSCs. FIG. 1B shows PCA indicates that three unique hiPSC lines followed similar differentiation trajectories in mesodermal lineage differentiation. FIG. 1C shows PCA indicates that three unique hiPSC lines followed similar differentiation trajectories in chondrogenesis. FIG. 1D shows DEGs averaged from three unique hiPSC lines at each stage of differentiation, respectively (mesodermal lineage differentiation). FIG. 1E shows DEGs averaged from three unique hiPSC lines at each stage of differentiation, respectively (chondrogenesis). Each column of the heatmap represents a comparison between two stages/time points, and each gene presented was assigned a colored dot (following the gene label). The color of the dot matches the color of the timepoint label on the left side of the heatmap. When the color of a gene label and a timepoint label match, that gene was significantly upregulated at the corresponding time points.

FIG. 2A-2E show in vitro and in vivo characterization of hiPSC-derived chondrocytes. FIG. 2A shows temporal gene expression of chondrogenic markers SOX9 and COL2A1, hypertrophic marker COL10A1, and osteogenic marker COL1A1. FIG. 2B shows that pellets showed enriched Saf-O, COL2A1, and COL6A1 staining. Most COL1A1 staining (green arrowheads) was located at the edge of the pellets, while faint COL10A1 (yellow arrowheads) was observed. Left column scale bar=400 μm. Right column scale bar=200 μm. Inset scale bar=50 μm. The experiment was repeated three times with similar results. FIG. 2C shows heatmap of 134 significantly upregulated genes identified in GO term cartilage development (GO:0051216). Genes in red font are either transcription factors or transcription regulators. FIG. 2D shows hiPSC-derived chondrocytes exhibit a similar phenotype to embryonic limb bud chondrocytes. FIG. 2E shows hiPSC-derived chondrocytes repaired osteochondral defects in the cartilage of mouse knee joints and retained a chondrocyte phenotype 28 days post implantation. n=3 mice per group. Top row scale bar=500 μm. Bottom row scale bar=100 μm.

FIG. 3A-3E show scRNA-seq and WGCNA reveal neural cells and melanocytes as off-target cells. FIG. 3A shows scRNA-seq was performed at hiPSC, Sclerotome, Cp, and six chondrogenic pellet time points. FIG. 3B shows reconstruction of differentiation trajectory revealing an off-target lineage bifurcation toward neural cells. A total of 19,195 cells, which passed quality control from the stage of hiPSC to d42 chondrogenic pellet, were used to reconstruct the differentiation trajectory. FIG. 3C shows chondrogenic markers were enriched in the chondrogenic branch, while neurogenic markers were observed in the branch of neurogenesis. FIG. 3D shows annotated cell populations at different time points during hiPSC chondrogenesis. Cells that passed quality are used for tSNE plots; Cp: 1888 cells, dl: 2216 cells, d7: 1200 cells, d14: 2148 cells, d28: 1271 cells, and d42: 1328 cells. FIG. 3E shows WGCNA and GO term analysis identified WNT4 as a hub gene of neurogenesis while WNT2B was highly associated with melanocyte development. scRNA-seq data of d14 pellets (with a total of 2148 cells and 3784 genes) was used for this computation.

FIG. 4A-4E show WNT inhibition during pellet culture enhanced homogeneity of hiPSC chondrogenesis. FIG. 4A shows the experimental scheme of WNT inhibition. FIG. 4B shows WNT-C59 treatment during pellet culture enhanced Saf-0 staining and decreased off-target cells (yellow arrowheads) as compared to other WNT inhibition culture regiments. Top row scale bar=400 μm. Bottom row scale bar=200 μm. The experiment was performed twice with similar results. FIG. 4C shows pellets treated with WNT-C59 in only pellet culture exhibited an increased GAG/DNA ratio compared to pellets treated with other culture regiments. *p=0.00001 at d28. #p=0.0228 at d42. Mean±SEM. n=4 pellets per group. Statistical significance was determined by one-way ANOVA with Tukey's post hoc test at a specific time point. FIG. 4D shows WNT-C59 significantly decreased, but WNT3A significantly increased, CD146+/CD166+/CD45− progenitors at the Cp stage. Different letters are significantly different (a vs. b, p=0.0005; a vs. c, p=0.0021; b vs. c, p=0.0001). Mean±SEM. n=3 per group (independent experiment). Statistical significance was determined by one-way ANOVA with Tukey's post hoc test. FIG. 4E shows RNA-FISH of d28 pellets showing WNT-C59-treated pellets had decreased WNT3A and WNT4 labeling (green) but more homogenous COL2A1 distribution (red) in the pellets. Scale bar=200 μm. The experiment was performed twice with similar results.

FIG. 5A-5G show scRNA-seq of pellets with WNT inhibition have improved chondrogenesis. FIG. 5A shows scRNA-seq was performed on the pellets with WNT inhibition. FIG. 5B shows chondrocytes and mesenchymal cells were two major populations in WNT-C59-treated pellets. Cells that passed quality control were used for tSNE plots; hiPSC: 4798 cells, Cp: 1888 cells, d7: 1682 cells, d14: 3076 cells, d28: 1756 cells, and d42: 1483 cells. FIG. 5C shows differentiation trajectory of WNT-C59-treated pellets. scRNA-seq data with a total of 14,683 cells from the stage of hiPSC, Cp, d7, d14, d28, and d42 WNT-C59-treated pellets were used to reconstruct the differentiation trajectory. FIG. 5D shows WNT-C59-treated pellets exhibited decreased neurogenic markers but increased chondrogenic markers. FIG. 5E shows multiple canonical correlation analysis (CCA) alignment of d7-d42 pellets. A total of 7977 cells from d7-d42 timepoints of WNT-C59-treated pellets were used to perform CCA alignment. FIG. 5F shows dynamic changes in gene expression and percentages of chondrocyte subpopulations over time. FIG. 5G shows a heat map of the top 20 DEGs at each timepoint for LECT1/EPYC/FRZB+ early mature chondrocytes.

FIG. 6A-6G show CCA analysis reveals that most WNTs, except WNT5B, were secreted by off-target cells. FIG. 6A shows three major conserved populations in d14 pellets. A total of 5224 cells from the d14 pellets with or without WNT-C59 treatment was analyzed. FIG. 6B shows violin plots of the specific markers for each conserved population. FIG. 6C shows WNT-C59-treated pellets comprised more chondrocytes and mesenchymal cells. FIG. 6D shows expression levels of chondrogenic markers were higher in WNT-C59-treated pellets while expression of neurogenic and melanocyte markers was higher in TGF-β3-treated pellets. FIG. 6E shows dot plot showing proliferative cells (mainly neural cells) from TGF-β3-treated pellets had high expression levels of WNT ligands. WNT inhibition largely decreased expression levels of WNTs in cells. FIG. 6F shows western blots confirm that WNT inhibition significantly decreased WNTs in cells at protein levels. *p=0.026, #p=0.021, $p=0.0003, †p=0.00029, ‡p=0.021 to its corresponding group. Mean±SEM. n=3 per treatment condition. Statistical significance was determined by a two-tailed Student's t test for the groups with or without specific WNT inhibition. FIG. 6G shows most WNTs were upregulated along the lineage of neural cells, where WNT5B was clustered with chondrogenic differentiation in TGF-β3-treated pellets. A total of 2148 cells from the TGF-β3-treated d14 pellets was analyzed and used to generate the heatmap.

FIG. 7A-7G show heterogenous multicellular WNT signaling models. FIG. 7A shows RT-qPCR of pellets treated with various WNTs during pellet culture. Different letters are significantly different from each other (p<0.05). Mean±SEM. n=3-4 pellets per group. Statistical significance was determined by one-way ANOVA with Tukey's post hoc test. FIG. 7B shows GAG/DNA ratios of pellets treated with various WNTs during pellet culture. Different letters are significantly different from each other (p<0.05). Mean±SEM. n=3-4 pellets per group. Statistical significance was determined by one-way ANOVA with Tukey's post hoc test. FIG. 7C shows WNT treatment increased infiltration of off-target cells (pink arrowheads and white dashed lines) into the pellets, decreased COL2A1 staining, and increased COL1A1 (yellow arrowheads) and COL10A1 staining in the pellets. The pellets with WNT-C59 treatment exhibited homogenous COL2A1 staining and decreased COL1A1 and COL10A1 staining. Scale bar=0.2 mm. The experiment was performed twice with similar results. FIG. 7D shows heatmap showing distinct expression levels of various WNTs in various cellular subpopulations in d14 TGF-β3-treated pellets. A total of 2148 cells from the TGF-β3-treated d14 pellets were analyzed and used to generate the heatmap. FIG. 7E shows the percentage of the cells expressing WNT3A and its putative receptors in d14 TGF-β3-treated pellets. FIG. 7F shows WNT3A-FZD2 heterogenous multicellular signaling models in d14 TGF-β3-treated pellets. FIG. 7G shows WNT5B-FZD1 and WNT2B-FZD4 heterogenous multicellular signaling models in d14 TGF-β3-treated pellets.

FIG. 8A-8C show step-wise differentiation of hiPSCs toward chondrocytes via specification of the paraxial mesoderm. FIG. 8A shows the differentiation protocol of hiPSCs into chondrocytes. FIG. 8B shows cell morphology at each stage during mesodermal differentiation. Please note that low cell density at hiPSC stage is required to obtain successful mesodermal differentiation. Scale bar=500 μm. FIG. 8C shows up-regulation of stage-specific markers for 3 unique hiPSC lines.

FIG. 9A-9D show GO enrichment analysis of bulk RNA-seq data and subcutaneous implantation of hiPSC-derived chondrocytes in mice. FIG. 9A shows GO enrichment analysis of bulk RNA-seq data showing that up-regulated genes were involved in skeletal system and cartilage development. FIG. 9B shows d14 chondrogenic pellets maintained a cartilage phenotype indicated by intense Saf-O and COL2A1 staining after 14 days of subcutaneous implantation in mice. n=3 mice. FIG. 9C shows the off-target cells (mostly located at the edge of perichondrium, yellow arrowheads) were observed in the pellets derived from 3 distinct hiPSC lines. FIG. 9D shows focal black dots were occasionally observed on the surface of the pellets.

FIG. 10A-10D show analysis of scRNA-seq data reveals diverse cell populations in hiPSC-derived chondrogenic pellets. FIG. 10A shows scRNA-seq of mixed specie samples showing low multiplet rates (<2.7%). FIG. 10B shows CCA of scRNA-seq data from d28 chondrogenic pellets from 2 independent experiments (i.e., 2 batches). 8 conserved cell clusters were identified in both batches.

FIG. 10C shows cells in the same cluster from different batches exhibited high correlation in their gene expression (Spearman's rank coefficient rₛ>0.87 for all clusters). FIG. 10D shows cells in the clusters from distinct batches demonstrated similar gene expression patterns. FIG. 10E shows additional neural cell markers such as DCX, MAP2, OTX1, and PAX6 were also enriched in the branch of neurogenic differentiation. FIG. 10F shows SOX4+ and SOX4/SOX9+ cells at the Cp stage had high expression of neural crest cell markers. A total of 1,888 cells at the Cp stage that passed quality control were analyzed. FIG. 10G shows cells that are enriched for PRRX1, COL1A1, COL3A1, and COL5A1 were annotated as “mesenchyme” at the Cp stage. A total of 1,888 cells at the Cp stage that passed quality control were analyzed. FIG. 10H shows three major cell populations observed in d3 pellets. A total of 2,485 cells from d3 pellets that passed quality control were used to generate the tSNE plot. FIG. 10I shows a fraction of major cell types over the course of differentiation (Cp—d28). A total of 11,208 cells from the Cp stage to d28 pellets were analyzed. FIG. 10J shows IHC against nestin and MITF confirms the presence of neural cells and melanocytes in pellets. FIG. 10K shows mesenchymal cells in d14 pellets expressed several conventionally recognized MSC markers. However, whether these mesenchymal cells exhibit multipotency like MSCs requires further investigation. A total of 2,148 cells from d14 pellets were analyzed.

FIG. 11A-11I show WGCNA reconstructed gene regulatory networks (GRNs) of neurogenesis and melanogenesis and identified the hub genes in each network. FIG. 11A shows GRNs of neurogenesis. Topological analysis (community cluster) was performed to visualize subnetworks. FIG. 11B shows GRNs of melanogenesis. Topological analysis (community cluster) was performed to visualize subnetworks. FIG. 11C shows WNT4 was among the hub genes in the GRN of neurogenesis while WNT2B was associated with the GRN of melanocyte development. FIG. 11D shows representative d28 pellet images showing that WNT-C59 or a combination of WNT-C59 and ML329 treatment during pellet culture enhanced the homogeneity of chondrogenesis by removing off-target cells. This was validated in 3 unique hiPSC lines. FIG. 11E shows representative d28 pellet images showing that WNT-C59 or a combination of WNT-C59 and ML329 treatment during pellet culture enhanced the homogeneity of chondrogenesis by removing off-target cells. This was validated in 3 unique hiPSC lines. FIG. 11F shows representative d28 pellet images showing that WNT-C59 or a combination of WNT-C59 and ML329 treatment during pellet culture enhanced the homogeneity of chondrogenesis by removing off-target cells. This was validated in 3 unique hiPSC lines. FIG. 11G shows the pellets treated with WNT-C59 or a combination of WNT-C59 and ML329 treatment exhibited significantly increased GAG/DNA ratios compared to the pellets treated with ML329 and the pellets treated TGF-β3. * WNT-C59 vs. TGF-β3 (p=0.01) at a specific timepoint. #WNT-C59+ML329 vs. TGF-β3 (p=0.001) at a specific timepoint. Mean±SEM. n=4 pellets per treatment condition. One-way ANOVA with Fisher's LSD was performed at d28 and d42. FIG. 11H shows hMSCs harvested from 3 distinct donors exhibited increased chondrogenesis when treated with WNT-C59 during pellet culture. FIG. 11I shows hMSCs harvested from donor 1 and donor 3 had significantly increased GAG/DNA ratios when treated with WNT-C59 compared to with TGF-β3 alone. #WNT-C59 vs. TGF-β3 (p=0.01) at specific time point. Mean±SEM. n=4 pellets per treatment condition. Two-tailed Student's t-test was performed at d28 and d42.

FIG. 12 shows semi-quantification of RNA-FISH against WNTs and COL2A1. WNT-C59-treated pellets showed decreased WNT3A and WNT4 expression but increased COL2A1 RNA-FISH labeling versus TGF-β3-treated pellets.

FIG. 13A-13J show multiple CCA alignment of d7-d42 pellets reveals that 4 conserved chondrocyte subpopulations and 1 conserved mesenchymal population were observed in WNT-C59-treated pellets. FIG. 13A shows Jitter plots showing that WNT-C59-treated pellets had increased expression of ACAN, COL2A1, and SOX9 but decreased SOX2 versus standard TGF-β3-treated pellets. FIG. 13B shows temporal expression profiles of signature genes of each chondrocyte subpopulation. CDK1 and IGFBP5 showed transient upregulation while COL9A1 and COL11A1 remained up-regulated once activated. MMP13 and MX1 showed increased expression levels at later time points. FIG. 13C shows dynamic changes in the percentage of the cell population within the pellets over the course of differentiation. FIG. 13D shows ISG15/IFI6/MX1+ chondrocytes contained 4.6% cells expressing both VEGFA and MMP13. FIG. 13E shows BMPR1B/ITGA4+ progenitors previously identified in articular cartilage were mostly observed in HMGB2/CDK1+ proliferating chondrocytes. FIG. 13F shows BMPR1B/ITGA4+ progenitors previously identified in articular cartilage were mostly observed in HMGB2/CDK1+ proliferating chondrocytes. FIG. 13G shows LECT1/EPYC/FRZB+ early-mature chondrocytes had the highest levels of COL2A1 and ACAN expression among other chondrocyte subpopulations. FIG. 13H shows ISG15/IFI16/MX1+ mature-hypertrophic chondrocytes expressed several IFN-related genes. FIG. 13I shows in comparison with IGFBP5+ early chondrocytes, ISG15/IFI6/MX1+ mature-hypertrophic chondrocytes showed high expression in IGFBP3 but decreased expression in FOS. FIG. 13J shows the expression of various hypertrophic chondrocyte markers. For scRNA-seq analysis of WNT-C59 treated pellets, a total 7,997 cells (from d7-d42) passed quality control and thus were analyzed for this figure. A total of 7,977 cells from d7-d42 timepoints of WNT-C59-treated pellets were used to performed CCA alignment.

FIG. 14A-14B show ACTA2/PRRX1/COL1A1+ mesenchymal cells in the pellets, but not mesenchymal cells at the Cp stage, exhibit similar gene expression profile to perichondrial cells. FIG. 13A shows ACTA2/PRRX1/COL1A1+ mesenchymal cells in the pellets expressed markers of rat perichondrial cells. FIG. 13B shows ACTA2/PRRX1/COL1A1+ mesenchymal cells from d7 and d14 pellets were enriched with 8 of 15 differentially expressed genes in the perichondrium-like membrane of the human chondrogenic pellet. Particularly, d7 ACTA2/PRRX1/COL1A1+ mesenchymal cells had the highest expression of C2orf91, FGF18, GGT7, CHST9, and ZNH354C. Interestingly, we also observed that there was a gradual shift in the gene expression profile of ACTA2/PRRX1/COL1A1+ mesenchymal cells from d28 to d42. For example, d28 ACTA2/PRRX1/COL1A1+ mesenchymal cells were enriched in NRN1 and CH3L1 while d42 cells had the highest expression of ADAMTSL1, WISP2, and CD70.

FIG. 15A-15E show the GRN of hiPSC chondrogenesis. FIG. 15A shows the GRN and hub genes of hiPSC chondrogenesis. FIG. 15B shows CCA was used to identify DEGs of each subpopulation between d14 pellets with and without C59 treatment. ID2, a neurogenic marker (blue circle), was decreased in proliferative cells in WNT-C59-treated pellets, while PRG4 (red circle) was increased in mesenchymal cells in WNT-C59-treated pellets. FIG. 15C shows CCA alignment of cells from d28 pellets with and without C59 treatment. A total of 3,027 cells from d28 pellets with and without WNT-C59 treatment were used to performed CCA alignment. FIG. 15D shows CCA was used to identify DEGs of chondrocytes between d28 pellets with and without WNT-C59 treatment. Markers for mature-hypertrophic chondrocytes, such as IFI6 and ISG15 (blue circles), were decreased while ACAN and COMP (red circles) were increased in WNT-C59-treated pellets. FIG. 15E shows that similar to the WNT expression profiles in d14 pellets, most WNTs were expressed by proliferative cells in the d28 pellets treated with TGF-β3.

FIG. 16A-16D show WNT treatment during chondrogenesis. FIG. 16A shows a schematic of WNT treatment during chondrogenic pellet culture. FIG. 16B shows RT-qPCR of d14 pellets treated various WNTs showing that gene expression of WNTs can be modulated by other WNT ligands. Different letters are significantly different from each other (p<0.05). Mean±SEM. n=3-4 pellets per group. Statistical significance was determined by one-way ANOVA with Tukey's post-hoc test. FIG. 16C shows semi-quantification of Saf-O and IHC labeling against various collagens. FIG. 16D shows percentage of the cells expressing a variety of WNTs in d14 pellets treated with TGF-β3. For scRNAseq analysis of d14 TGF-β3 treated pellets, a total of 2,148 cells passed quality control and thus were analyzed.

FIG. 17A-17E show differential expression of BMPs/GDFs and their receptors in response to WNT inhibition. FIG. 17A shows CCA alignment of chondrocyte and mesenchymal populations from TGF-β3 only and WNT-C59 conditions. FIG. 17B shows WNT-C59-treated pellets had a decreased percentage of BMP4-expressing cells within all clusters except within ISG15/IFI6/MX1+ mature-hypertrophic chondrocytes. FIG. 17C shows WNT-C59-treated pellets demonstrated a remarkably increased percentage of GDF5 and BMPR1B expressing cells within all clusters versus TGF-β3-treated pellets. FIG. 17D shows WNT-C59-treated pellets demonstrated a remarkably increased percentage of GDF5- and BMPR1B-expressing cells within all clusters versus TGF-β3-treated pellets. FIG. 17E shows WNT-C59 treatment decreased the percentage of cells expressing BMPR2 in LECT1/EPYC/FRZB+ early mature chondrocytes, ISG15/IFI16/MX1+ mature-hypertrophic chondrocytes, BJIP3/FAM162+ apoptotic chondrocytes, and HMGB2/CDK1+ and UBE2C/CCNB1/KPNA2+ proliferating chondrocytes. (B-E) Note that WNT-C59 treatment did not significantly affect the contribution of a cluster to the cells expressing BMP4, GDF5, BMPR1B, and BMPR2 as presented in the pie charts. For bioinformatic analysis, CCA was performed with a total of 1,335 cells from mesenchymal and chondrocyte populations from d14 TGF-β3 pellets and with a total of 3,047 cells from mesenchymal and chondrocyte populations from d14 WNT-C59 pellets.

FIG. 18A-18B show CCA analysis showing differential gene expression with WNT-C59 treatment. FIG. 18A shows BMP families in chondrocyte subpopulations due to WNT-C59 treatment. Numerical value on top of each bar in the bar graph indicates cell numbers expressing a given gene. For bioinformatics analysis, CCA was performed with a total of 1,335 cells from mesenchymal and chondrocyte populations from d14 TGF-β3 pellets and with a total of 3,047 cells from mesenchymal and chondrocyte populations from d14 WNT-C59 pellets. FIG. 18B shows GDF families in chondrocyte subpopulations due to WNT-C59 treatment.

FIG. 19A-19B show CCA analysis showing differential receptor gene expression with WNT-C59 treatment. FIG. 19A shows type I receptors for the BMP/GDF family in chondrocyte subpopulations due to WNT-C59 treatment. The numerical value on top of each bar in the bar graph indicates the number of cells expressing a given gene. For bioinformatic analysis, a total of 2,148 cells from d14 TGF-β3 treated pellets and total 3,076 cells from d14 WNT-C59+TGF-β3 treated pellets passed quality control and thus were analyzed for this figure. FIG. 19B shows type II receptors for the BMP/GDF family in chondrocyte subpopulations due to WNT-C59 treatment.

FIG. 20A-20B show the top 10 up-regulated genes. FIG. 20A shows the top 10 up-regulated genes in fold change in the mesodermal phase. FIG. 20B shows the top 10 up-regulated genes in fold change in the chondrogenic phase.

### DETAILED DESCRIPTION

The generation of the chondrocytes from human pluripotent stem cells (hPSCs) is a major goal for regenerative medicine. Osteoarthritis (OA) is a debilitating joint disease characterized by cartilage degeneration and pathologic remodeling of other joint tissues. Cartilage has limited intrinsic healing capacity, motivating the application of stem cells for regenerative therapies. In this regard, the advent of human induced pluripotent stem cells (hiPSCs) has served as a major breakthrough toward cartilage regenerative therapies and in vitro disease modeling for OA drug discovery. However, the development of protocols to consistently differentiate hiPSCs into chondrocytes remains challenging. Early studies reported that chondrocytes can be generated from hiPSCs via embryoid body formation followed by monolayer expansion of mesodermal cells and three-dimensional cell pellet culture in chondrogenic-induction medium. Despite some success, this approach was proven difficult to reproduce across different iPSC lines, potentially due to variability in lots of fetal bovine serum (FBS) generally used for cell expansion. Thus, recent strategies have sought to use serum-free and chemically defined medium. By coupling inductive and repressive signals required for mesoderm specification in embryonic development, applicants established a step-wise hiPSC chondrogenic differentiation protocol that was validated with multiple hiPSC lines.

An important consideration in the differentiation process of hiPSCs is that they are considered to be in a primed pluripotent state with increased genome-wide DNA methylation compared to ground state naïve pluripotent cells, such as preimplantation blastocysts. Therefore, even directed differentiation of hiPSCs can lead to the unpredictable formation of off-target cell populations. However, the gene regulatory networks (GRNs) leading to on- or off-target differentiation of hiPSCs, as well as the effect of the undesired cells on hiPSC chondrogenesis (i.e., heterocellular signaling), remain to be elucidated, particularly at the single-cell level.

The present disclosure is based, at least in part, on the use of bulk RNA sequencing (bulk RNA-seq) and single-cell RNA sequencing (scRNA-seq) throughout the process of mesodermal and chondrogenic differentiation of hiPSCs to map the dynamics of gene expression. By exploiting single-cell transcriptomics, the present disclosure confirmed the mesodermal and chondrogenic differentiation of hiPSCs in addition to identifying the GRNs and critical hub genes regulating the generation of heterogenous off-target cells. The present disclosure provides methods with significantly improved homogeneity of hiPSC chondrogenesis by inhibiting the molecular targets WNTs and MITF. Thus, the present disclosure provides an enhanced hiPSC chondrogenic differentiation protocol. As described herein, the present disclosure provides chondrocyte-like cells that are induced from pluripotent stem cells having properties of the chondrocytes. The disclosure also concerns cell preparations for cartilage tissue regeneration, implants, implant producing processes, cartilage disease therapeutic methods, and drug efficacy determining methods for determining the efficacy of a tested substance for cartilage disease, all using the chondrocyte-like cells.

Additional aspects of the disclosure are described below.

### (I) Methods of Producing Chondrocytes and Chondrocyte-Like Cells

Aspects described herein stem from, at least in part, development of methods that efficiently direct differentiation of pluripotent stem (PS) cells into chondrocyte and/or chondrocyte-like cells. As used herein, “chondrocyte-like cells” means cells that have a proliferative ability and the properties of the chondrocytes, with the capabilities to form or regenerate cartilage tissue (in other words, cartilage stem cells). Herein, “having the properties of the chondrocytes” means showing positive with the specific staining for chondrocytes and expressing chondrocyte marker genes. In particular, the present disclosure provides, inter alia, an in vitro or ex vivo culturing process for producing a population of chondrocyte and/or chondrocyte-like cells in a stage-specific manner preventing off-target cells and chondrocyte hypertrophy. Further, the chondrocyte and/or chondrocyte-like cells are transcriptionally and functionally similar to primary chondrocytes, including cartilage matrix deposition potential. In some embodiments, this culturing process may involve multiple differentiation stages (e.g., 2, 3, or more). Alternatively, or in addition, the culturing process may involve culture of the cells in the presence of a compound which enhances BMP signaling, inhibit WNT and/or melanocyte-inducing transcription factor (MITF) signaling. In some embodiment, the total time period for the in vitro or ex vivo culturing process described herein can range from about 26-68 days (e.g., 32-42 days, 35-45 days, or 40-60 days). In one example, the total time period is about 42 days.

As noted above, in some embodiments, the methods for producing chondrocyte and/or chondrocyte-like cells as disclosed herein may include multiple differentiation stages (e.g., 2, 3, 4, or more). For example, in non-limiting examples, the methods may include an anterior primitive streak differentiation step, e.g., the culturing of the pluripotent stem cells under differentiation conditions to obtain cells of the anterior primitive streak, a paraxial mesoderm differentiation step, e.g., the culturing of the obtained anterior primitive streak under differentiation conditions to obtain the paraxial mesoderm cells, an early somite differentiation step, e.g., the culturing of the obtained paraxial mesoderm cells under differentiation conditions to obtain the early somite cells, a sclerotome differentiation step, e.g., the culturing of the obtained early somite cells under differentiation conditions to obtain the sclerotome cells, a chondroprogenitor step, e.g., the culturing of the obtained sclerotome cells under differentiation conditions to obtain the chondroprogenitor cells and, a chondrogenic step, e.g., the culturing of the obtained chondroprogenitor cells under differentiation conditions to obtain the chondrocyte and/or chondrocyte-like cells.

Existing methods for producing human chondrocytes often result in low yield of chondrocytes accompanied by unpredictable and heterogeneous off-target differentiation of cells during chondrogenesis. The generation of chondrocytes from hiPSCs is a goal for both regenerative medicine and private industry scientists. However, to ensure that these chondrocytes faithfully recapitulate the functional behavior(s) of those found in humans, the presently disclosed hiPSC-derived chondrocytes have been derived from the developmental programs which identifying the gene regulatory networks and critical hub genes regulating the generation of heterogenous off-target cells. The in vitro or ex vivo model described herein can provide a reliable source of chondrocyte and/or chondrocyte-like cells. The pluripotent stem (PS) cell-derived chondrocytes and/or chondrocyte-like cells can be used in various applications, including, but not limited to, as an in vitro model, related diseases or disorders, drug discovery and/or developments, and cartilage tissue engineering.

Accordingly, embodiments of various aspects described herein relate to methods for generation of chondrocyte and/or chondrocyte-like cells from PS cells, cells produced by the same, and methods of use.

### (a) Pluripotent Stem Cells

In some embodiments, the in vitro or ex vivo culturing system disclosed herein may use pluripotent stem cells (e.g., human induced pluripotent stem cells) as the starting material for producing progenitor cells for various lineages. As used herein, “pluripotent” or “pluripotency” refers to the potential to form all types of specialized cells of the three germ layers (endoderm, mesoderm, and ectoderm) and is to be distinguished from “totipotent” or “totipotency”, which is the ability to form a complete embryo capable of giving rise to offspring. As used herein, “human pluripotent stem cells” (hPSCs) refers to human cells that have the capacity, under appropriate conditions, to self-renew as well as the ability to form any type of specialized cells of the three germ layers (endoderm, mesoderm, and ectoderm). hPS cells may have the ability to form a teratoma in 8-12-week old SCID mice and/or the ability to form identifiable cells of all three germ layers in tissue culture. Included in the definition of human pluripotent stem cells are embryonic cells of various types including human embryonic stem (hES) cells, [see, e.g., Thomson et al. (1998), Heins et. al. (2004)] and induced pluripotent stem cells [see, e.g. Takahashi et al., (2007); Zhou et al. (2009); Yu and Thomson in Essentials of Stem Cell Biology (2nd Edition)]. The various methods described herein may utilize hPS cells from a variety of sources. For example, hPS cells suitable for use may have been obtained from developing embryos by use of a nondestructive technique such as by employing the single blastomere removal technique described in Chung et al (2008), further described by Mercader et al. in Essential Stem Cell Methods (First Edition, 2009). Additionally or alternatively, suitable hPS cells may be obtained from established cell lines or may be adult stem cells.

In some aspects, the pluripotent stem cells for use according to the disclosure may be human embryonic stem cells. Various techniques for obtaining hES cells are known to those skilled in the art. In some instances, the hES cells for use according to the present disclosure are ones, which have been derived (or obtained) without destruction of the human embryo, such as by employing the single blastomere removal technique known in the art. See, e.g., Chung et al., Cell Stem Cell, 2(2):113-117 (2008), Mercader et al., Essential Stem Cell Methods (First Edition, 2009). Suitable hES cell lines can also be used in the methods disclosed herein. Examples include, but are not limited to, cell lines H1, H9, SA167, SA181, SA461 (Cellartis AB, Goteborg, Sweden) which are listed in the NIH stem cell registry, the UK Stem Cell bank, and the European hESC registry and are available on request. Other suitable cell lines for use include those established by Klimanskaya et al., Nature 444:481-485 (2006), such as cell lines MA01 and MA09, and Chung et al., Cell Stem Cell, 2(2):113-117 (2008), such as cell lines MA126, MA127, MA128 and MA129, which all are listed with the International Stem Cell Registry (assigned to Advanced Cell Technology, Inc. Worcester, MA, USA).

Alternatively, the pluripotent stem cells for use in the methods disclosed herein may be induced pluripotent stem cells (iPS) cells such as human iPS cells. As used herein “hiPS cells” refers to human induced pluripotent stem cells. hiPS cells are a type of pluripotent stem cells derived from non-pluripotent cells—typically adult somatic cells—by induction of the expression of genes associated with pluripotency, such as SSEA-3, SSEA-4, TRA-1-60, TRA-1-81, Oct-4, Sox2, Nanog and Lin28. Various techniques for obtaining such iPS cells have been established and all can be used in the present disclosure. See, e.g., Takahashi et al., Cell 131(5):861-872 (2007); Zhou et al., Cell Stem Cell. 4(5):381-384 (2009); Yu and Thomson in Essentials of Stem Cell Biology (2nd Edition, Chapter 4). It is also envisaged that the hematopoietic progenitor cells may also be derived from other pluripotent stem cells such as adult stem cells, cancer stem cells or from other embryonic, fetal, juvenile, or adult sources.

In some embodiments, human pluripotent stem cells, (wherein hPS cells can comprise both human embryonic stem cells (hES) cells and human induced pluripotent stem cells (hiPS) cells) can be cultured until about 70% confluence. These cells can be removed from these conditions, dissociated into clumps (termed “embryoid bodies”), and then further cultured under hypoxic conditions (about 1-5% O₂, 5% CO₂) in defined serum-free differentiation media.

In some embodiments, ES cell culture may be grown on one layer of feeder cells. “Feeder cells” refer to a type of cell, which can be second species, when being co-cultured with another type of cell. Feeder cells are generally derived from embryo tissue or tire tissue fibroblast. Embryo is collected from the CF1 mouse of pregnancy 13 days, is transferred in 2 ml trypsase/EDTA, then careful chopping, 37 DEG C incubate 5 minutes. 10% FBS is added, so that fragment is precipitated, cell increases in 90% DMEM, 10% FBS, and 2 mM glutamine. The feeder cells offer a growing environment for the ES cells. Certain form of ES cells can use, for example, primary mouse embryonic fibroblast or infinite multiplication mouse embryonic fibroblasts. In order to prepare feeder layer, irradiated cells may be used to support the ES cells (about 3000 rad γ-radiation will inhibit proliferation).

In some embodiments, the PS cells are removed from the feeder cells and cultured in serum free defined media for about 24 hours to generate embryoid bodies. Term “embryoid” is synonymous with “aggregation,” refers to differentiated and neoblast aggregation, which appears in ES cells. It is maintained in undue growth or the culture that suspends in monolayer cultures. Embryoid is different cell types (generally originating from different germinal layers) mixture, can according to morphological criteria distinguish and available immunocytochemistry detect cell marking. In some embodiments, the PS cells are cultured in a cell culture vessel coated with at least one extracellular matrix protein (e.g., laminin or Matrigel) to generate embryoid bodies.

In a preferred embodiment, the PS cells are grown in a monolayer to about 40% confluence.

### (b) Differentiation of Pluripotent Stem Cells

The in vitro or ex vivo culturing system disclosed herein may involve a step of differentiation to differentiate any of the PS cells disclosed herein to chondrocyte and/or chondrocyte-like cells.

Suitable conditions for mesoderm differentiation are known in the art (e.g., Sturgeon et al., Nat Biotechnol.; 32(6):554-61 (2014)) and/or disclosed in Examples below. As used herein “mesoderm” and “mesoderm cells (ME cells)” refers to cells exhibiting protein and/or gene expression as well as morphology typical to cells of the mesoderm or a composition comprising a significant number of cells resembling the cells of the mesoderm. The mesoderm is one of the three germinal layers that appears in the third week of embryonic development. It is formed through a process called gastrulation. There are three important components, the paraxial mesoderm, the intermediate mesoderm, and the lateral plate mesoderm. The paraxial mesoderm forms the somitomeres, which give rise to mesenchyme of the head and organize into somites in occipital and caudal segments, and give rise to sclerotomes (cartilage and bone), and dermatomes (subcutaneous tissue of the skin). Signals for somite differentiation are derived from surroundings structures, including the notochord, neural tube, and epidermis. The intermediate mesoderm connects the paraxial mesoderm with the lateral plate, eventually it differentiates into urogenital structures consisting of the kidneys, gonads, their associated ducts, and the adrenal glands. The lateral plate mesoderm give rise to the heart, blood vessels, and blood cells of the circulatory system as well as to the mesodermal components of the limbs.

Some of the mesoderm derivatives include the muscle (smooth, cardiac, and skeletal), the muscles of the tongue (occipital somites), the pharyngeal arches muscle (muscles of mastication, muscles of facial expressions), connective tissue, dermis, and subcutaneous layer of the skin, bone and cartilage, dura mater, endothelium of blood vessels, red blood cells, white blood cells, microglia, the kidneys, and the adrenal cortex.

Generally, in order to obtain ME cells, PS cells such as hPS cells can be cultured in a mesodermal differentiation medium comprising a defined lipid concentrate, insulin, selenous acid, monothioglycerol, antibiotics, and a differentiation inducer such as transferrin. The mesodermal differentiation medium may be optionally further supplemented with one or more growth factors, such as a fibroblast growth factor (FGF) (e.g., FGF1, FGF2 and FGF4), and one or more bone morphogenic proteins (BMP), such as BMP2 and BMP4. As used herein, the term “FGF” means fibroblast growth factor, preferably of human and/or recombinant origin, and subtypes belonging thereto are e.g. “bFGF” (means basic fibroblast growth factor, sometimes also referred to as FGF2) and FGF4. “aFGF” means acidic fibroblast growth factor (sometimes also referred to as FGF1). As used herein, the term “BMP” means Bone Morphogenic Protein, preferably of human and/or recombinant origin, and subtypes belonging thereto are e.g. BMP4 and BMP2. The concentration of the one or more growth factors may vary depending on the particular compound used. The concentration of FGF2, for example, is usually in the range of about 2 to about 50 ng/ml, such as about 2 to about 20 ng/ml. FGF2 may, for example, be present in the specification medium at a concentration of about 20 ng/ml. The concentration of FGF1, for example, is usually in the range of about 50 to about 200 ng/ml, such as about 80 to about 120 ng/ml. FGF1 may, for example, be present in the specification medium at a concentration of about 100 ng/ml. The concentration of FGF4, for example, is usually in the range of about 20 to about 40 ng/ml. FGF4 may, for example, be present in the specification medium at a concentration of about 30 ng/ml. The concentration of the one or more BMPs, is usually in the range of about 10 to about 300 ng/ml, such as about 50 to about 250 ng/ml, about 100 to about 250 ng/ml, about 150 to about 250 ng/ml, about 50 to about 200 ng/ml, about 100 to about 200 ng/ml, or about 150 to about 200 ng/ml. The concentration of BMP2, for example, is usually in the range of about 2 to about 50 ng/ml, such as about 10 to about 30 ng/ml. BMP2 may, for example, be present in the hepatic specification medium at a concentration of about 20 ng/ml.

In some embodiments, the mesodermal differentiation media comprises an activin, such as activin A or B. The concentration of activin is usually in the range of about 50 to about 200 ng/ml, such as about 80 to about 120 ng/ml. Activin may, for example, be present in the mesodermal differentiation medium at a concentration of about 90 ng/ml or about 100 ng/ml. As used herein, the term “Activin” is intended to mean a TGF-β family member that exhibits a wide range of biological activities including regulation of cellular proliferation and differentiation such as “Activin A” or “Activin B”. Activin belongs to the common TGF-β superfamily of ligands. The differentiation medium may further comprise an inhibitor of the activin receptor-like kinase receptors, ALK5, ALK4 and ALK7, such as SB431542 or SB505124. The concentration of the ALK5, ALK4 and ALK7 inhibitor is usually in the concentration of about 1 μM to about 12 μM, such as about 3 μM to about 9 μM. The mesodermal differentiation media may comprise a GSKβ-inhibitor, such as, e.g., CHIR99021 or CHIR98014. The concentration of the GSKβ inhibitor, if present, is usually in the range of about 0.1 to about 10 μM, such as about 0.05 to about 5 μM.

In some embodiments, the mesodermal differentiation media comprises an ATP-competitive inhibitor of AMPK (AMP-activated protein kinase) and/or a selective inhibitor of bone morphogenetic protein (BMP) signaling. In some embodiments, the ATP-competitive inhibitor of AMPK (AMP-activated protein kinase) and/or a selective inhibitor of bone morphogenetic protein (BMP) signaling is dorsomorphin. The concentration of the dorosmorphin is usually in the concentration of about 1 μM to about 12 μM, such as about 3 μM to about 9 μM.

In some embodiments, the mesodermal differentiation media comprises WNT signaling inhibitor. Wnt signaling pathways are a group of signal transduction pathways made of proteins and play an important role in passing signals from outside of a cell through cell surface receptors to the inside of the cell. Wnt inhibitors belong to small protein families, including sFRP, Dkk, WIF, Wise/SOST, Cerberus, IGFBP, Shisa, Waif1, APCDD1, and Tiki1. Their common feature is to antagonize Wnt signaling by preventing ligand-receptor interactions or Wnt receptor maturation. WNT-C59 is a highly potent inhibitor of Porcupine (PORCN), a membrane-bound O-acyltransferase (MBOAT) (IC₅₀=74 μM) shown to inhibit Wnt signaling pathways. In some embodiments, the WNT inhibitor is WNT-C59. The concentration of the WNT-C59 is usually in the concentration of about 0.01 μM to about 4 μM, such as about 0.3 μM to about 3 μM.

In some embodiments, the mesodermal differentiation media comprises FGFR and/or VEGFR inhibitor. PD173074 is a potent FGFR1 inhibitor with IC50 of ˜25 nM and also inhibits VEGFR2 with IC50 of 100-200 nM in cell-free assays, ˜1000-fold selective for FGFR1 than PDGFR and c-Src. The concentration of the PD173074 is usually in the concentration of about 100 nM to about 1000 nM, such as about 400 nM to about 600 nM.

In some embodiments, the mesodermal differentiation media comprises Hedgehog signaling agonist. Purmorphamine is the first small-molecule agonist developed for the protein Smoothened. Purmorphamine activates the Hedgehog (Hh) signaling pathway, resulting in the up- and downregulation of its downstream target genes, including Gli1 and Patched. The concentration of the Purmorphamine is usually in the concentration of about 0.5 μM to about 6 μM, such as about 1 μM to about 3 μM.

The concentration of serum, if present, is usually in the range of about 0.1 to about 2% v/v, such as about 0.1 to about 0.5%, about 0.2 to about 1.5% v/v, about 0.2 to about 1% v/v, about 0.5 to 1% v/v or about 0.5 to about 1.5% v/v. Serum may, for example, if present, in the mesodermal differentiation medium may be at a concentration of about 0.2% v/v, about 0.5% v/v or about 1% v/v. In one aspect, the differentiation medium omits serum and instead comprises a suitable serum replacement.

The culture medium forming the basis for the mesodermal differentiation medium may be any culture medium suitable for culturing PS cells and is not particularly limited. For example, base media such as Stem Pro-34 media, RPMI 1640 or advanced medium, Dulbecco's Modified Eagle Medium (DMEM), Iscove's Modified Dulbecco's Media (IMDM), F-12 Medium (also known as Ham's F-12), or MEM may be used. Thus, the differentiation medium may be StemPro-34 media or advanced medium comprising or supplemented with the above-mentioned components. In some embodiments, the base media may be a blend of two or more suitable culture medias, for example, the base media may be a blend of IMDM and F-12. In some embodiments, the differentiation medium may be DMEM or a blend comprising DMEM comprising or supplemented with the above-mentioned components. The differentiation medium may thus also be MEM medium or a blend comprising MEM comprising or supplemented with the above-mentioned components. In some embodiments, the differentiation medium may be IMDM or a blend comprising IMDM comprising or supplemented with the above-mentioned components. In some embodiments, the differentiation medium may be F-12 or a blend comprising F-12 comprising or supplemented with the above-mentioned components.

In some embodiments, the mesodermal differentiation medium comprises, consists essentially of, or consists of, a base medium supplemented with a defined lipid concentrate, insulin, selenous acid, monothioglycerol, penicillin/streptomycin, transferrin, Activin, CHIR99021, and FGF2. In other embodiments, the mesodermal differentiation medium comprises, consists essentially of, or consists of, a base medium supplemented with a defined lipid concentrate, insulin, selenous acid, monothioglycerol, penicillin/streptomycin, transferrin, SB505124, CHIR99021, FGF2 and dorsomorphin. In still other embodiments, the mesodermal differentiation medium comprises, consists essentially of, or consists of, a base medium supplemented with a defined lipid concentrate, insulin, selenous acid, monothioglycerol, penicillin/streptomycin, transferrin, SB505124, WNT-C59, dorsomorphin, and PD173074. In still other embodiments, the mesodermal differentiation medium comprises, consists essentially of, or consists of, a base medium supplemented with a defined lipid concentrate, insulin, selenous acid, monothioglycerol, penicillin/streptomycin, transferrin, purmorphamine and WNT-C59. In still other embodiments, the mesodermal differentiation medium comprises, consists essentially of, or consists of, a base medium supplemented with a defined lipid concentrate, insulin, selenous acid, monothioglycerol, penicillin/streptomycin, transferrin, and BMP4.

In another embodiment, the mesodermal differentiation medium comprises, consists essentially of, or consists of, a 1:1 IMDM & F12 supplemented with about 1% defined lipid concentrate, about 1% insulin/transferrin/selenous acid, monothioglycerol, penicillin/streptomycin, about 30 ng/ml Activin A, about 4 μM CHIR99021, and about 20 mg/ml FGF2. In other embodiments, the mesodermal differentiation medium comprises, consists essentially of, or consists of, 1:1 IMDM & F12 supplemented with about 1% defined lipid concentrate, about 1% insulin/transferrin/selenous acid, monothioglycerol, penicillin/streptomycin, about 2 μM SB505124, about 3 μM CHIR99021, about 20 ng/ml FGF2, and about 4 μM dorsomorphin. In still other embodiments, the mesodermal differentiation medium comprises, consists essentially of, or consists of, 1:1 IMDM & F12 supplemented with about 1% defined lipid concentrate, about 1% insulin/transferrin/selenous acid, monothioglycerol, penicillin/streptomycin, about 2 μM SB505124, about 1 μM WNT-WNT-C59, about 4 μM dorsomorphin, and about 500 nM PD173074. In still other embodiments, the mesodermal differentiation medium comprises, consists essentially of, or consists of, 1:1 IMDM & F12 supplemented with about 1% defined lipid concentrate, about 1% insulin/transferrin/selenous acid, monothioglycerol, penicillin/streptomycin, about 2 μM purmorphamine, and 1 μM WNT-C59. In still other embodiments, the mesodermal differentiation medium comprises, consists essentially of, or consists of, 1:1 IMDM & F12 supplemented with about 1% defined lipid concentrate, about 1% insulin/transferrin/selenous acid, monothioglycerol, penicillin/streptomycin, and about 20 ng/ml BMP4.

The PS cells are normally cultured for up to about 24 hours in suitable differentiation medium in order to obtain anterior primitive streak cells. For example, from about days 0-1 of differentiation, PS cells can be exposed to an Activin, a FGF, and a GSK3β inhibitor. On about days 1-2 of differentiation primitive streak cells are normally cultured for up to about 24 hours in suitable differentiation medium in order to obtain paraxial mesoderm cells. For example, from about days 1-2 of differentiation, primitive streak cells can be exposed to fresh media, with an inhibitor of the activin receptor-like kinase receptors, an ATP-competitive inhibitor of AMPK (AMP-activated protein kinase) and/or a selective inhibitor of bone morphogenetic protein (BMP) signaling, a FGF, and a GSK3β inhibitor. On about days 2-3 of differentiation paraxial mesoderm cells are normally cultured for up to about 24 hours in suitable differentiation medium in order to obtain early somite cells. For example, from about days 2-3 of differentiation, paraxial mesoderm cells can be exposed to fresh media, with an inhibitor of the activin receptor-like kinase receptors, an ATP-competitive inhibitor of AMPK (AMP-activated protein kinase) and/or a selective inhibitor of bone morphogenetic protein (BMP) signaling, and a WNT inhibitor. On about days 3-6 of differentiation early somite cells are normally cultured for up to about 3 days in suitable differentiation medium in order to obtain sclerotome cells. For example, from about days 3-6 of differentiation, early somite cells can be exposed to fresh media daily, with an Hh activator, and a WNT inhibitor. On about days 6-12 of differentiation sclerotome cells are normally cultured for up to about 6 days in suitable differentiation medium in order to obtain chondroprogenitor cells. For example, from about days 6-12 of differentiation, sclerotome cells can be exposed to fresh media daily, with a BMP. In some embodiments, the cells are cultured in a cell culture vessel coated with at least one extracellular matrix protein (e.g., vitronectin or Matrigel) during contact with the differentiation medium. The cells may be dissociated and collected in suspension (e.g., through contact with TrypLE, 0.05 mM EDTA), if needed.

### (c) Chondrogenesis

Following the mesoderm differentiation step, the obtained mesoderm cells can be further cultured in a chondrogenic differentiation medium to obtain chondrocyte and/or chondrocyte-like cells. In general, in order to obtain chondrocyte and/or chondrocyte-like cells, mesoderm cells, for example, chondroprogenitor cells as described above, are further cultured in chondrogenic differentiation medium. The mesoderm cells (e.g., chondroprogenitor cells) are prepared into a 3D culture. For example, cells are dissociated at the chondroprogenitor stage and are resuspended in chondrogenic medium. The cells can then be centrifuged under conditions which do not disrupt the cell integrity to form a pellet. The chondrogenic pellets can then be cultured under suitable conditions and timepoints (e.g., from about 1 to about 56 days) required for chondrocyte and/or chondrocyte-like cell production.

Generally, in order to obtain chondrocyte and/or chondrocyte-like cells, mesoderm cells such as chondroprogenitor cells can be cultured in a chondrogenic differentiation medium comprising non-essential amino acids, insulin, selenous acid, L-proline, transferrin, dexamethasone, L-ascorbic acid 2-phasphate, and a reducing agent such as β-mercaptoethanol. The chondrogenic differentiation medium may also comprise one or more growth factors, such as a transforming growth factor β (e.g., TGF-β1, TGF-β2 and TGF-β3), BMPs (e.g., BMP2, BMP4, BMP6, BMP7), one or more inhibitors of WNT signaling (e.g., sFRP, Dkk, WIF, Wise/SOST, Cerberus, IGFBP, Shisa, Waif1, APCDD1, Tiki1, and WNT-C59), and optionally an inhibitor of the microphthalmia-associated transcription factor (a.k.a, Melanocyte Inducing Transcription Factor; MITF) pathway (e.g., ML329). The concentration of the one or more growth factors may vary depending on the particular compound used. The concentration of TGF-β3, for example, is usually in the range of about 1 to about 20 ng/ml, such as about 5 to about 15 ng/ml. The concentration of BMP4, for example, is usually in the range of about 1 to about 40 ng/ml, such as about 5 to about 20 ng/ml. bFGF may, for example, be present in the differentiation medium at a concentration of about 10 ng/ml. The concentration of one or more WNT signaling inhibitors may vary depending on the particular compound used. For example, WNT-C59 is usually in the range of about 0.5 μM to about 4 μM, such as about 2 μM. The concentration of the one or more inhibitor of the MITF pathway may vary depending on the particular compound used. For example, ML329 is usually in the range of about 0.5 μM to about 4 μM, such as about 2 μM.

In some embodiments, the chondrogenic medium comprises, consists essentially of, or consists of, a base medium supplemented with a transforming growth factor beta, and a WNT signaling inhibitor. In another embodiment, the chondrogenic medium comprises, consists essentially of, or consists of a base medium supplemented with a transforming growth factor beta, a WNT signaling inhibitor and inhibitor of the MITF pathway. In another aspect, the chondrogenic medium consists essentially of, or consists of, a base medium supplemented with about 10 ng/ml TGFβ3 and 1 μM WNT-C59. In another aspect, the chondrogenic medium consists essentially of, or consists of, a base medium supplemented with about 10 ng/ml TGFβ3, 1 μM WNT-C59, and 20 ng/ml BMP-4. In another aspect, the chondrogenic medium consists essentially of, or consists of, a base medium supplemented with a base medium supplemented with about 10 ng/ml TGFβ3, 1 μM WNT-C59, and 1 μM ML329.

The culture medium forming the basis for the specification medium may be any culture medium suitable for culturing mesodermal cells and is not particularly limited. For example, the culture medium forming the basis for the specification medium may be any culture medium suitable for culturing ME cells and is not particularly limited. For example, base media such as Stem Pro-34 media, RPMI 1640 or advanced medium, Dulbecco's Modified Eagle Medium (DMEM), Iscove's Modified Dulbecco's Media (IMDM) F-12 Medium (also known as Ham's F-12), or MEM may be used. Thus, the differentiation medium may be StemPro-34 media or advanced medium comprising or supplemented with the above-mentioned components. In some embodiments, the base media may be a blend of two or more suitable culture medias, for example, the base media may be a blend of IMDM and F-12. In some embodiments, the differentiation medium may be DMEM or a blend comprising DMEM comprising or supplemented with the above-mentioned components. The differentiation medium may thus also be MEM medium or a blend comprising MEM comprising or supplemented with the above-mentioned components. In some embodiments, the differentiation medium may be IMDM or a blend comprising IMDM comprising or supplemented with the above-mentioned components. In some embodiments, the differentiation medium may be F-12 or a blend comprising F-12 comprising or supplemented with the above-mentioned components.

Chondrogenesis can be measure as described in the below examples, using techniques such as fluorescence-activated cell sorting (FACS).

### (d) Genetic Modification of Pluripotent Stem Cells or Chondroprogenitor Cells

In some embodiments, the pluripotent stem cells used in the in vitro culturing system disclosed herein or the chondrocyte and/or chondrocyte-like cells produced by the same may be genetically modified such that a gene of interest is modulated. Accordingly, the present disclosure also provides methods of preparing such genetically modified pluripotent stem cells or chondrocyte and/or chondrocyte-like cells. In some embodiments, the gene of interest is disrupted. As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation, or an intron region. Alternatively, the one or more mutations may be located in a coding region (e.g., in an exon). In some instances, the disrupted gene does not express or express a substantially reduced level of the encoded protein. In other instances, the disrupted gene expresses the encoded protein in a mutated form, which is either not functional or has substantially reduced activity. In some embodiments, a disrupted gene does not express (e.g., encode) a functional protein.

Techniques such as CRISPR (particularly using Cas9 and guide RNA), editing with zinc finger nucleases (ZFNs), and transcription activator-like effector nucleases (TALENs) may be used to produce the genetically engineered pluripotent stem cells.

‘Genetic modification’, ‘genome editing’, or ‘genomic editing’, or ‘genetic editing’, as used interchangeably herein, is a type of genetic engineering in which DNA is inserted, deleted, and/or replaced in the genome of a targeted cell. Targeted genome modification (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. In another aspect, an endogenous gene may be modified by introducing a change in an endogenous gene codon, wherein the modification introduces an amino acid change in the gene product or introduction of a stop codon. Therefore, targeted modification may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and reliable gene response control.

Targeted modification can be achieved either through a nuclease-independent approach or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted modification could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

In some embodiments, non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

In an exemplary embodiment, the CRISPR/Cas9 gene editing technology is used for producing the genetically engineered pluripotent stem cells. Typically, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. Any known CRISPR/Cas9 methods can be used in the methods disclosed herein. See also Examples below.

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases, homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap, or hybrid selection. ZFNs are described in greater detail in U.S. Pat. Nos. 7,888,121 and 7,972,854. The most recognized example of a ZFN is a fusion of the Fokl nuclease with a zinc finger DNA binding domain.

A TALEN is a targeted nuclease comprising a nuclease fused to a TAL effector DNA binding domain. A “transcription activator-like effector DNA binding domain,” “TAL effector DNA binding domain,” or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the Fokl nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Any of the gene editing nucleases disclosed herein may be delivered using a vector system, including, but not limited to, plasmid vectors, DNA minicircles, retroviral vectors, lentiviral vectors, adenovirus vectors, poxvirus vectors, herpesvirus vectors, and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

### II. Methods of Use

Any of the chondrocyte and/or chondrocyte-like cell produced by the methods of various aspects described herein (e.g., the methods of Section I) can be used in different applications where chondrocyte and/or chondrocyte-like cell are required. Such uses of chondrocyte and/or chondrocyte-like cell are also within the scope of the present disclosure.

In some embodiments, the chondrocyte and/or chondrocyte-like cell are obtained from cells derived from a subject to whom the chondrocyte and/or chondrocyte-like cell are to be administered. In such embodiments, the embryonic stem cells can be derived from ESC, iPSC, or reprogrammed non-pluripotent cells derived from the subject to whom the chondrocyte and/or chondrocyte-like cell derived therefrom are to be administered. In a specific embodiment, adult cells can be obtained from a subject, such cells can be reprogrammed to iPSC and then produced into chondrocyte and/or chondrocyte-like cell of the disclosure. In specific embodiments, chondrocyte and/or chondrocyte-like cell are derived from cells of a deceased patient. In specific embodiments, chondrocyte and/or chondrocyte-like cell are derived from cells of a patient with a disease, disorder, or injury, and such chondrocyte and/or chondrocyte-like cells are produced for administration to the subject.

The thus-obtained chondrocyte and/or chondrocyte-like cells, when applied to cartilage tissue in vivo, can form a new cartilage tissue of a three-dimensional structure using the cartilage tissue as a scaffold. When cultured in vitro in the presence of a scaffolding material, the chondrocyte and/or chondrocyte-like cells can form a cartilage tissue of a three-dimensional structure.

As described thus far, the chondrocyte and/or chondrocyte-like cells obtained in the present disclosure have a proliferative ability and can regenerate cartilage tissue in an organism. The chondrocyte-like cells are thus effective for the treatment of cartilage disease such as arthritis (e.g., osteoarthritis), osteochondrosis deformans, chondrodystrophy arthritis (e.g., rheumatoid arthritis), trauma, and osteonecrosis, and can be used as a cell preparation (pharmaceutical composition) for cartilage tissue regeneration. The chondrocyte and/or chondrocyte-like cells may be applied to a cartilage disease site either alone or with a scaffolding material. When applied to a cartilage disease site with a scaffolding material, the chondrocyte-like cells may be applied to the cartilage disease site separately from the scaffolding material. However, it is desirable that the chondrocyte and/or chondrocyte-like cells and the scaffolding material be applied to the cartilage disease site at the same time in the form of a cell preparation, as will be described later.

When the chondrocyte and/or chondrocyte-like cells are prepared as a cell preparation for cartilage tissue regeneration, a pharmaceutically acceptable carrier for dilution may be contained with the chondrocyte and/or chondrocyte-like cells, as required. Examples of pharmaceutically acceptable carriers for dilution include physiological saline and buffers. Further, the cell preparation may also contain pharmacologically active components, growth factors, small molecule inhibitors, and nutrient source components for the chondrocyte and/or chondrocyte-like cells, as required.

Desirably, the cell preparation contains a scaffolding material for the chondrocyte and/or chondrocyte-like cells. When the cell preparation contains a scaffolding material, it is desirable that the chondrocyte-like cells be contained by being supported on the scaffolding material. The use of scaffolding material improves the graft rate of the chondrocyte and/or chondrocyte-like cells at the diseased site of cartilage tissue and further promotes cartilage tissue regeneration.

The scaffolding material is not particularly limited, as long as it is pharmaceutically acceptable. The scaffolding material is appropriately selected according to the target site of cartilage tissue. For example, gelatinous or porous, biodegradable or bioresorbable materials can be used. Preferred examples of scaffolding material include collagen, hydroxyapatite, α-TOP (tricalcium phosphate), β-TOP (tricalcium phosphate), polylactic acid, polyglycolic acid, and complexes of these. The scaffolding materials may be used either alone or in combinations of two or more. Of these scaffolding materials, collagen is preferable from the standpoint of efficient cartilage tissue regeneration. When collagen is used as scaffolding material, the collagen is desirably prepared into a gel form of a three-dimensional structure.

The shape of the scaffolding material is not particularly limited, and is appropriately designed according to the shape of the damaged site of the cartilage tissue targeted by the cell preparation.

The chondrocyte and/or chondrocyte-like cells can be supported on the scaffolding material by, for example, inoculating or mixing the chondrocyte and/or chondrocyte-like cells with the scaffolding material, followed by culturing.

When the chondrocyte and/or chondrocyte-like cells in the cell preparation are used by being supported on the scaffolding material or used to construct a cartilage tissue of a three-dimensional structure, the proportion of the chondrocyte and/or chondrocyte-like cells with respect to the scaffolding material may be appropriately set according to such factors as the site of the targeted cartilage tissue and the type of scaffolding material. As an example, the chondrocyte-like cells are used in a proportion of 1×10⁶ to 1×10⁸ cells per 1 cm³ of the scaffolding material.

The method used to apply the cell preparation to the diseased site of cartilage tissue is appropriately set according to such factors as the type of cell preparation and the site of the targeted cartilage tissue. For example, the cell preparation may be directly injected through an incision at the diseased site of the treated cartilage tissue or the cell preparation may be injected to the diseased site of the treated cartilage tissue using an arthroscope.

The dose of the cell preparation applied to the diseased site of cartilage tissue may be appropriately set to an amount effective for cartilage tissue regeneration, based on such factors as the type of cell preparation, the site of cartilage tissue, the extent of symptoms, and the age and sex of a patient.

Further, the chondrocyte and/or chondrocyte-like cells may be used to construct a cartilage tissue of a three-dimensional structure in vitro, and this construct may be used as a cartilage tissue implant for the treatment of cartilage disease that involves cartilage defects such as in osteochondrosis deformans.

The chondrocyte and/or chondrocyte-like cells can be used to construct a cartilage tissue of a three-dimensional structure by, for example, inoculating the chondrocyte-like cells in scaffolding material and culturing the cells in a medium capable of growing a chondrocyte-like cell until a cartilage tissue of a three-dimensional structure is constructed. More specifically, about 1×10⁶ to 1×10⁸ chondrocyte and/or chondrocyte-like cells may be inoculated per 1 cm³ of scaffolding material and cultured in normoxic (21% O₂) or hypoxic oxygen conditions (1-5% O₂) under 5% CO₂ conditions at 37° C. for about 1 to 4 weeks. The same scaffolding material used for the cell preparation can be used to construct a cartilage tissue of a three-dimensional structure. The shape of the scaffolding material may be appropriately set according to the shape of the implant of interest. The medium used to construct a cartilage tissue of a three-dimensional structure is not particularly limited, as long as it can grow the chondrocyte-like cells. For example, DMEM medium containing about 1 to 25 volume % FBS may be used. From the standpoint of clinical application, use of serum-free media of defined compositions (defined serum-free media) is desirable.

The thus-prepared cartilage tissue of a three-dimensional structure prepared as above is used as a cartilage tissue implant, either in the state containing the scaffolding material or after removing the scaffolding material.

The method used to apply the implant to the diseased site of the cartilage tissue is appropriately set according to such factors as the shape of the implant and the site of the targeted cartilage tissue. For example, the implant may be directly incorporated through an incision at the diseased site of the treated cartilage tissue.

The chondrocyte and/or chondrocyte-like cells also can form a cartilage tissue when administered to a site of an organism other than the cartilage tissue. Thus, the chondrocyte and/or chondrocyte-like cells may be administered into the body of a mammal, and the cartilage tissue formed by the chondrocyte-like cells in the body of the mammal may be removed to obtain a cartilage tissue implant.

The mammals used for the production of such cartilage tissue implants may be humans or non-human mammals such as mice, rats, hamsters, rabbits, cats, dogs, sheep, pigs, cows, goats, horses, and monkeys. Further, in the production of the cartilage tissue implant, the administration site of the chondrocyte and/or chondrocyte-like cells is not particularly limited. However, considering the ease of the removal of the newly formed cartilage tissue, the administration site is preferably under the skin, particularly under the skin of the back. Further, in the production of the cartilage tissue implant, the chondrocyte-like cells may be administered together with a scaffolding material or alone without a scaffold. The chondrocyte and/or chondrocyte-like cells can form a cartilage tissue of a sufficient size in an organism without the administration of a scaffold.

In the production of the cartilage tissue implant, the dose of the chondrocyte-like cells for mammals is not particularly limited, and may be generally about 10⁴ to 10⁸ cells, preferably about 10⁵ to 10⁷ cells. Formation of a cartilage tissue is recognized after 14 to 35 days, preferably after 21 to 28 days from the administration of the chondrocyte and/or chondrocyte-like cells to mammals.

The cartilage tissue implant may be produced in the body of a cartilage disease patient, and the cartilage tissue so produced may be transplanted into the cartilage disease site of the patient. Specifically, the chondrocyte and/or chondrocyte-like cells may be administered to a site of a cartilage disease patient other than the cartilage tissue, and a new cartilage tissue formed by the chondrocyte and/or chondrocyte-like cells in the body of the patient may be removed and administered to the cartilage disease site of the patient for the graft treatment of cartilage disease.

Further, a non-human mammal including a cartilage tissue formed by the chondrocyte and/or chondrocyte-like cells administered to the organism may be used as a tool for evaluating the efficacy of a tested substance for the cartilage tissue. Specifically, a non-human mammal that includes a cartilage tissue formed by the chondrocyte and/or chondrocyte-like cells may be administered with a tested substance to determine and evaluate the efficacy of the tested substance for the cartilage tissue. As used herein, the “tested substance” refers to a substance to be evaluated for its efficacy for the cartilage tissue. Specific examples include a candidate substance of a therapeutic drug for cartilage disease.

Further, the chondrocyte and/or chondrocyte-like cells can be used as a tool for elucidating the pathology of various cartilage diseases. The chondrocyte-like cells induced from human somatic cells are useful as a tool for the discovery and development of drugs for cartilage diseases.

Once generated the chondrocyte and/or chondrocyte-like cell be cryopreserved in accordance with the methods described below or known in the art.

In one embodiment, a chondrocyte and/or chondrocyte-like cell population can be divided and frozen in one or more bags (or units). In another embodiment, two or more chondrocyte and/or chondrocyte-like cells populations can be pooled, divided into separate aliquots, and each aliquot is frozen. In a preferred embodiment, a maximum of approximately 4 billion nucleated cells is frozen in a single bag. In a preferred embodiment, the chondrocyte and/or chondrocyte-like cells are fresh, i.e., they have not been previously frozen prior to expansion or cryopreservation. The terms “frozen/freezing” and “cryopreserved/cryopreserving” are used interchangeably in the present application. Cryopreservation can be by any method in known in the art that freezes cells in viable form. The freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. For a discussion, see Mazur, P., 1977, Cryobiology 14:251-272.

These injurious effects can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann, N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962, Fed. Proc. 21:157), D-sorbitol, i-inositol, D-lactose, choline chloride (Bender et al., 1960, J. Appl. Physiol. 15:520), amino acids (Phan The Tran and Bender, 1960, Exp. Cell Res. 20:651), methanol, acetamide, glycerol monoacetate (Lovelock, 1954, Biochem. J. 56:265), and inorganic salts (Phan The Tran and Bender, 1960, Proc. Soc. Exp. Biol. Med. 104:388; Phan The Tran and Bender, 1961, in Radiobiology, Proceedings of the Third Australian Conference on Radiobiology, Ilbery ed., Butterworth, London, p. 59). In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After addition of DMSO, cells should be kept at 0° C. until freezing, since DMSO concentrations of about 1% are toxic at temperatures above 4° C.

A controlled slow cooling rate can be critical. Different cryoprotective agents (Rapatz et al., 1968, Cryobiology 5(1):18-25) and different cell types have different optimal cooling rates (see e.g., Rowe and Rinfret, 1962, Blood 20:636; Rowe, 1966, Cryobiology 3(1):12-18; Lewis, et al., 1967, Transfusion 7(1):17-32; and Mazur, 1970, Science 168:939-949 for effects of cooling velocity on survival of marrow-stem cells and on their transplantation potential). The heat of fusion phase where water turns to ice should be minimal. The cooling procedure can be carried out by use of e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1° to 3° C./minute from 0° C. to −80° C. In a preferred embodiment, this cooling rate can be used for CB cells. The container holding the cells must be stable at cryogenic temperatures and allow for rapid heat transfer for effective control of both freezing and thawing. Sealed plastic vials (e.g., Nunc, Wheaton cryules) or glass ampules can be used for multiple small amounts (1-2 ml), while larger volumes (100-200 ml) can be frozen in polyolefin bags (e.g., Delmed) held between metal plates for better heat transfer during cooling. Bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred embodiment, DMSO-treated cells are pre-cooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1° to 3° C./minute. After at least two hours, the specimens have reached a temperature of −80° C. and can be placed directly into liquid nitrogen (−196° C.) for permanent storage.

After thorough freezing, the chondrocyte and/or chondrocyte-like cells can be rapidly transferred to a long-term cryogenic storage vessel. In a preferred embodiment, samples can be cryogenically stored in liquid nitrogen (−196° C.) or its vapor (−165° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

Suitable racking systems are commercially available and can be used for cataloguing, storage, and retrieval of individual specimens.

Following cryopreservation, frozen isolated hematopoietic progenitor cells can be thawed in accordance with the methods described below or known in the art.

Frozen cells are preferably thawed quickly (e.g., in a water bath maintained at 37°−41° C.) and chilled immediately upon thawing. In a specific embodiment, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Single Cell Transcriptomic Analysis of Human Pluripotent Stem Cell Chondrogenesis

The therapeutic application of human induced pluripotent stem cells (hiPSCs) for cartilage regeneration is largely hindered by the low yield of chondrocytes accompanied by unpredictable and heterogeneous off-target differentiation of cells during chondrogenesis. The present example combines bulk RNA sequencing, single cell RNA sequencing, and bioinformatic analyses, including weighted gene co-expression analysis (WGCNA), to investigate the gene regulatory networks regulating hiPSC differentiation under chondrogenic conditions. specific WNTs and MITF as hub genes are identified as governing the generation of off-target differentiation into neural cells and melanocytes during hiPSC chondrogenesis. With heterocellular signaling models, the present example further shows that WNT signaling produced by off-target cells is responsible for inducing chondrocyte hypertrophy. By targeting WNTs and MITF, these cell lineages are eliminated, significantly enhancing the yield and homogeneity of hiPSC-derived chondrocytes. Collectively, the present example provides the trajectories and molecular mechanisms governing cell fate decision in hiPSC chondrogenesis, as well as dynamic transcriptome profiles orchestrating chondrocyte proliferation and differentiation.

### Methods

hiPSC lines and culture. Three distinct hiPSC lines were used in the current study: STAN, ATCC, and BJFF. STAN line was purchased from WiCell (#STAN061i-164-1), ATCC line was acquired from ATCC (#ATCCACS-1019), and BJFF was obtained from the Genome Engineering and iPSC Core at Washington University in Saint Louis. All three lines were reprogrammed by Sendai virus from human foreskin fibroblasts and confirmed to be karyotypically normal and mycoplasma free. STAN and BJFF hiPSCs were maintained on vitronectin coated 6-well plates (Thermo Fisher Scientific, #A31804) in Essential 8 Flex medium (Thermo Fisher Scientific, #A2858501). ATCC hiPSCs were cultured on CellMatrix Basement Membrane Gel coated 6-well plates (ATCC, #ACS3035) in Pluripotent Stem Cell SFM XF/FF medium (ATCC, #ACS3002). Cells were fed daily and passaged with ReLeSR (STEMCELL Technologies, #05872). All hiPSC lines were maintained below passage 30.

hMSCs and culture. Discarded and deidentified waste tissue from the iliac crests of adult bone marrow transplant donors was collected in accordance with the institutional review board of Washington University in Saint Louis. Human bone marrow-derived MSCs (hMSCs) were isolated by their physical adherence to plastic culture vessels. Cells were expanded and maintained in an expansion medium consisting of DMEM-low glucose (Thermo Fisher Scientific, #11885092), 1 penicillin/streptomycin (P/S, Thermo Fisher Scientific, #15140-122), 10% lot-selected FBS (Atlanta Biologicals, #S11550), and 1 ng ml⁻¹ basic fibroblast growth factor (FGF) (R&D Systems, #233-FB). Three individual donors were used as biologic replicates in subsequent experiments.

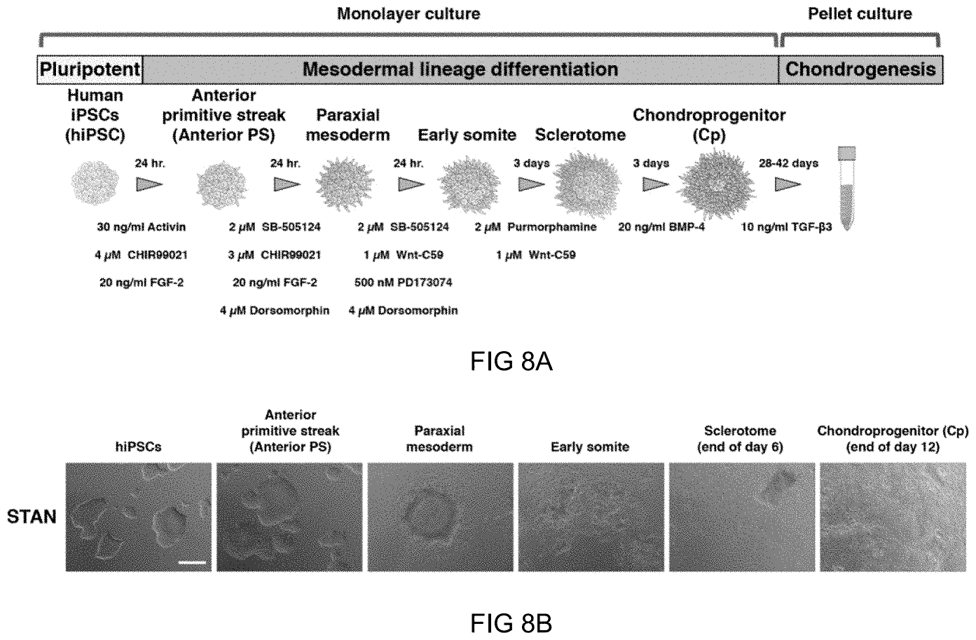

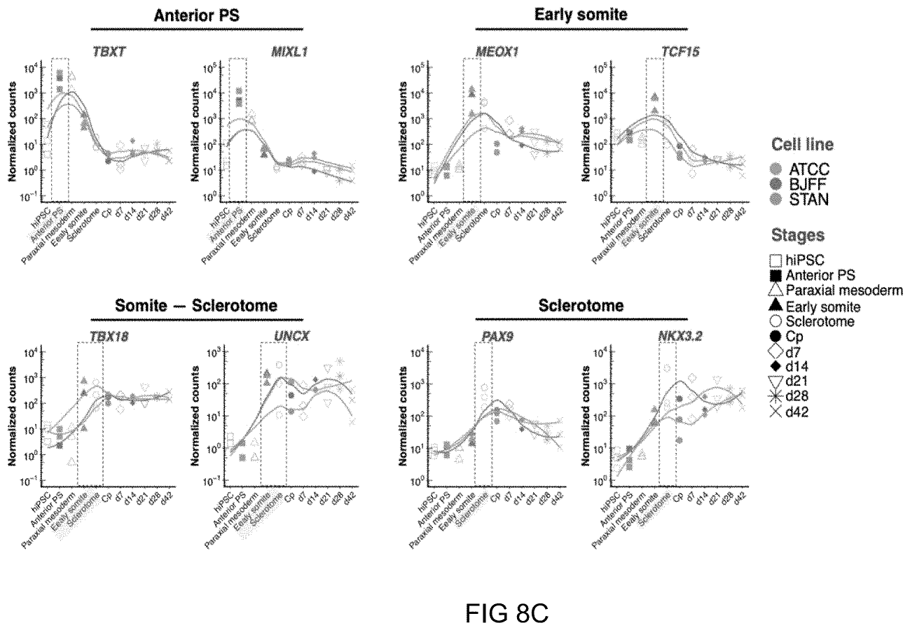

Mesodermal differentiation. hiPSCs were induced into mesodermal differentiation in monolayer at 40% confluency. Each day, cells were rinsed with a wash medium consisting of 50% IMDM GlutaMAX (IMDM, Fisher Scientific, #31980097) and 50% Ham's F12 Nutrient Mix (F12, Fisher Scientific, #31765092) to remove the previous medium. hiPSCs were then fed daily to sequentially drive mesodermal differentiation similar to those identified in embryonic development with various sets of growth factors and small molecules supplemented in mesodermal differentiation medium consisting of equal parts of IMDM and F12 with 1% chemically defined lipid concentrate (Gibco), 1% insulin/human transferrin/selenous acid (ITS+, Corning, #354352), 1% P/S (Thermo Fisher Scientific, #15140-122), and 450 μM 1-thioglycerol (Sigma-Aldrich, #M6145). Cells were induced to the anterior primitive streak with 30 ng ml⁻¹ of Activin A (R&D Systems, #338-AC), 4 μM CHIR99021 (Stemgent, #04-0004), and 20 ng ml⁻¹ human FGF-2 (R&D Systems, #233-FB-025/CF) for 24 h. On the second day, cells were driven to paraxial mesoderm with 2 μM SB-505124 (SB5; Tocris, #3263), 3 μM CHIR99021, 20 ng ml⁻¹ human FGF-2, and 4 μM dorsomorphin (DM; Stemgent, #04-0024). Then, cells were treated with 2 μM SB5, 4 μM DM, 1 μM WNT-C59 (Cellagent Technology, #C7641-2s), and 500 nM PD173074 (Tocris, #3044) to become early somite on the third day. For the fourth through sixth days, cells were driven to the sclerotome with daily feedings of 2 μM purmorphamine (Stemgent, #04-0009) and 1 μM WNT-C59. Finally, for six days, cells were driven to the Cp stage with 20 ng ml⁻¹ of human bone morphogenetic protein 4 (BMP4; R&D Systems, #314-BP-010/CF) daily (FIG. 8A).

At each stage, cells were dissociated using TrypLE (Gibco, #12604013) at 37° C. for 3 min followed by adding an equal part of neutralizing medium consisting of DMEM/F-12, GlutaMAXTM (DMEM/F12; Thermo Fisher Scientific, #10565042) with 10% FBS (Atlanta Biologicals) and 1% P/S. The dissociated cells were either used for bulk RNA-seq, scRNA-seq, chondrogenic differentiation, or fluorescence-activated cell sorting (FACS) as appropriate.

Chondrogenic differentiation. Cells dissociated at the Cp stage were resuspended at 5×10⁵ cells per mL in chondrogenic medium consisting of DMEM/F-12, 1% FBS, 1% ITS+, 55 μM β-mertcaptoethanol, 100 nM dexamethasone (DEX; Sigma-Aldrich, #D4902), 1% NEAA (Gibco, #11140050), 1% P/S, 10 ng ml⁻¹ human transforming growth factor-beta 3 (TGF-β3; R&D Systems, #243-B3-010), 50 μg ml⁻¹ L-ascorbic acid 2-phosphate (ascorbate; Sigma-Aldrich, #A8960), and 40 μg ml⁻¹ L-Proline (proline; Sigma-Aldrich, #P5607). Cells were then centrifuged for 5 min at 300×g to form a pellet. Chondrogenic pellets were cultured at 37° C. for up to the timepoints required for various experiments.

On the day of collection for bulk RNA-seq experiments, 3-4 pellets per experimental group were pooled together and washed once with phosphate-buffered saline (PBS), snap-frozen in 300 μl of Buffer RL (Norgen Biotek), and stored at −80° C. until processing for RNA extraction. At harvesting time points for scRNA-seq experiments, 6-8 pellets per experimental group were pooled and digested with 0.04% Type II collagenase solution in DMEM/F12 for 1 h. Cells were washed once with PBS, resuspended in standard freezing medium, and stored in liquid nitrogen until needed.

WNT-C59 and ML329 treatment for WNT and MITF inhibition. For WNT-C59 treatment for WNT inhibition during chondrogenesis, pellets were treated with either 10 ng ml⁻¹ TGF-β3 (control group) or a combination of 10 ng ml⁻¹ TGF-β3 and 1 μM WNT-C59 in a chondrogenic medium from d0 to d42 as appropriate. For WNT-C59 and ML329 treatment (ML, Axon Medchem, HY-101464) for WNT and MITF inhibition during chondrogenesis, pellets were treated with either 10 ng ml⁻¹ TGF-β3 (control group), a combination of 10 ng ml⁻¹ TGF-β3 and 1 μM ML, a combination of 10 ng ml⁻¹ TGF-β3 and 1 μM WNT-C59, or a combination of 10 ng ml⁻¹ TGF-β3, 1 μM ML and 1 μM WNT-C59 in chondrogenic medium from d0 to d42 as appropriate.

WNT ligands treatment during chondrogenesis. For WNT ligands treatment during chondrogenesis, pellets were treated with either 10 ng ml⁻¹ TGF-β3 (control group) or a combination of 10 ng ml⁻¹ TGF-β3 and 100 ng ml⁻¹ individual WNT ligand (WNT2B, WNT3A, WNT4, WNT5B, or WNT7B, all from R&D system) in chondrogenic medium from d0 to d42 as appropriate. For WNT ligands treatment during the Cp stage, cells were supplemented with either 20 ng ml⁻¹ BMP4 (R&D Systems, #314-BP-010) alone (control group), a combination of 20 ng ml⁻¹ BMP4 and 1 μM WNT-059, or a combination of 20 ng ml⁻¹ BMP4 and 100 ng ml⁻¹ WNT3A (R&D Systems, #5036-WN-010) in mesodermal differentiation medium from d7 to d12.

Animal experiments. All animal procedures were approved by the Institutional Animal Care and Use Committee (IACUC) at Washington University in Saint Louis. Male NSG mice (NOD.Cg-Prkdcscid Il2rgtm1WjI/SzJ, #005557, Jackson laboratory) at age of 18-20 weeks old were used for human xenograft implantation in the dorsal region (subcutaneous) or in osteochondral defects in the knee joints of mice. Mice were housed under a 12 h light/12 h dark cycle with ambient temperature and humidity. NSG mice were anesthetized with 3% isoflurane in oxygen for all surgical procedures. For subcutaneous implantation, the skin was shaved and sterilized over the implantation site using standard sterile techniques. A mid-scapular incision was made, and a hemostat was inserted into the skin incision to create a pocket for implantation. A d14 hiPSC chondrogenic pellet was then inserted into the pockets. The incision of the skin was closed with 8-0 suture with taper point (PolysorbTM, Covidien, #L-2800). Tissue adhesive was applied to the skin wound area. For implantation in osteochondral defects in the knee, a 3 mm long medial parapatellar incision was made in the left hindlimb, and the knee joint was exposed via lateral dislocation of the patella. An osteochondral defect (1 mm in diameter and 1 mm in depth) in the trochlear groove of the femur was created by a 1 mm micro bone drill (Roboz, #RS-6300A). All debris was removed by sterile PBS washes. Mild hemorrhage from the fat pad was controlled by epinephrine 1:1000 (International Medication Systems, #491590) followed by sterile PBS wash. A d14 hiPSC chondrogenic pellet was implanted into the defect, and the patella was repositioned to its original anatomical location. Mice with osteochondral defects that did not receive pellet implantation were used as a control group. After implantation, the subcutaneous layer and skin were closed with 8-0 suture with a tapered point followed by the application of tissue adhesive to the skin wound area. After surgery, the mice were allowed to move freely within their cages. After 14 and 28 days post implantation, mice were sacrificed for pellet harvest for histological analysis.

RNA isolation, library preparation, and bulk RNA-seq. To determine transcriptome profiles over the course of differentiation, three hiPSCs lines (ATCC, BJFF, and STAN) as biological replicates at various differentiation stages (6 mesodermal and 5 chondrogenic stages per cell line; i.e., total 33 samples) were collected for bulk RNA-seq. Cell samples were thawed on ice, and pellet samples were homogenized with zirconia beads (BioSpec Products, #11079110zx) and a miniature bead beater. RNA was then isolated from all samples using the Total RNA Purification Kit according to the manufacture's protocol (Norgen Biotek, #37500). RNA was eluted in 20 μl of diethylpyrocarbonate-treated water. The quality and quantity of RNA from each sample was evaluated by RNA Analysis ScreenTape (Agilent, #5067-5576) on a bioanalyzer (Agilent 4200 Tapestation). Only samples with a RIN value larger than 0.8 were submitted to the Genome Technology Access Center (GTAC sequencing core) at Washington University in St. Louis for library preparation and bulk RNA-seq. Libraries were prepared using TruSeq Stranded Total RNA with Ribo-Zero Gold kit (IIlumina). Sequencing was performed on a HiSeq2500 instrument (Illumina) (1×50 bp reads) with a sequencing depth of 30 million reads per sample.

Preprocessing of bulk RNA-seq data. Reads were processed using an in-house pipeline and open-source R packages. Raw reads were first trimmed using Cutadapt to remove low-quality bases and reads. After trimming, processed reads were aligned to the human reference genome GRCh38 (version 90) by STAR50, and the number of aligned reads to each annotated genes or transcripts (GENCODE v21) was performed using featureCounts from the Subread package (v1.4.6).

DEGs and GO enrichment analysis and of bulk RAN-seq data. After quality control, un-normalized gene counts were read into the DESeq2 R package by DESeqDataSetFromMatrix function as instructed by the package tutorial. Genes that were expressed by less than ten cells were then removed. Next, DESeq was used and results functions which implement Wald test in DESeq2 to determine the DEGs between two consecutive differentiation stages. In this process, the estimation of size factors (i.e., controlling for differences in the sequencing depth of the samples), the estimation of dispersion values for each gene, and fitting a generalized linear model were performed. The gene counts were also averaged from three hiPSC lines. Top 20 DEGs between two consecutive stages were selected and visualized using ComplexHeatmap R package. To observe the temporal expression of a given gene for each hiPSC line, the count matrix was regularized-logarithm transformed via rlog function first, and plotCounts function in DESeq2 were used to visualize the expression pattern of the gene. Furthermore, regularized-logarithm transformed counts were also used for PCA, and PCA plots were visualized by ggplot function in ggplot2 R package.

We next performed GO enrichment analysis of the genes in mesodermal and chondrogenic stages using GAGE R package (Generally Applicable Gene-set/Pathway Analysis), whose algorism evaluates the coordinated up- or down-differential expression over gene sets defined by GO terms. Significantly upregulated GO terms with their associated p values in biological process, molecular function, and cellular component were plotted by GraphPad Prism (version 8.0; GraphPad Software). Furthermore, GAGE analysis also reveals that 134 out of 205 genes defined by GO term cartilage development (GO:0051216) were significantly increased during our differentiation process. Thus, a heatmap was generated to investigate the expression levels of these genes at various stages using ComplexHeatmap R package.

10× chromium platform scRNA-seq. Cells were thawed at 37° C. and resuspended in PBS with 0.04% bovine serum albumin at a concentration of 2000 cells per μl. Cell suspensions were submitted to the GTAC sequencing core at Washington University in St. Louis for library preparation and sequencing. In brief, 10,000 cells per sample were loaded on a Chromium Controller (10× Genomics) for single capture. Libraries were prepared using Single Cell 3′ Library & Gel Bead Kit v2 (#120237 10× Genomics) following the manufacture's instruction. A single cell emulsion (Gel Bead-In-EMulsions, GEMs) is created by making barcoded cDNA unique to each individual emulsion. A recovery agent was added to break GEM and cDNA was then amplified. A library is produced via end repair, dA-tailing, adapter ligation, post-ligation cleanup with SPRIselect, and sample index PCR. The quality and concentration of the amplified cDNA were evaluated by Bioanalyzer (Agilent 2100) on a High Sensitivity DNA chip (Agilent, #5065-4401). The only cDNA with an average library size of 260-620 bp were used for sequencing. Sequencing was performed by IIlumina HiSeq2500 with the following read length: 26 bp for Read1, 8 bp for i7 Index, and 98 bp for Read2. We generally acquired ˜180 million reads per library (sample). A species mixing experiment (mouse adipose stem cells and human iPSCs, 1:1 mixture) was also performed prior to running on the actual sample to ensure good quality of single-cell capture (i.e., cell doublet rate<5%).

Preprocessing of scRNA-seq data. Paired-end sequencing reads were processed by Cell Ranger (10× Genomics software, version 2.0.0). Reads were aligned to the GRCh38 (version 90) for genome annotation, demultiplexing, barcode filtering, and gene quantification. Cell Ranger also removes any barcode that has less than 10% of the 99th percentile of total unique molecular identifiers (UMI) counts per barcode as these barcodes are considered to be associated with empty droplets. After this quality control, gene barcode matrices for each sample were generated by counting the number of UMIs for a given gene (as a row) in the individual cell (as a column). For each sample, ˜1300-2500 cells were captured.

Unsupervised clustering analysis and annotation. To assess the difference in the composition of cell populations, global unsupervised clustering analysis was performed for our scRNA-seq datasets. First, gene barcode matrices were input into the Seurat R package (version 2.4). Then, the low-quality cells with less than 200 or more than 7000 detected genes or if their mitochondrial gene content was more than 5% were removed. Note that the cutoff criteria were adjusted in few cases due to the sequencing depth and the variations in mitochondrial gene content from datasets. Genes that were detected in less than three cells were filtered out. After filtering out low-quality cells or cell doublets, the gene expression was then natural log-transformed and normalized for scaling the sequencing depth to 10,000 molecules per cell. Next, to reduce the variance introduced by unwanted sources, variation in gene expression driven by cell cycle stages and mitochondrial gene expression were regressed out with vars.to.regress argument in function ScaleData in Seurat. Then the FindVariableGenes function in Seurat were used to identify highly variable genes across cells for downstream analysis. These steps resulted in (1) a total of 8547 cells with an average of 1882 highly variable genes from stages of hiPSCs, Sclerotome, and Cp stages, (2) a total of 10,648 cells with an average of 2061 highly variable genes from stages of TGF-β3-treated pellets (dl, d3, d7, d14, d28, and d42), and (3) total 7997 cells with average 1886 highly variable genes from TGF-β3+WNT-C59-treated pellets (d7, d14, d28, and d42) for downstream analysis. Dimensionality reduction on the data was then performed by computing the significant principal components on highly variable genes. We then performed unsupervised clustering by using the FindClusters function in Seurat with the resolution argument set to 0.6, and clusters were then visualized in a tSNE plot55.

DEGs among each cell cluster were determined using the FindAllMarkers function in Seurat. DEGs expressed in at least 25% of cells within the cluster and with a fold change of more than 0.25 in natural log scale were considered to be marker genes of the cluster. To determine the biological functions of the marker genes from a given cluster, we performed GO enrichment analysis by using The DAVID Gene Functional Classification Tool (http://david.abcc.ncifcrf.gov; version 6.8). By comparing these unique biological GO terms with existing RNA-seq datasets and the literature, we were able to annotate cell clusters. In addition, the top 10 enriched GO terms from the biological function category with associated p values were visualized GraphPad Prism (version 8.0; GraphPad Software).

Cell cycle analysis of scRNA-seq data. Cell cycle scoring function in Seurat was used to determine a cell cycle score on each cell according to its gene expression of G2/M phase (54 genes) and S phase (43 genes) markers. Based on this scoring system, fractions of each cell cluster with a given cell cycle score in total cell population were computed.

CCA for integrated analysis of multiple scRNA-seq datasets. To compare cell types and to identify their associated DEGs between distinct experimental conditions such as batch effect, WNT-C59 treatment, or differentiation stages (i.e., time points), we applied CCA, a computational strategy implemented in Seurat for integrated analysis of multiple datasets. First, the top 1000 highly variable genes from each dataset were selected. We then use the RunCCA function or RunMultiCCA function (if more than two datasets) to identify common sources of variation resulting from experimental conditions and to merge the multiple objects into a single dataset. We next determined the top principal components of the CCA by examining a saturation in the relationship between the number of principal components and the percentage of the variance explained using the MetageneBicorPlot function. By using selected top principal components, we aligned the CCA subspaces with the AlignSubspace function, which returns a new dimensional reduction matrix allowing for downstream clustering and DEG analyses. DEG analysis was performed on the cells from different datasets but grouped in the same cluster (i.e., conserved cell types between two conditions) after CCA alignment. The methods for cell clustering, identification of conserved cell types and DEGs, as well as annotation of cell clusters were similar to the ones mentioned previously. DEGs in each conserved cell type in response to differentiation stages or WNT-C59 treatment were visualized by ComplexHeatmap R package. In some cases, genes of interest such as WNTs and various lineage markers were also visualized using the FeatureHeatmap and DotPlot function in Seurat.

Pseudotemporal ordering and lineage trajectories. We used the Monocle2 R package to reconstruct differentiation trajectories by computing and ordering the sequence of gene expression changes of the cells collected from different time points in an unsupervised manner. First, scRNA-seq datasets from different timepoints underwent several quality control steps as mentioned previously. These multiple scRNA-seq datasets were then merged into one single object using the MergeSeurat function in Seurat. The merged matrix was then converted into a Monocle object using importCDS and newCellDataSet functions in Monocle2. We then identified a set of DEGs between the cells collected at the beginning of the process to those at the end using differentialGeneTest function with argument qval<0.01 in Monocle. The dimensions of the dataset were then reduced using the first two principal components with the “DDRTree” method. Next, we used orderCells function to order the cells based on the selected DEGs and the trajectory of the cells was visualized by the plot_cell_trajectory function in Monocle. The temporal expression of the gene of interests was visualized using the plot_genes_in_pseudotime function in Monocle. Additionally, to observe dynamic changes in the expression levels of the genes that were branch dependent (i.e., along with specific lineage), we used plot_genes_branched_heatmap function in Monocle to construct a special type of heatmap in which genes that had similar lineage-dependent expression patterns were clustered together.

WGCNA reconstruction of GRNs and hub genes. We used WGCNA, an algorithm implemented in the WGCNA R package, to reconstruct GRNs and to identify their associated hub genes that regulate cell differentiation. First, the dataset of interest (e.g., a given time point) created in Seurat was converted into a plain matrix for a given gene (in the column) in an individual cell (in a row). The dataset was then cleaned by removing cells with too many missing values using the goodSamplesGenes function in WGCNA. Next, we used the pickSoftThreshold function in WGCNA to determine the proper soft-thresholding power (β) that fits the criterion of the approximate scale-free topology of the network, and an adjacency matrix was then built with soft-thresholding power of eight in our study. Hierarchical clustering and GRN were constructed by using blockwiseModules function with arguments TOMType set to unsigned, networkType set to sign, and mergeCutHeight set to 0.25 in WGCNA. Modules containing genes that were highly associated with each other were identified in this process. Gene lists of interesting modules were extracted and submitted to DAVID for GO term analysis to retrieve their biological process and molecular functions. We then identified TFs and TF regulators from the genes based on the GO terms in molecular functions. We then selected the top 100 genes that had the highest weight (i.e., high correlation coefficient) connected to a given TF or TF regulator. Finally, the GRN based on these TFs and TF regulators then underwent cluster analysis using community cluster (GLay) and was then visualized using Cytoscape60. Hub genes for each GRN were identified as genes with high weight (summed correlation coefficients), high degree (summed connectivity, i.e., total numbers genes connected to this specific gene), and high betweenness centrality (BC) measure of the network. The hub gene of a given GRN was visualized by ComplexHeatmap R package.

Multicellular signaling and ligand-receptor models. To investigate the ligand-receptor interaction in heterogenous multicellular signaling systems, we used a list comprising 2557 human ligand-receptor pairs curated by Database of Ligand-Receptor Partners, IUPHAR, and Human Plasma Membrane Receptome. We first quantified the percentage of the cells (i.e., neural cells, melanocytes, and chondrocytes) that expressed a specific WNT ligand and its associated frizzled (FZD) receptors using scRNA-seq datasets. To ensure the ligand and receptors are uniquely expressed, we required that their expression in fold change needs to more than 0.25 on a natural log scale. We then used Circlize R package to visualize the directions of the signaling in the cell type based on connections of ligand-receptor pairs.

RNA fluorescence in situ hybridization (RNA-FISH). To validate scRNA-seq findings and to visualize the spatial distribution of WNTs and COL2A1 within pellets, we performed RNA-FISH for WNT3A, WNT4, and COL2A1 expression. d28 pellets with or without WNT-C59 treatment were harvested (n=3 time point) and snap-frozen in liquid nitrogen. Pellets were cryo-sectioned at 10 μm thick and fixed using 4% paraformaldehyde in PBS on ice for 10 min. Sample pre-treatment and RNA probe hybridization, amplification, and signal development were performed using the RNAscope Multiplex Fluorescent Reagent Kit v1 (Advanced Cell Diagnostics, #320850) following the manufacturer's instruction. Samples were imaged with multichannel confocal microscopy (Zeiss LSM 880). Tiled images with Z-stacks were taken at 20× magnification to capture the entire pellet. Maximum intensity projection, a process in which the brightest pixel (voxel) in each layer along Z direction is projected in the final 2D image, was performed using Zeiss Zen Blue (version 2.5).

FACS for progenitors. Cells at the Cp stage with the treatment of BMP4, a combination of BMP4 and WNT3A, or a combination of BMP4 and WNT-C59 were dissociated and resuspended in FACS Buffer (PBS−/− with 1% FBS and 1% penicillin/streptomycin/fungizone (P/S/F; Gibco) at approximately 40×10⁶ cells per ml. The cells were treated with Human Tru Stain FC XTM (BioLegend, #422302) for 10 min at room temperature. Approximately, 10,000 cells in 100 μl were used for each compensation. Cells were labeled with appropriate antibodies including their associated isotype control (FITC-CD45, #304006; PE/Cy7-CD146, #361008; PE-CD166, #343904, all from BioLegend). Cells were incubated for 30 min at 4° C. and washed with FACS buffer twice. Samples were resuspended in a sorting medium consisting of DMEM/F12 with 2% FBS, 2% P/S/F, 2% HEPES (Gibco), and DAPI (BioLegend, #422801) at 4×10⁶ cells per ml and filtered through a 40 μm cell strainer. Cells were stored on ice prior to sorting. Five microliters of all antibodies were used per million cells in 100 μl staining volume; 10 μl of Tru Stain FC XTM was used per million cells in 100 μl staining volume. DAPI was used at 3 μM. An Aria-II FACS machine was used to compensate for the color overlapping and to gate the samples. Data were analyzed using FlowJo software (version 10.5.3).

Histology. Pellets were collected in 10% neutral buffered formalin for fixation for 24 h. Pellets were then transferred to 70% ethanol, dehydrated, and embedded in paraffin wax. Pellet blocks were sectioned at 8 μm thickness and stained for proteoglycans and cell nuclei according to the Safranin-O and hematoxylin standard protocol.

Immunohistochemistry. Histologic sections (8 μm thick) of the pellets were rinsed with xylenes three times and rehydrated before labeling. Antigen retrieval was performed with 0.02% proteinase K for 3 min at 37° C. for COL2A1 and COL6A1 and with pepsin for 5 min at room temperature for COL1A1 and COL10A1 followed by peroxidase quench then serum blocking for 30 min at room temperature. Samples were labeled for 1 h with the primary antibody against COL1A1 (1:800 Abcam #90395), COL2A1 (1:10 Iowa #II-II6B3-s), COL6A1 (1:1000 Fitzgerald #70F-CR009X), and COL10A1 (1:200 Sigma #C7974) and for 30 min with the secondary antibody goat anti-mouse (1: 500, Abcam #97021) or goat anti-rabbit (1:500 Abcam #6720) as appropriate. Histostain Plus Kit (Sigma, #858943) was then used for enzyme conjugation for 20 min at room temperature followed by AEC (ThermoFisher, #001111) for 2.5 min (COL2A1 and COL6A1) or 2 min (COL1A1 and COL10A1) at RT. Finally, samples were counterstained with hematoxylin to reveal cell nuclei for 45 sec and mounted with Vector Hematoxylin QS (Vector lab, #H3404). Images were taken by the Olympus VS120 microscope (VS120-S6-W).

Biochemical analysis of cartilaginous matrix production. Pellets were rinsed with PBS after chondrogenic differentiation and digested at 65° C. overnight in 200 μl papain solution consisting of 125 μg ml⁻¹ papain (Sigma, P4762), 100 mM sodium phosphate, 5 mM EDTA, and 5 mM L-cysteine hydrochloride at 6.5 pH. Samples were stored at −80° C. before thawing to measure double-stranded DNA by Quant-iT PicoGreen dsDNA Assay Kit (ThermoFisher, #P11496) and glycosaminoglycans (GAG) by the 1,9-dimethylmethylene blue assay at 525 nm wavelength63. GAG content, as calculated based on the standard curve, was normalized to double-stranded DNA content to obtain the GAG/DNA ratio.

RT-qPCR. RNA of the pellets was isolated using the Total RNA Purification Kit according to the manufacture's protocol (Norgen Biotek, #37500). Reverse transcription of the RNA was performed using SuperScript VILO Master Mix (Thermo Fisher, #11755050). Fast SYBR Green Master Mix (Thermo Fisher, #4385614) was used for reverse transcription-quantitative polymerase chain reaction (RT-qPCR) according to the manufacturer's instructions on the QuantStudio 3 (Thermo Fisher). Gene expression was analyzed using the ΔΔCT method relative to undifferentiated hiPSCs with the reference gene TATA-box-binding protein (TBP).

Western blots. To examine the effect of WNT-C59 on WNT inhibition in the pellets at protein levels, Western blot analysis was performed on d28 pellets with or without WNT-C59 treatments. Six to eight pellets per experimental group were pooled and digested with 0.04% Type II collagenase solution in DMEM/F12 for 1 h. Cells were washed once with PBS and lysed in RIPA buffer (Cell Signaling Technology, #9806S) with protease inhibitor (ThermoFisher, #87786) and phosphatase inhibitor (Santa Cruz Biotechnology, #sc-45044). Protein concentration was measured using the BCA Assay (Pierce). Ten micrograms of proteins for each well were separated on 10% sodium dodecyl sulfate-polyacrylamide gel electrophoresis gels with prestained molecular weight markers (Bio-Rad, 161-0374) and transferred to a polyvinylidene fluoride (PVDF) membrane. The PVDF membrane blots were incubated overnight at 4° C. with the following primary antibodies: anti-WNT2B (1:350, Abcam, ab178418), anti-WNT3A (1:1000, Abcam, ab81614), anti-WNT4 (1:500, Abcam, ab91226), anti-WNTSB (1:500, Abcam, ab93134), anti-WNT7B (1:2000, Abcam, ab155313) and anti-GAPDH (1:30000, Proteintech 60004-1-Ig) for loading control, respectively. Affinity purified horseradish peroxidase (HRP)-linked goat anti-rabbit IgG secondary antibody (1:3000, Cell Signaling, #7074) or horse anti-mouse IgG secondary antibody (1:3000, Cell Signaling, #7076) was added and incubated for 45 min at room temperature. Immunoblots were imaged and analyzed using the iBright FL1000 Imaging System (Thermo Fisher). After the WNT proteins were imaged, the blots were then stripped by incubating with restore plus Western blot stripping buffer (ThermoFisher Scientific) at room temperature for 15 min. A full scan of all unprocessed Western blots is provided in the Source Data File.

Statistical analysis. All data were presented as mean±SEM. Analyses were performed using SPSS Statistics (version 25) with significance reported at the 95% confidence level. In the current study, the number of pellets per group or treatment condition is technical replicates, while the number of mice per group are biological replicates.

Data availability. We acquired RNA-seq datasets of human primary chondrocytes from a previously published study (NIH Gene Expression Omnibus (GEO) accession number GSE106292), in which embryonic hind limb bud chondrocytes (age: 6 weeks, n=2), adolescent knee chondrocytes (age: 17 weeks, n=2), adult knee chondrocytes (age: 18-60 years, n=2), and growth plate chondrocytes (age: 14 weeks, 15 weeks, and 18 weeks, n=1 per age). For the datasets obtained from the previously mentioned study, gene expression counts were averaged if there were more than two samples of the same age. We also harvested chondrocytes from human costal cartilage and performed bulk RNA-seq on these samples (age: ˜70 years, n=3). However, it was challenging to collect rib cartilage from young healthy donors; thus, aged 70-year-old costal cartilages were used. To compare the difference between the phenotypes of chondrocytes derived from hiPSCs and hMSCs, we also used bulk RNA-seq datasets of hMSC chondrogenesis from our recent study (GEO accession number GSE109503)47. For the present study, our bulk RNA-seq and scRNA-seq datasets are available on GEO accession number GSE160787, incorporated herein by reference in its entirety.

### Results

(i) Bulk RNA-Seq Indicates Successful Differentiation of hiPSCs.

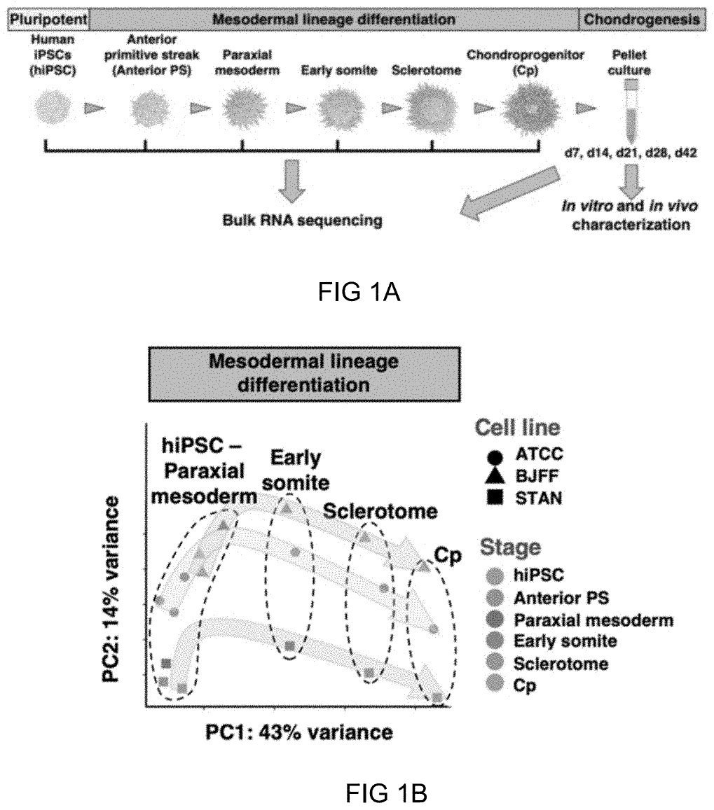

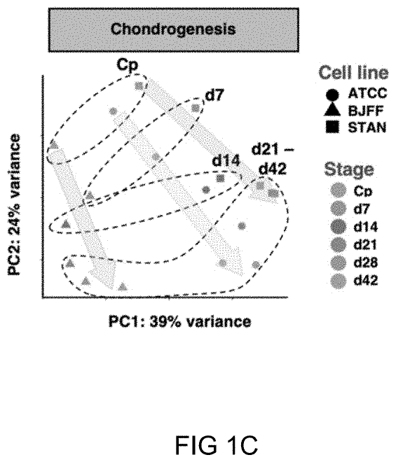

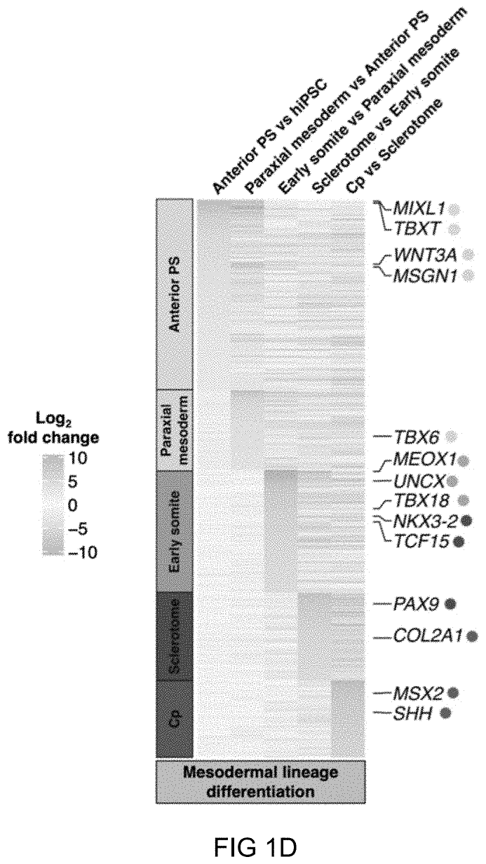

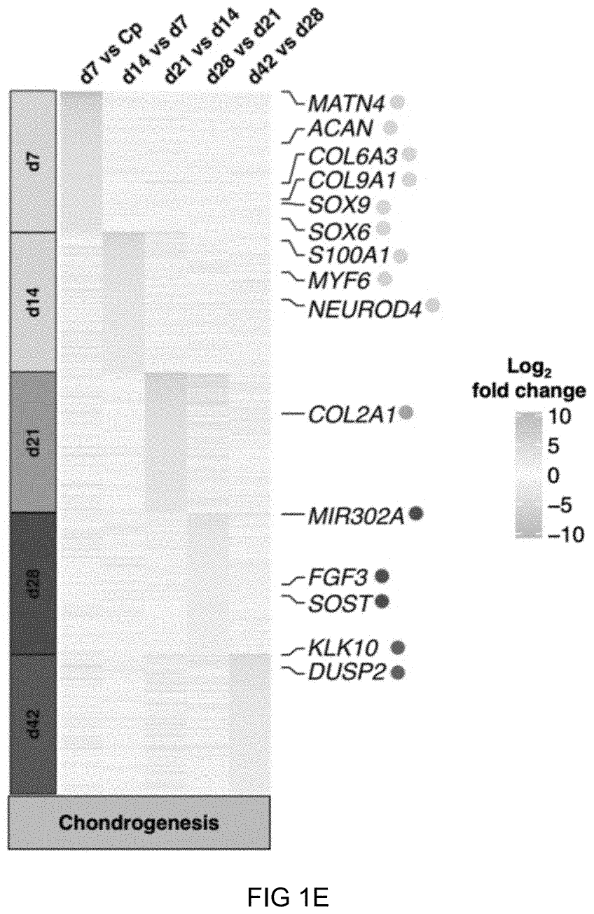

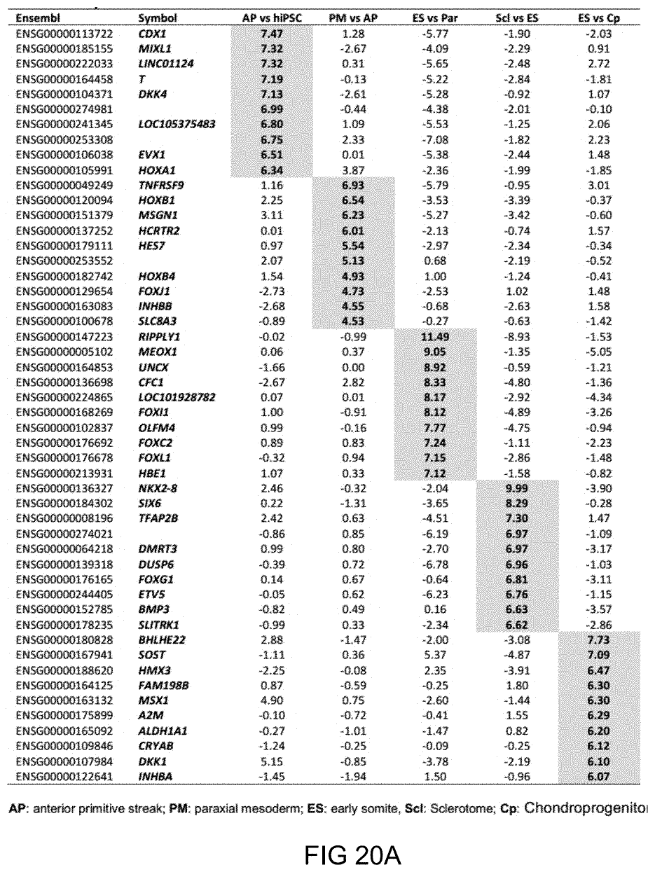

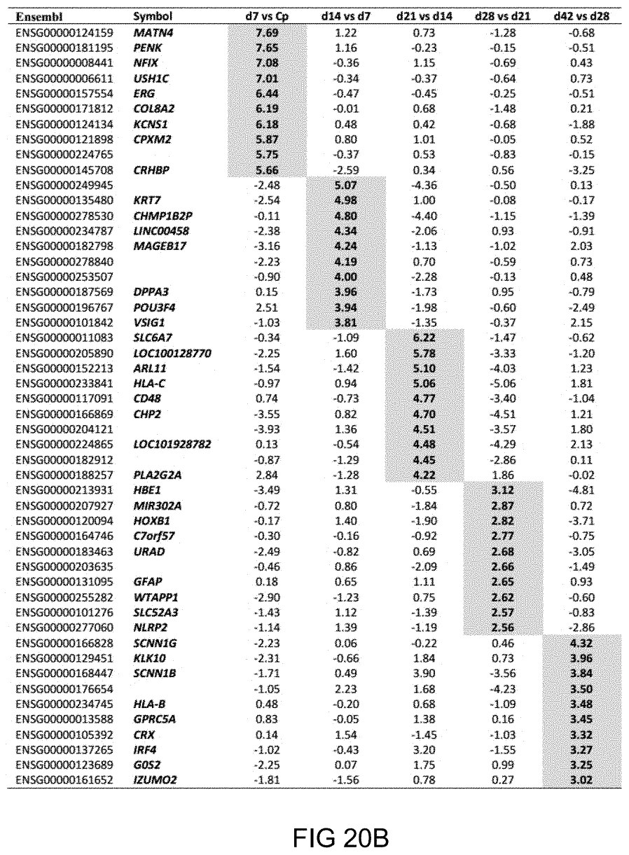

Previously, we reported a robust differentiation protocol that can drive hiPSCs toward a chondrogenic lineage via the paraxial mesoderm (FIG. 8A, 8B). To determine transcriptome profiles over the course of differentiation, three independent hiPSCs lines (ATCC, BJFF, and STAN) were collected for bulk RNA-seq at various stages (FIG. 1A). Principal component analysis (PCA) reveals that the three hiPSC lines follow similar mesodermal and chondrogenic differentiation trajectories (FIG. 1B, 1C). Analysis of differentially expressed genes (DEGs) between each stage revealed upregulation of stage-specific markers. For example, T-box transcription factor T (TBXT) and mix paired-like homeobox (MIXL1) were upregulated at the anterior primitive streak (anterior PS) stage compared to hiPSCs (FIG. 1D and FIG. 20A). Markers representing mesodermal derivatives including T-box 6 (TBX6), UNC homeobox (UNCX), and paired box 9 (PAX9) were upregulated sequentially at the stages of paraxial mesoderm, early somite, and sclerotome, respectively (FIG. 1D and FIG. 8C).

Chondrogenic markers such as matrilin 4 (MATN4), aggrecan (ACAN), collagen type VI alpha 3 chains (COL6A3), collagen type IX alpha 1 chain (COL9A1), and SRY-box 6 and 9 (SOX6 and SOX9) were upregulated as early as at day 7 (d7), while the expression of collagen type II alpha 1 chain (COL2A1) was increased at d21 (FIG. 1E and FIG. 20B). Interestingly, microRNA-302a (MIR302A), reportedly downregulated in osteoarthritic chondrocytes, had enhanced expression in d28 pellets. Neuronal differentiation 4 (NEUROD4), a gene encoding a transcriptional activator essential for neuronal differentiation, had increased expression in d14 pellets.

(ii) In Vitro Characterization of hiPSC-Derived Chondrocytes.

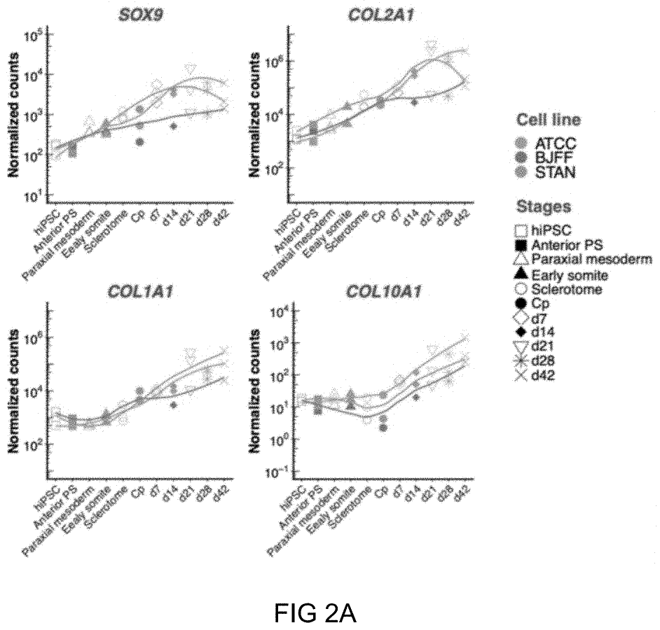

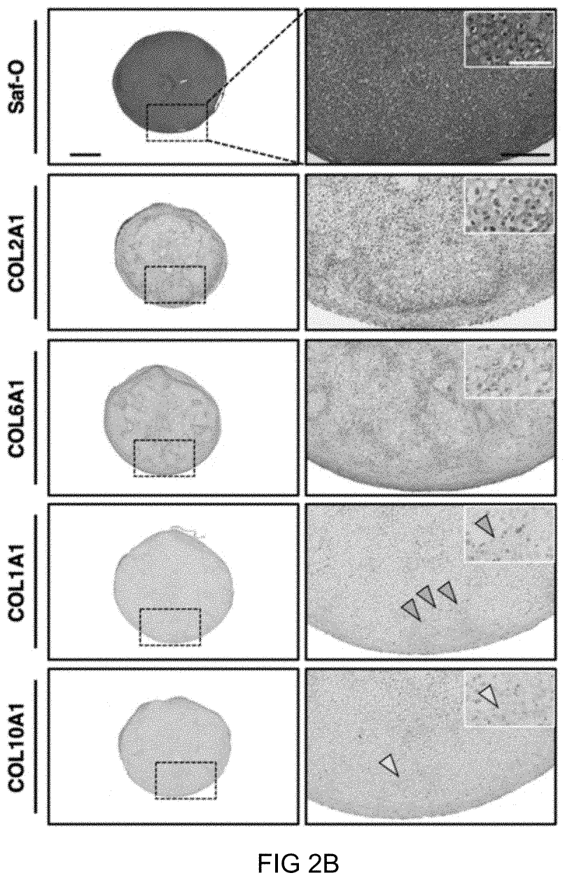

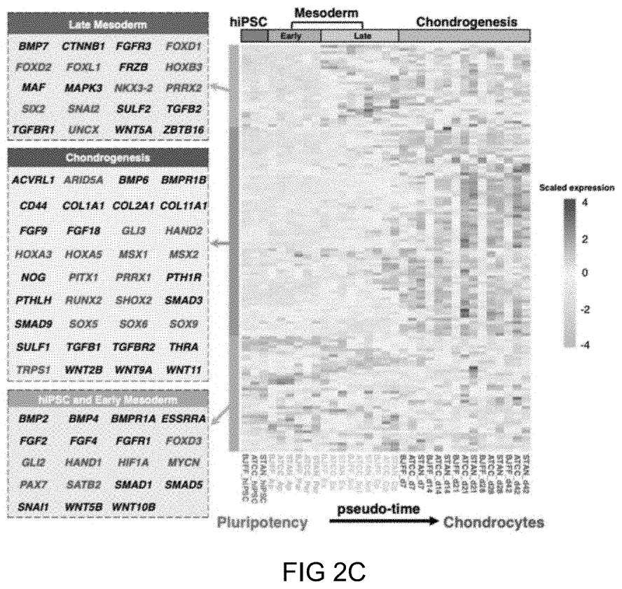

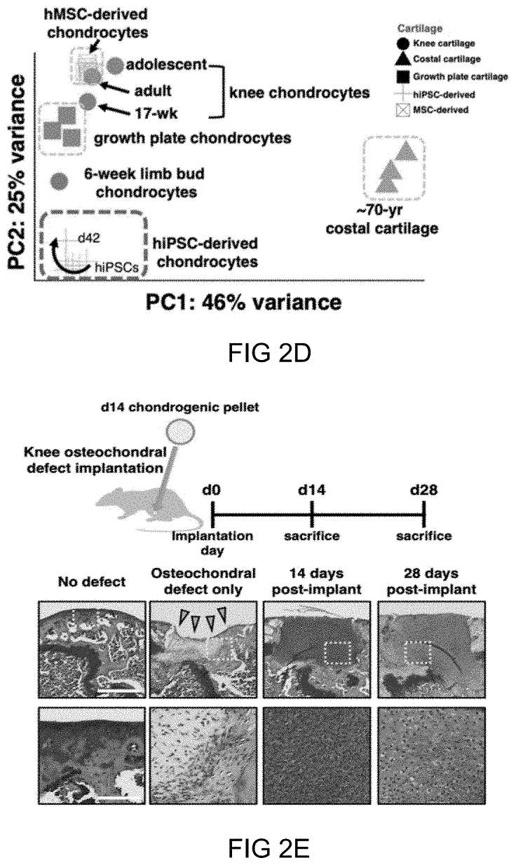

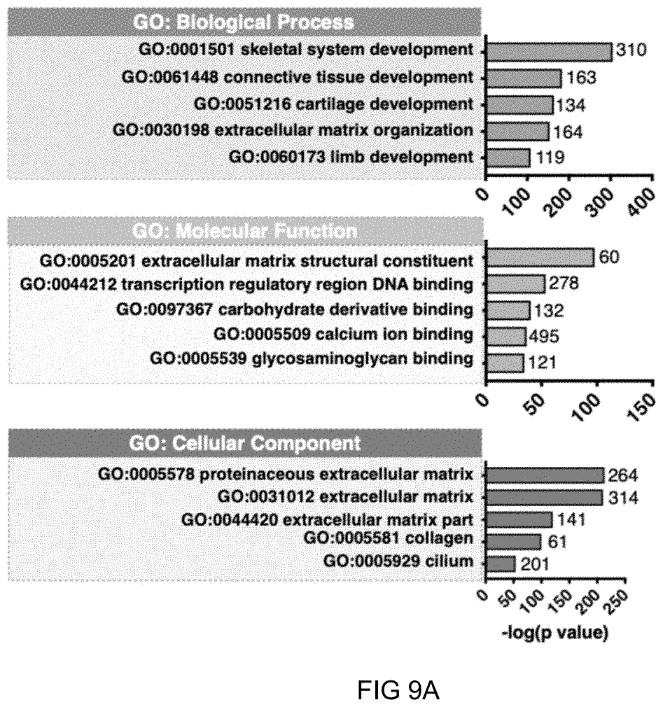

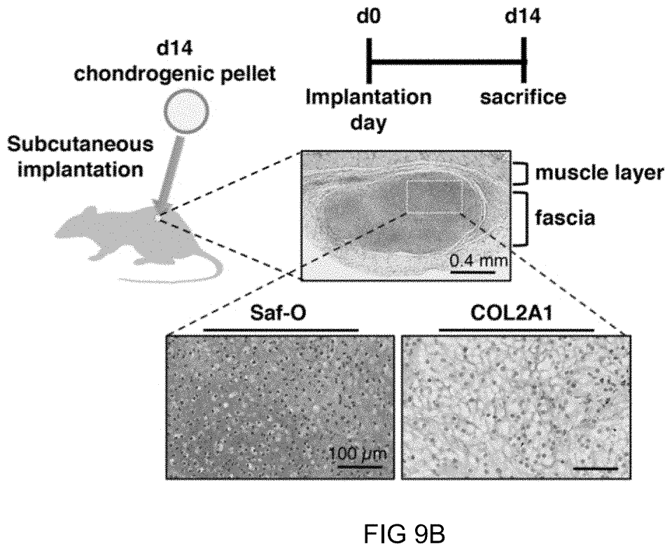

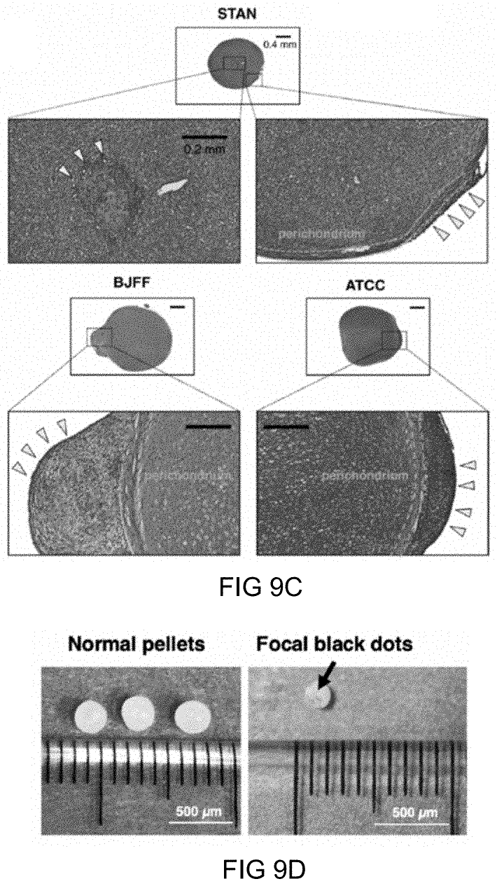

While temporal expression of chondrogenic markers such as SOX9 and COL2A1 were upregulated in unique hiPSC lines, both the hypertrophic chondrocyte marker collagen type X alpha 1 chain (COL10A1) and osteogenic marker collagen type I alpha 1 chain (COL1A1) also exhibited increased expression over time (FIG. 2A). It is important to note that COL1A1 is also a marker for fibrous tissues, perichondrium, and many other cell types. The d28 pellet matrix also demonstrated rich proteoglycan staining using Safranin-O (Saf-O) as well as intense labeling for COL2A1 and COL6A1 by immunohistochemistry (IHC). However, little labeling for COL10A1 and COL1A1 was observed despite increased gene expression of COL10A1 and COL1A1 at later time points (FIG. 2B). Gene ontology (GO) enrichment analysis of the genes using R package GAGE was performed. Significantly upregulated GO terms in Biological Process highlighted skeletal system and cartilage development (FIG. 9A). GAGE analysis also revealed that 134 out of the 205 genes defined by cartilage development (GO:0051216) were significantly increased. Interestingly, in addition to upregulated SOX5, 6, and 9, which are known to be master transcription factors (TFs) governing chondrogenesis, several WNTs, including WNT2B, had increased gene expression at different stages during differentiation (FIG. 2C).

To determine the phenotype of hiPSC-derived cartilage, bulk RNA-seq data and publicly available sequencing datasets of primary chondrocytes from a variety of cartilaginous tissues and chondrocytes derived from human mesenchymal stem cells (hMSCs) were projected in a PCA plot (FIG. 2D). It was found that hiPSC-derived chondrocytes demonstrated a similar phenotype to embryonic limb bud chondrocytes.

(iii) In Vivo Characterization of hiPSC-Derived Chondrocytes.

To determine whether hiPSC-derived chondrocytes could maintain their phenotype in vivo, d14 pellets were implanted subcutaneously in the dorsal region of immunodeficient NSG (NOD.Cg-Prkdc^{scid}IL2rg^{tm1WjI}/SzJ) mice (FIG. 9B). The d14 pellets represented the earliest time point when a chondrocyte-like phenotype was observed in vitro. After 14 days of implantation, pellets were harvested and found to retain a cartilage phenotype, with rich proteoglycan and COL2A1 labeling. No endochondral ossification was observed during this relatively short-term implantation period in this study.

To test whether hiPSC-derived chondrocytes can retain their phenotype within the joint, an osteochondral defect was created in the femoral groove of the mouse (FIG. 2E). Due to the small size of the mouse knee, the osteochondral defect model here also involves a growth plate defect. The defect was either left empty as a non-repair control group or filled with a d14 pellet. Defects left untreated did not exhibit any repair with hyaline cartilage, and only fibrotic tissue was observed. However, defects with pellet implantation demonstrated enhanced repair of the focal cartilage lesion, which was filled with cartilaginous matrix rich in Saf-O staining at both 14 and 28 days post implantation. This finding provides proof-of-concept of the maintenance of the chondrogenic phenotype over 28 days.

(iv) scRNA-Seq Mapping of Cellular Heterogeneity.

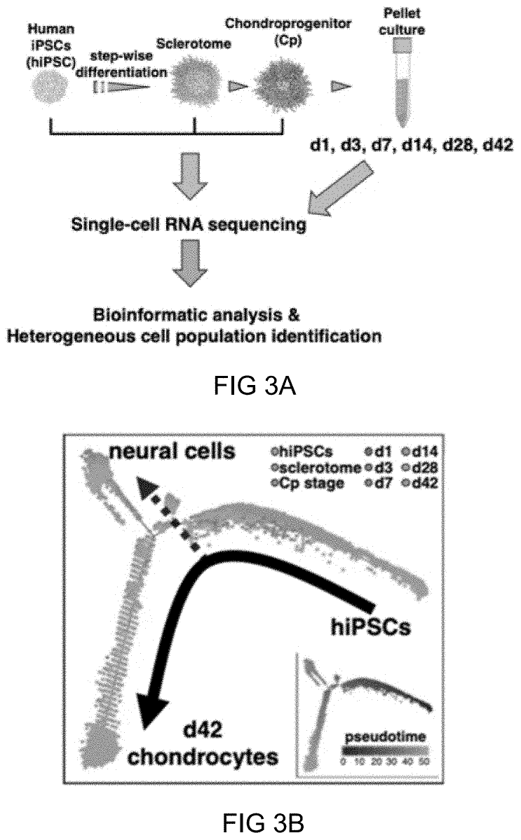

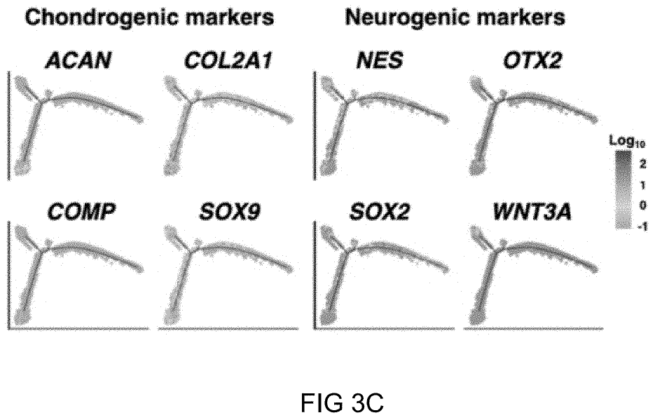

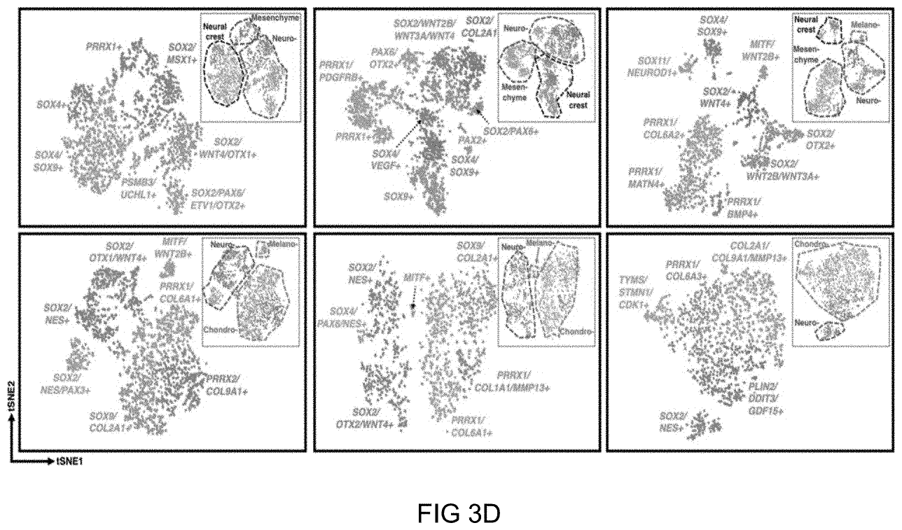

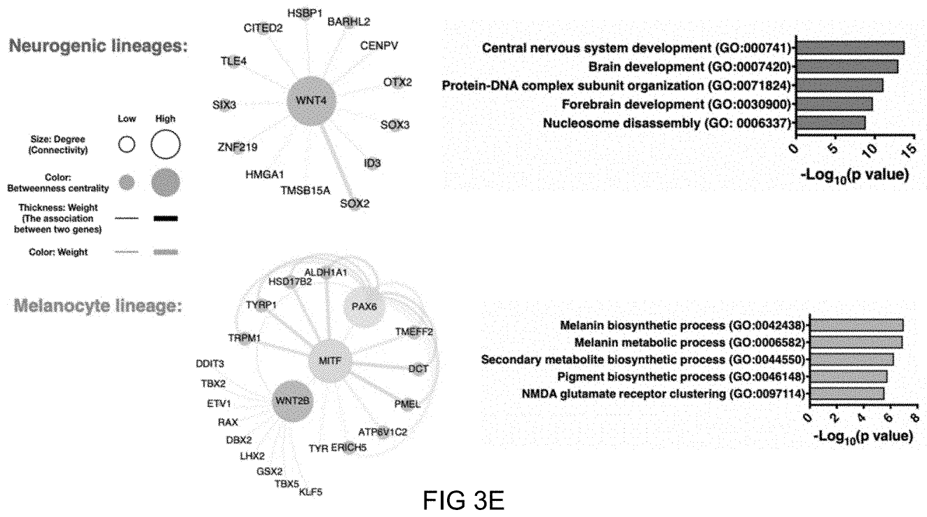

Although the protocol used generates a predominantly chondrocyte-like population as shown by IHC and bulk RNA-seq (FIG. 2B), it was often observed non-chondrocyte populations and occasional focal accumulation of black-pigmented regions on the surface of the pellets (FIG. 9C, 9D). These results suggest the presence of off-target differentiation, prompting us to seek their cellular identities. To dissect this cellular heterogeneity, nine samples from the STAN cell line at various differentiation time points were collected for scRNA-seq (FIG. 3A).

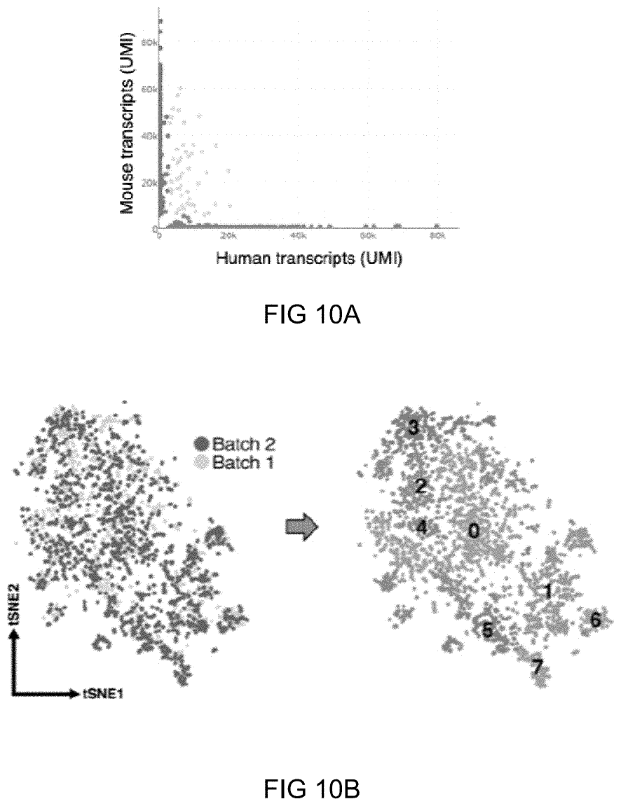

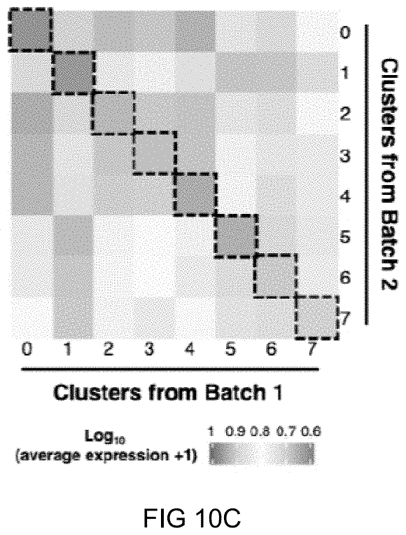

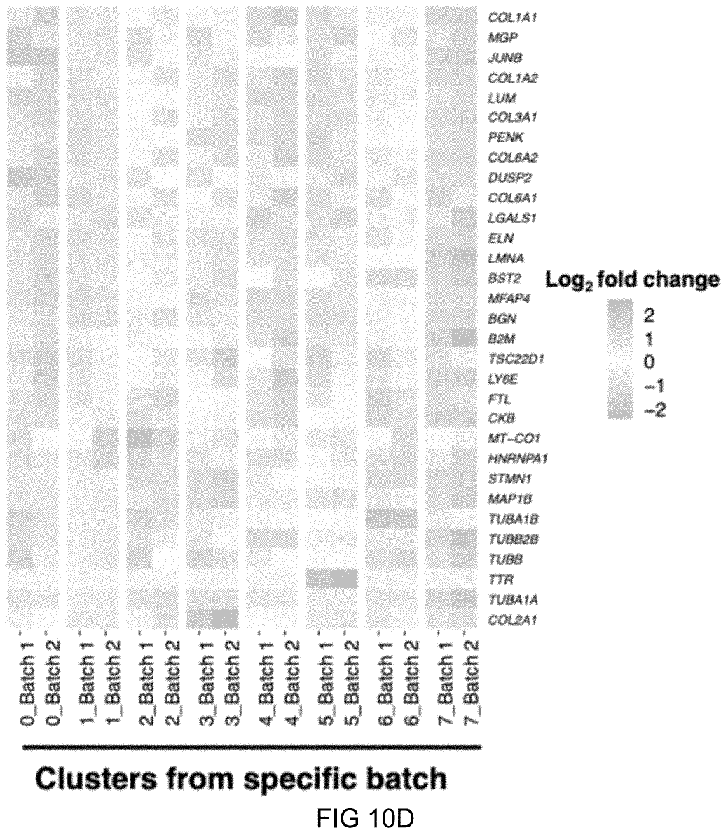

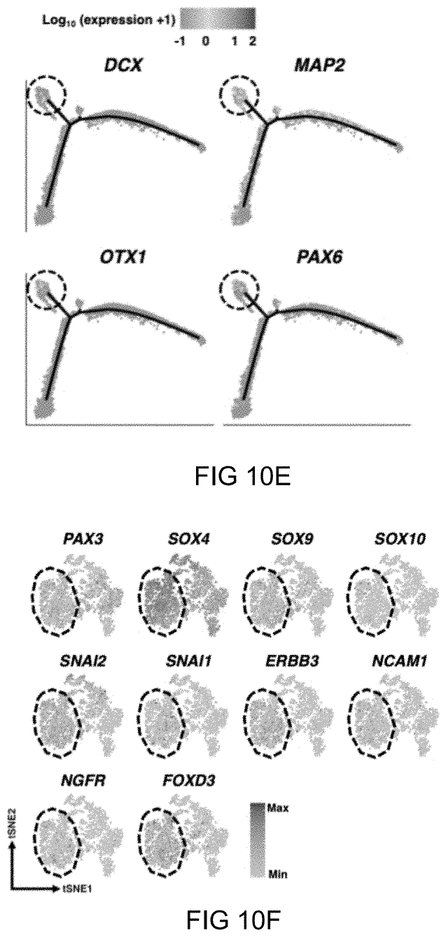

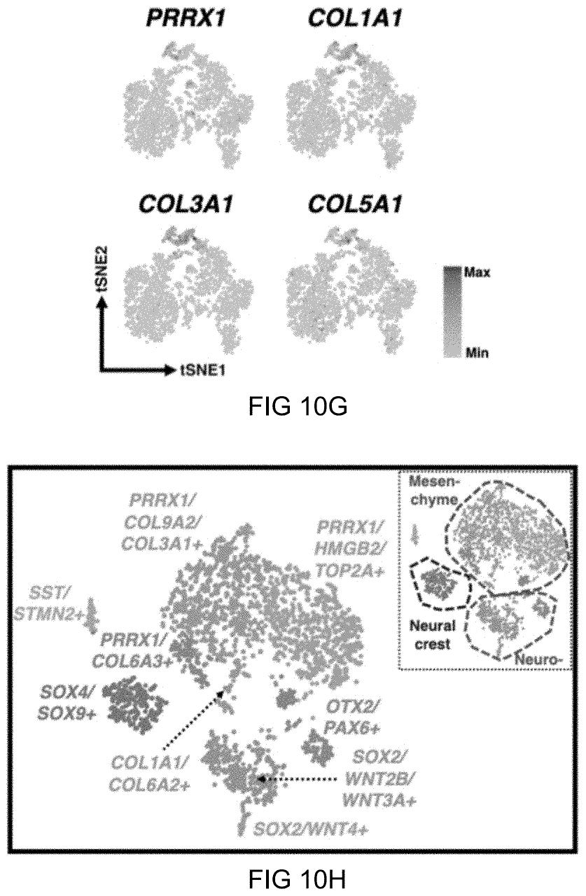

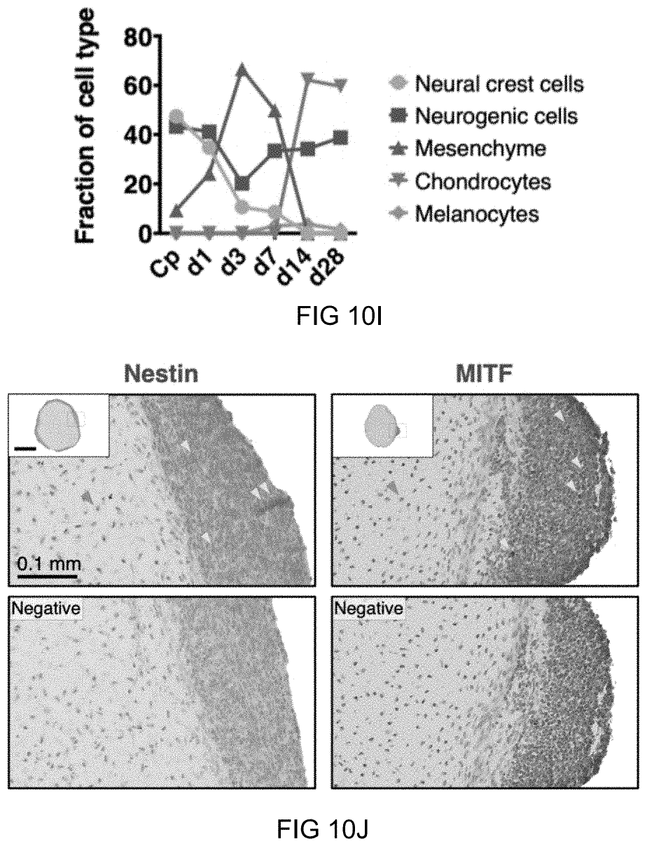

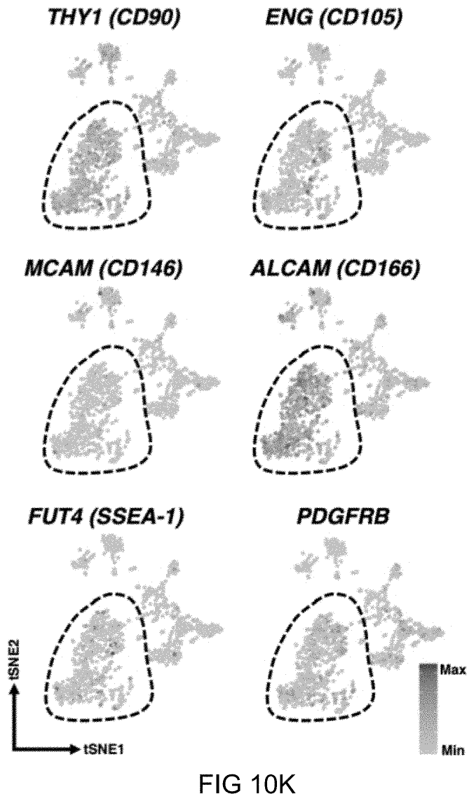

Sequencing of mixed-species ensured a low cell multiplet rate (2.7%) (FIG. 10A). To verify the reproducibility of the differentiation, two batches of d28 samples were collected from independent experiments for scRNA-seq. Canonical correlation analysis (CCA) was used to align cells from the two batches15 (FIG. 10B). The cells in the same cluster from different batches exhibited a high correlation in their gene expression (Spearman's rank coefficient rs>0.87 for all clusters) (FIG. 10C). Furthermore, genes that were highly conserved in one particular cluster showed similar expression patterns in the clusters from distinct batches, suggesting that our differentiation is highly reproducible (FIG. 10D).

(v) Lineage Bifurcation in hiPSC Differentiation Trajectory

We used the Monocle2 R package to reconstruct the differentiation trajectory from the stage of hiPSCs to d42 chondrocytes with a total of 19,195 cells that passed quality control (FIG. 3B). While cells following chondrogenic fate expressed chondrocyte markers, including ACAN, COL2A1, SOX9, and cartilage oligomeric matrix protein (COMP), one major branchpoint was found, diverting cell fate toward neural lineage with the expression of neural cell markers such as nestin (NES), orthodenticle homeobox 2 (OTX2), SOX2, and WNT3A (FIG. 3C). Other neural cell markers such as OTX1 and PAX6 were also enriched in this branch (FIG. 10E). The off-target cell differentiation toward neurogenic lineage confirmed our findings of increased NEUROD4 in the bulk RNA-seq data.

To explore distinct cell populations at each stage, scRNA-seq data were subjected to unsupervised clustering and visualized using t-distributed stochastic neighbor embedding (tSNE) plots (FIG. 3D). By comparing DEGs with signature genes of cell types in the literature and GO term analyses, broad cell populations were annotated by combining clusters expressing similar marker genes. For example, 2 of 7 clusters identified at the chondroprogenitor (Cp) stage not only had high expression levels of SOX4 and SOX9 but were also enriched in several markers resembling neural crest cells including PAX3 and forkhead box D3 (FOXD3) (FIG. 10F). Therefore, these two clusters were assigned to a broad cell population referred to as neural crest cells. Similarly, 4 clusters at the Cp stage exhibited markers of the neural lineage including SOX2, OTX1/2, and PAX6, and thus were annotated as neurogenic lineage cells, while PRRX1, COL1A1, and COL3A1 are known markers for mesenchyme (FIG. 10G). Similar major cell populations were also observed in dl and d3 pellets, and it appeared that the percentage of chondrogenic cells increased in d7 while there was a decreased percentage of neural crest cells over time (FIG. 10H, 10I).

Of note, a cluster with high expression of melanocyte-inducing TF (MITF) was observed in d7 and d14 pellets. MITF is a master TF regulating the development of melanocytes, cells that produce melanin (i.e., pigment). IHC of the pellets labeling for NES and MITF further confirmed the presence of neural cells and melanocytes (FIG. 10J), suggesting that the focal black dots observed at the surface of pellets are likely to be the pigment accumulation in melanocytes. Furthermore, mesenchymal cells in d14 pellets expressed several conventionally recognized MSC markers (FIG. 10K). Nevertheless, as distinct subtypes of hiPSC-derived chondrocytes and off-target cells were defined primarily based on marker genes, the complete functionality of these populations requires future investigation.

(vi) Lineage Bifurcation in hiPSC Differentiation Trajectory

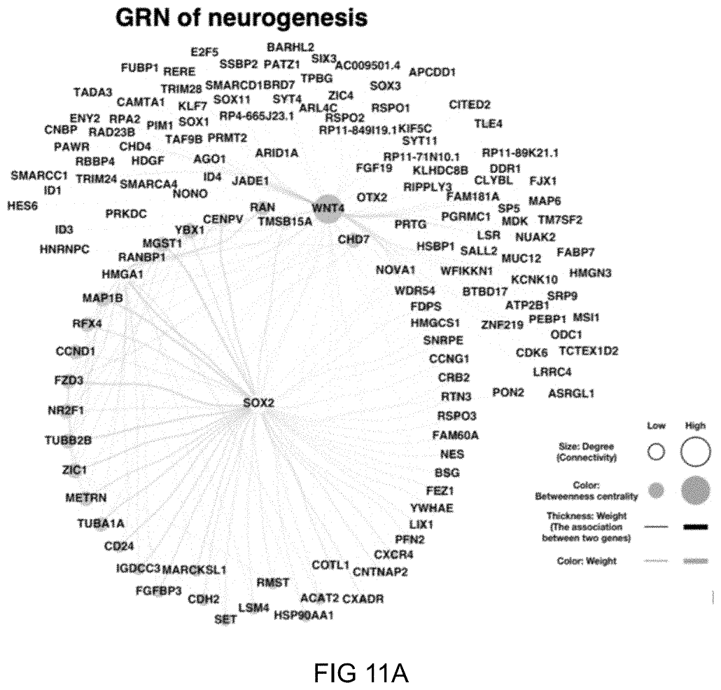

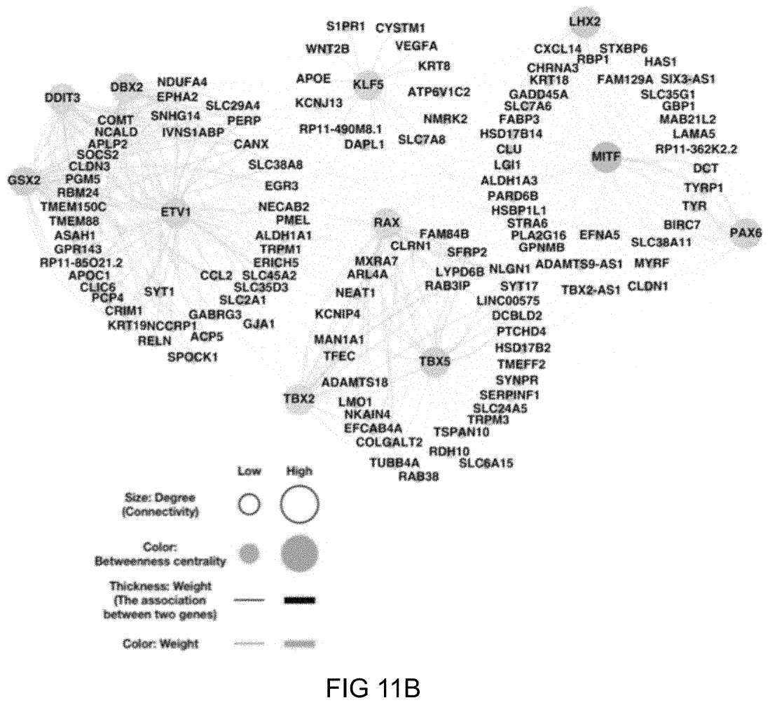

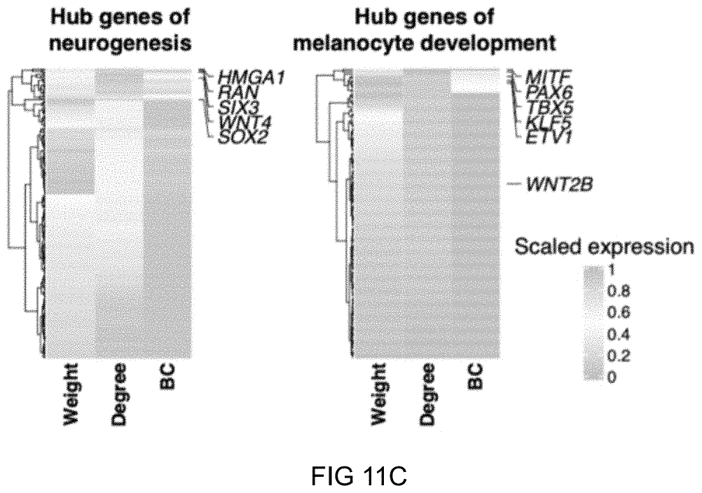

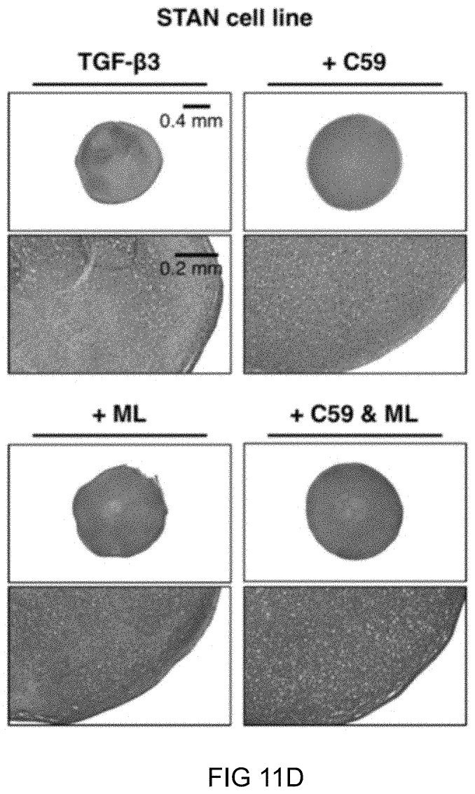

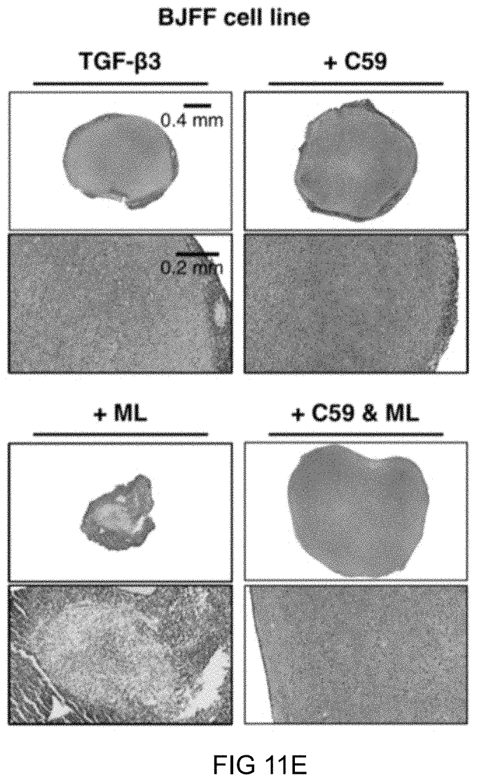

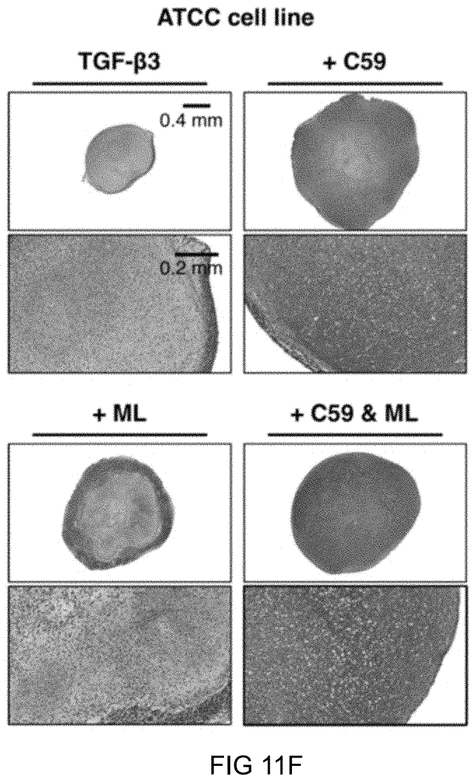

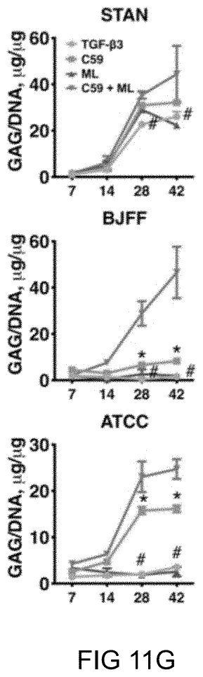

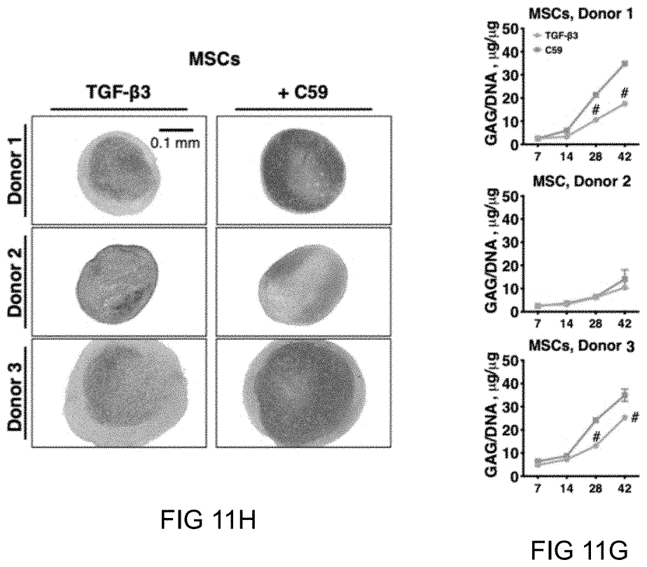

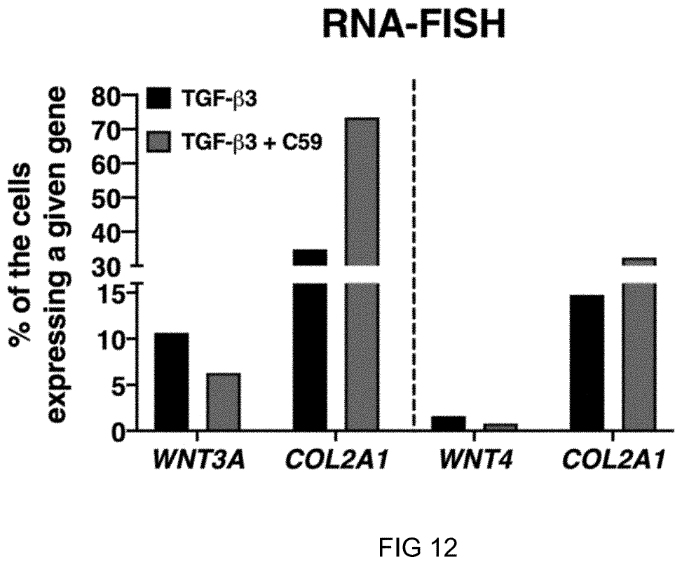

Next, we aimed to improve hiPSC chondrogenesis by decreasing off-target differentiation. Weighted gene co-expression network analysis (WGCNA) was performed to reconstruct GRNs and identify the hub genes that modulate neurogenesis and melanogenesis. scRNA-seq data of d14 pellets (with a total of 2148 cells and 3784 genes) were used for this computation due to the earliest presence of both chondrogenic and off-target populations detected. Five major gene modules (each containing >150 genes) were identified and based on GO enrichment analyses, they were categorized into cell division, cilium movement and assembly, skeletal system development, nervous system development, and melanin biosynthetic process. The genes in the modules of nervous system development and melanin biosynthetic process were then used to build corresponding GRNs and subnetworks by Cytoscape, while hub genes were determined by degree (node connectivity), weight (association between two genes), and betweenness centrality (BC) measure of the network (FIG. 3E and FIG. 11A-11C). In the GRN of neurogenesis, WNT4 was strongly associated with several TFs regulating neural differentiation. It was also observed that WNT2B was associated with both MITF and ETS variant 1 (ETV1), a gene whose activity has been reported to positively regulate MITF.

(vii) Inhibition of WNT Signaling Enhances hiPSC Chondrogenesis

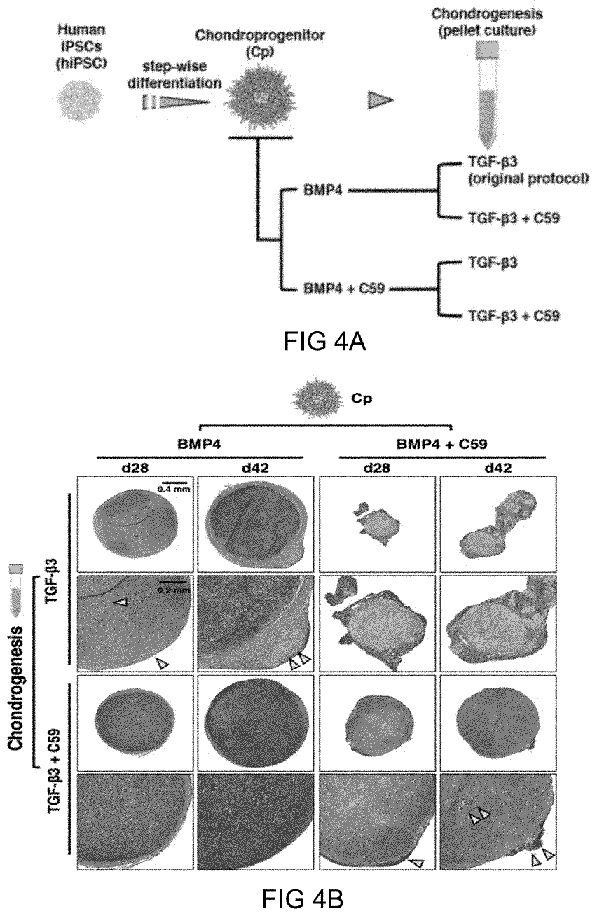

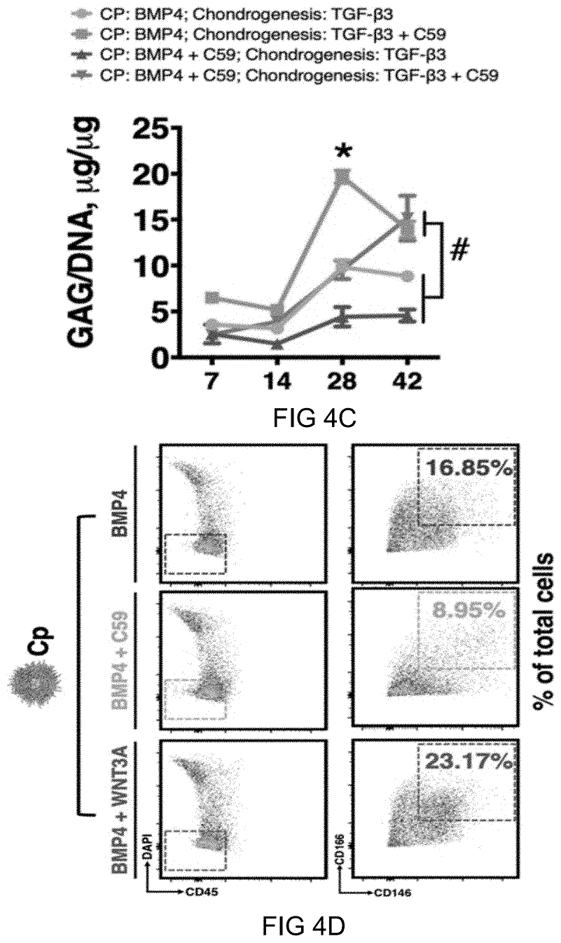

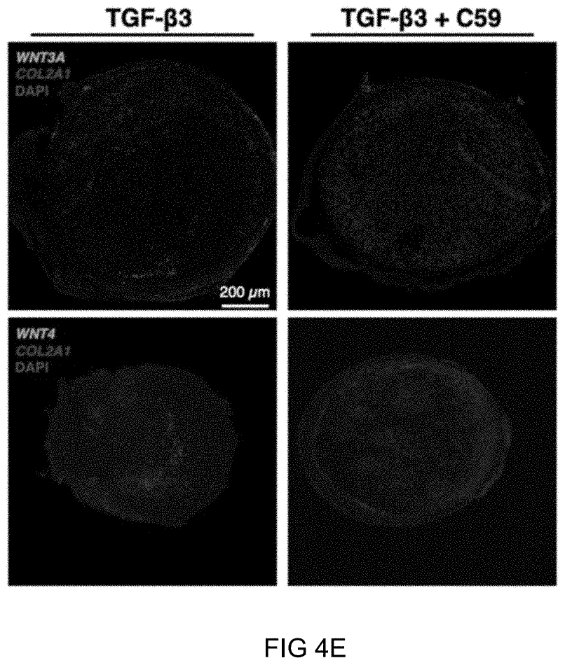

As WNTs were identified as essential genes in the off-target cells, it was hypothesized that inhibition of WNT signaling may improve hiPSC chondrogenesis by decreasing undesired cell populations. It is known that WNTs are required to properly specify somites from pluripotent cells. Therefore, WNT-C59, a WNT inhibitor, was administrated at either the Cp stage and/or during the chondrogenic pellet culture (i.e., four different inhibition regimens, FIG. 4A). Chondrocyte homogeneity, as indicated by Saf-O staining, was increased if WNT signaling was inhibited during pellet culture (FIG. 4B). This finding was reflected by the increased production of glycosaminoglycans per cell (GAG/DNA ratio) in the group receiving WNT-C59 during the pellet culture (FIG. 4C). However, inhibiting WNTs at the Cp stage severely impaired chondrogenesis. Mesenchymal cells that are positive for CD146 and CD166 are proposed to be putative Cps due to their robust chondrogenic potential. Flow cytometric analysis showed that WNT-C59 treatment largely decreased the percentage of CD146/CD166+ cells, while WNT3A supplementation increased this population at the Cp stage (FIG. 4D). Similar results were observed using two additional hiPSC lines (ATCC and BJFF) (FIG. 11D-11G). Interestingly, pellets derived from hMSCs with WNT inhibition also exhibited increased Saf-O staining (FIG. 11H, 11I). In addition, hiPSC pellets receiving combined administration of WNTC59 and ML329, an MITF antagonist, also exhibited enhanced chondrocyte homogeneity compared to standard TGF-β3 treatment (FIG. 11G).

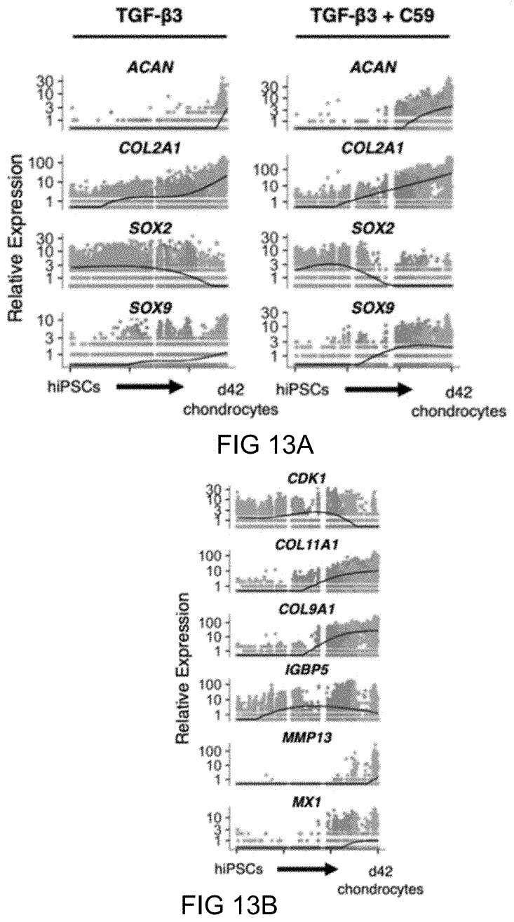

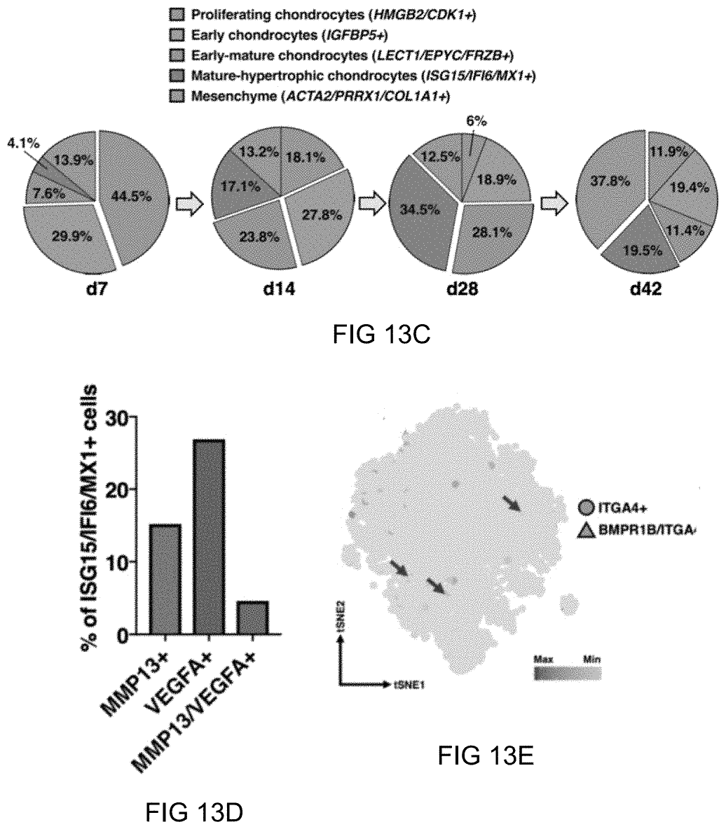

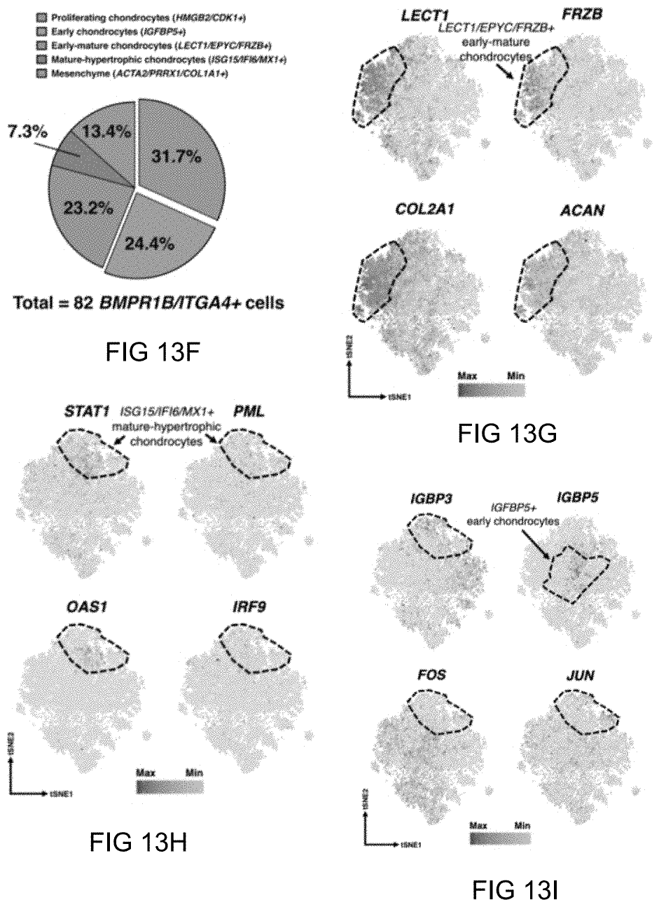

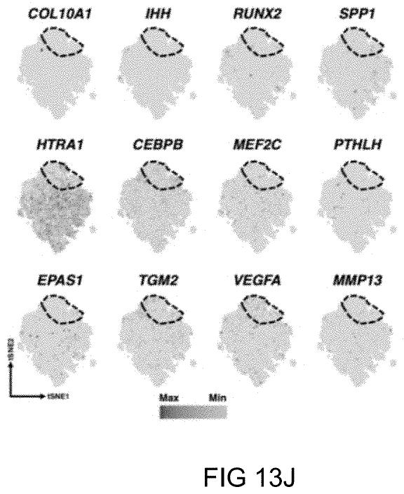

RNA fluorescence in situ hybridization (RNA-FISH) labeling of WNTs and COL2A1 within d28 pellets indicated that although some labeling could be detected in the center of the pellets, most WNTs were located in the perichondral layer, consistent to the inhomogeneous cell populations observed via IHC staining. Furthermore, WNT-C59-treated pellets showed a more homogenous distribution of COL2A1 RNA-FISH labeling vs. TGF-β3-treated pellets (FIG. 4E and FIG. 13).

(viii) scRNA-Seq Confirms WNT Inhibition Enhances Chondrogenesis

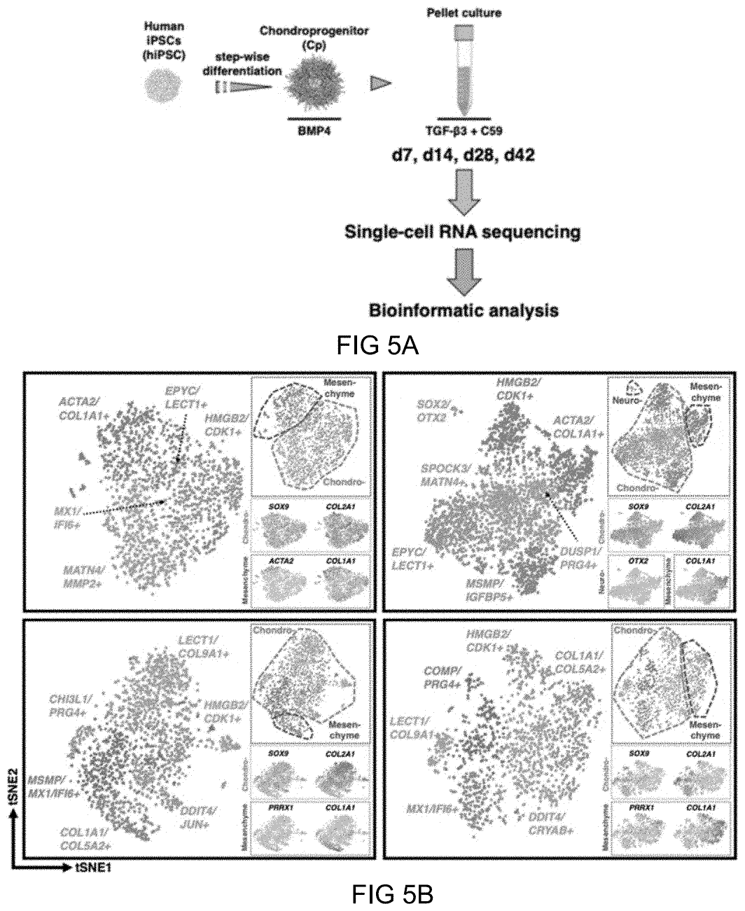

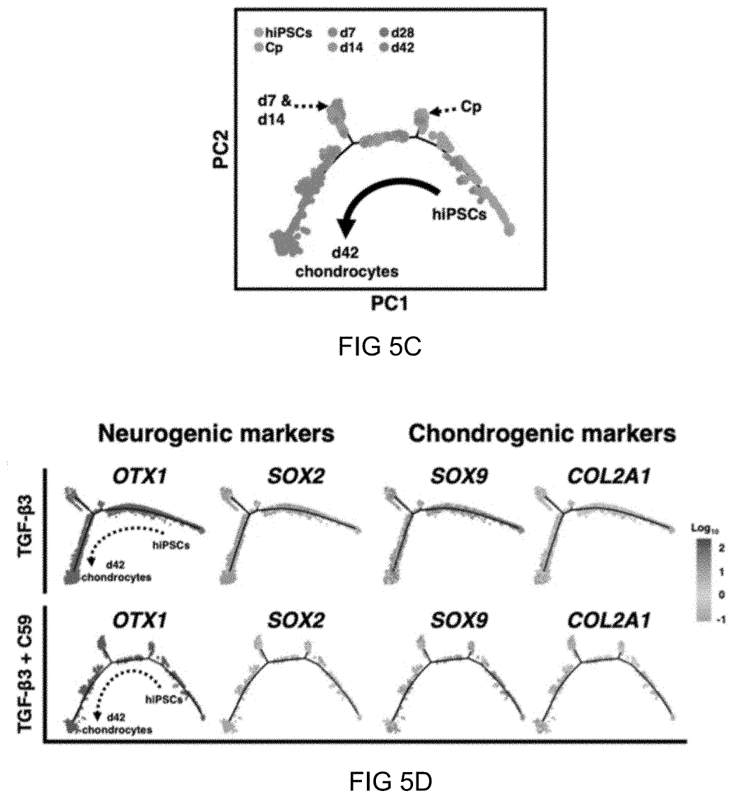

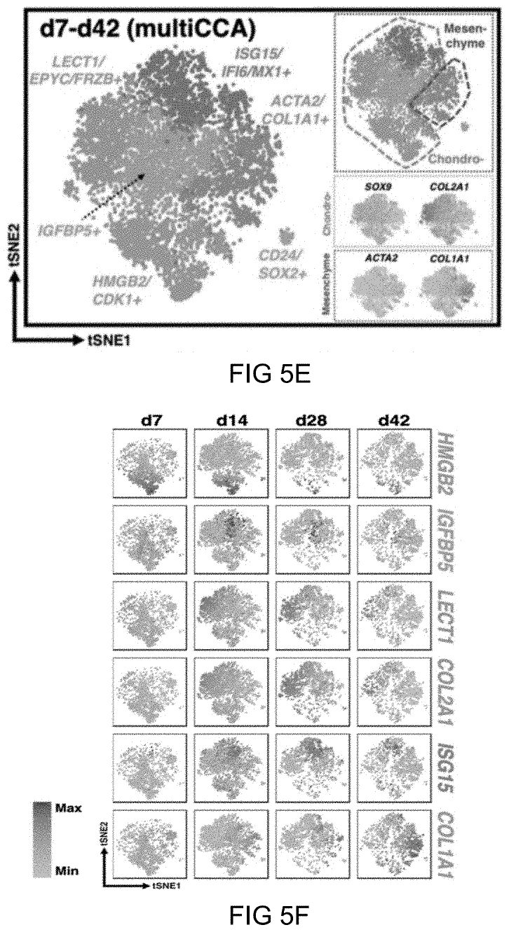

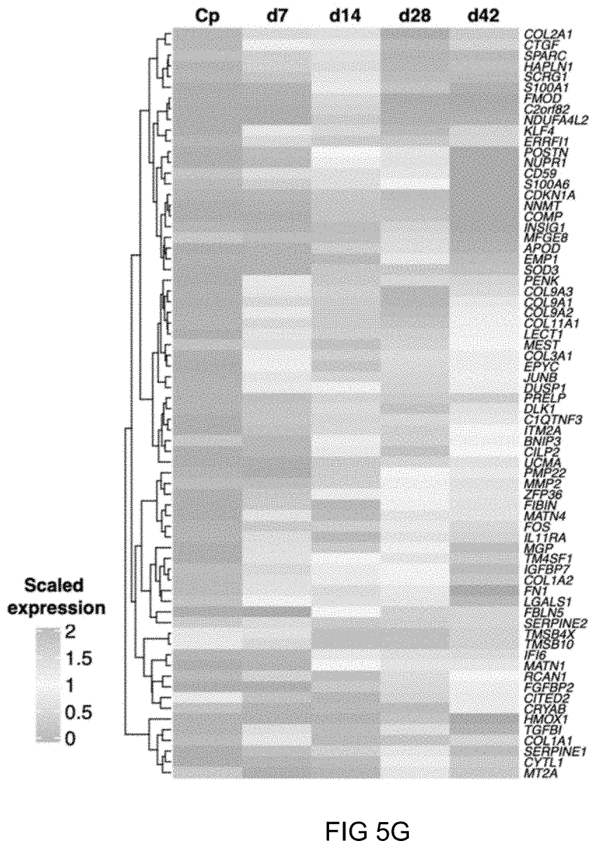

To determine how WNT inhibition altered cell populations in chondrogenesis and to identify chondrocyte subpopulations, pellets treated with WNT-C59 were analyzed using scRNA-seq with a total of 14,683 cells from the stage of hiPSC, Cp as well as d7, d14, d28, and d42 WNT-C59-treated pellets (FIG. 5A, 5B). It was found the WNT-C59-treated pellets comprised two major cell populations: mesenchyme and chondrocytes. Mesenchyme exhibited high expression of actin (ACTA2), PRRX1, COL1A1, and COL3A1. Most importantly, neural cells and melanocytes were significantly decreased with WNT inhibition. The differentiation trajectory of WNT-C59-treated chondrogenesis was reconstructed, using scRNA-seq datasets of hiPSC and Cp stages from the previous sequencing (since they did not involve WNT-C59 intervention) (FIG. 5C). Compared to the trajectory built from TGF-β3-treated pellets, WNT-C59-treated pellets exhibited little, if any, neurogenic markers, but showed enriched expression for chondrogenic markers (FIG. 5D). In pseudotime analysis, it was found that WNT-C59-treatment led to earlier induction of ACAN expression, higher levels of COL2A1 and SOX9 expression, and an earlier decrease in SOX2 expression as compared to pellets treated with TGF-β3 alone (FIG. 13A).

Chondrocytes in WNT-C59-treated pellets comprised several subpopulations as identified by multiple CCA alignment of d7-d42 timepoints with a total of 7997 cells (FIG. 5E, 5F, and FIG. 13B, 13C), including one mesenchymal population and four conserved chondrocyte subsets with enriched COL2A1 and SOX9 expression. The chondrocyte subset enriched in cell cycling markers, such as high mobility group box 2 and cyclin-dependent kinase 1 (HMGB2/CDK1⁺), was defined as proliferating chondrocytes. The second chondrocyte subset was enriched in IGF-binding protein-5 (IGFBP5). It has been previously reported that IGFBP5 is highly upregulated in the early differentiating stage. Hence, the IGFBP5⁺ chondrocyte subset was defined as a population of early differentiating chondrocytes. The third chondrocyte subset expressed leukocyte cell-derived chemotaxin 1, epiphycan, and frizzled-related protein (LECT1/EPYC/FRZB⁺) and had the highest levels of COL2A1 and ACAN expression among other chondrocyte subsets. Therefore, the LECT1/EPYC/FRZB⁺ chondrocyte subset was defined as a population of early mature chondrocytes. Finally, it was identified a unique chondrocyte subset expressing interferon (IFN)-related genes including ISG15 ubiquitin-like modifier, interferon-alpha inducible protein 6, and MX dynamin-like GTPase 1 (ISG15/IFI6/MX1+). It was observed that 4.6% of ISG15/IFI6/MX1⁺ chondrocytes co-expressed terminal hypertrophic differentiation markers VEGFA and MMP13; thus, the ISG15/IFI6/MX1⁺ chondrocyte subset were defined as mature-hypertrophic chondrocytes (FIG. 13D).

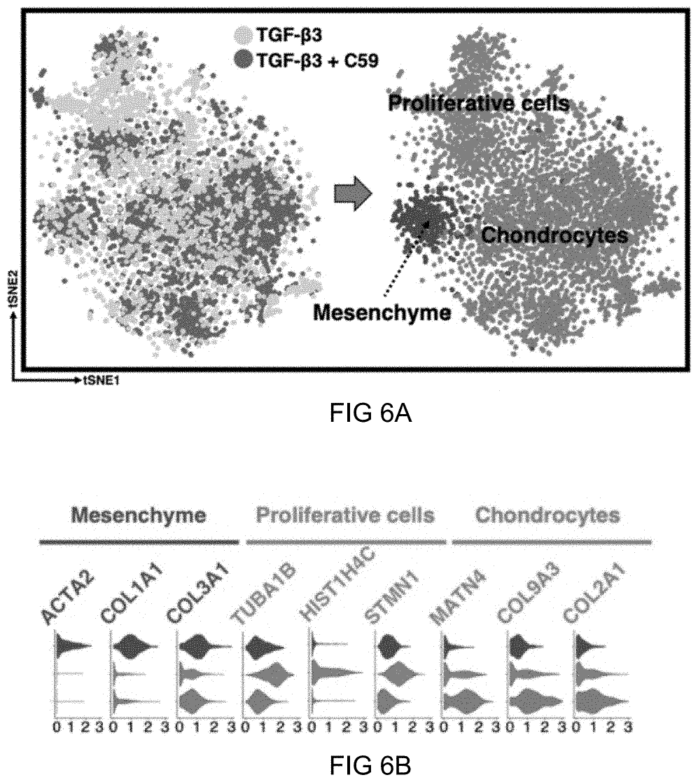

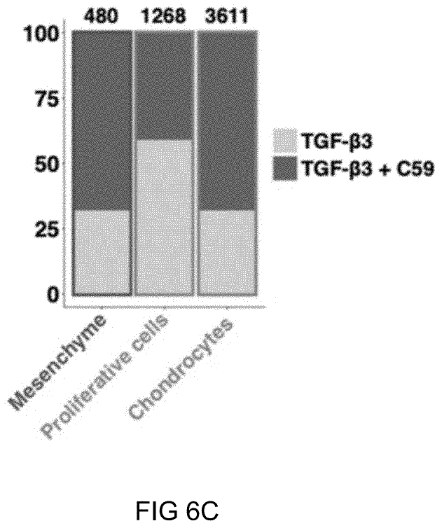

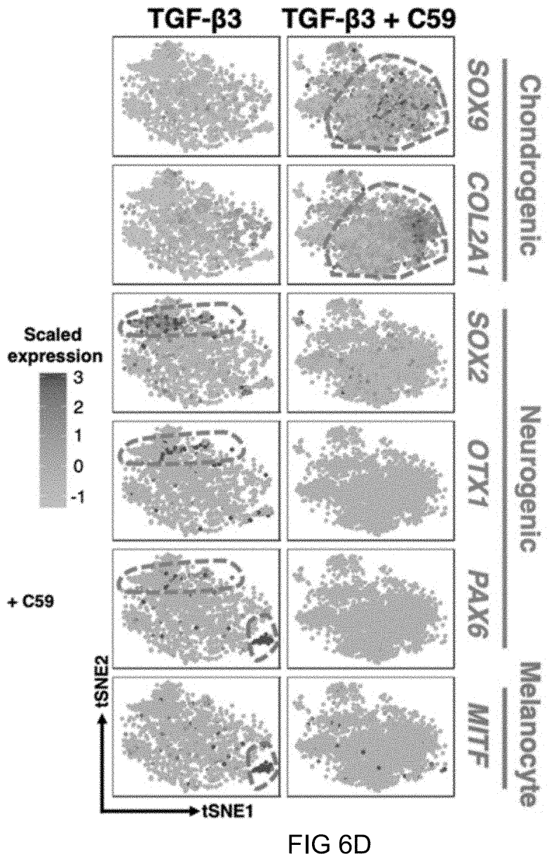

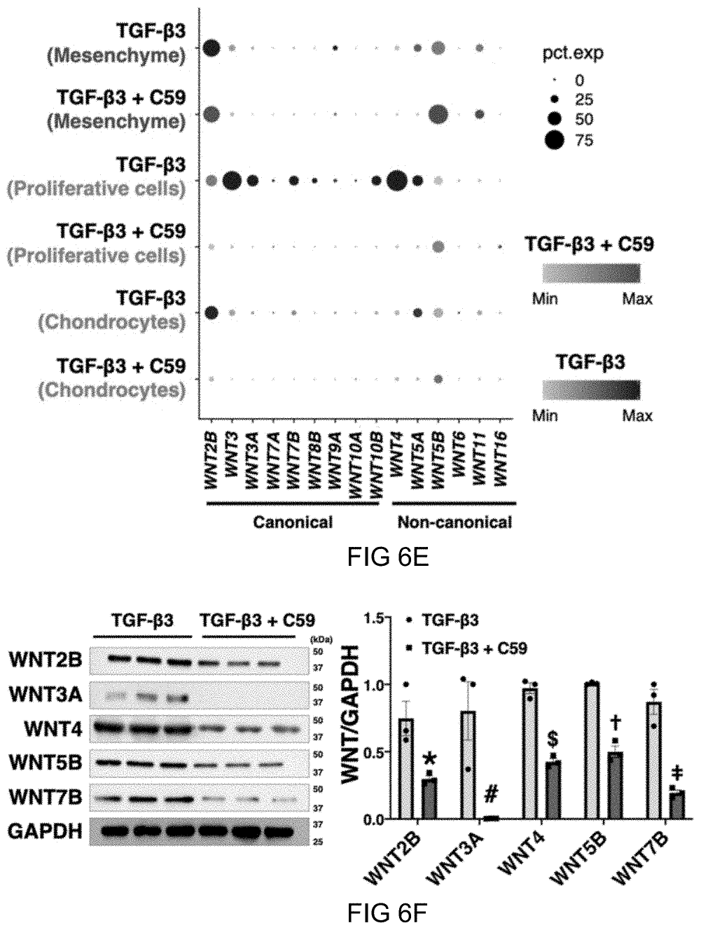

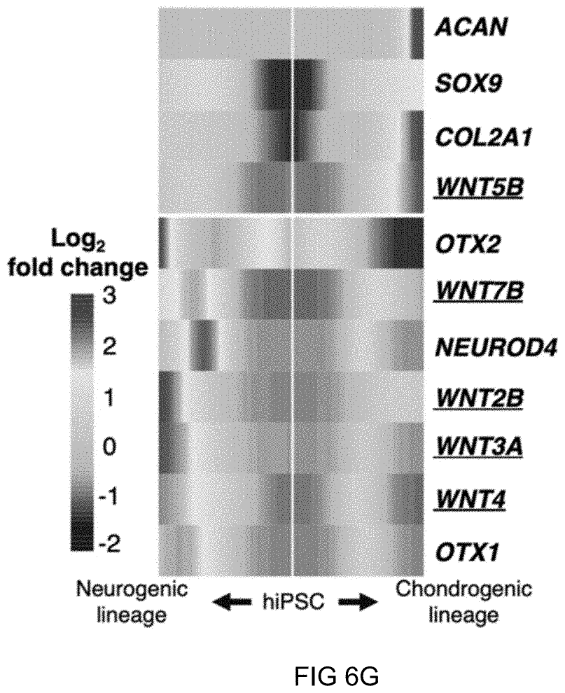

At early timepoint d7, HMGB2/CDK1⁺ proliferating chondrocytes was the main cell population (44.5%) within the pellets (FIG. 6C). Interestingly, this population also had the highest numbers of BMPR1B/ITGA4 double-positive cells, a rare osteochondral progenitor population found in articular cartilage (FIG. 13E, 13F). When proliferating chondrocytes differentiated toward maturity, potentially facilitated by IGFBP5, IGFBP5⁺ early differentiating chondrocytes and LECT1/EPYC/FRZB⁺ early mature chondrocytes became dominant (FIG. 13C). The enriched expression of FRZB, which encodes a secretory WNT inhibitor, in early mature chondrocytes might help stabilize this population by further antagonizing WNT signaling in addition to WNT-C59 treatment (FIG. 13G). As LECT1/EPYC/FRZB⁺ chondrocytes had the highest levels of COL2A1 and ACAN expression, the DEGs of this particular population were investigated at various time points (FIG. 5G). Among several early chondrogenic markers and osteogenic markers, COL1A2 and IGFBP7 exhibited biphasic upregulation at both early and later time points of chondrogenesis.

The percentage of ISG15/IFI6/MX1⁺ mature-hypertrophic chondrocytes greatly increased at d28 (FIG. 13C). Although the downstream IFN regulatory molecules including STAT1 and PML were elevated in this population, any type of IFNs were not detect which were conventionally believed to be the activators of IFN pathways (FIG. 13H). Instead, it was observed that IGFBP3 was enriched in ISG15/IFI6/MX1⁺ chondrocytes, whereas IGFBP5 was highly expressed in early differentiating chondrocytes. In line with the results of previous studies, it was also observed that IGFBP3 inhibited expression of FOS (C-FOS), a possible driver of chondrocyte hypertrophy when it dimerizes with JUN (AP-1) (FIG. 13I). This result may provide some explanations for the finding that ISG15/IFI6/MX1⁺ chondrocytes had variable expression levels of hypertrophic chondrocyte markers (FIG. 13J)

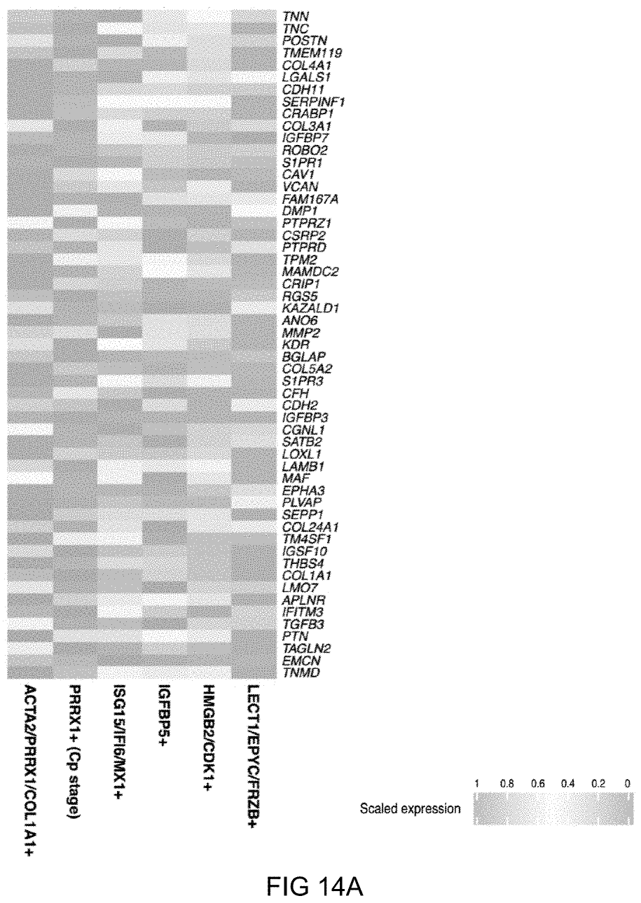

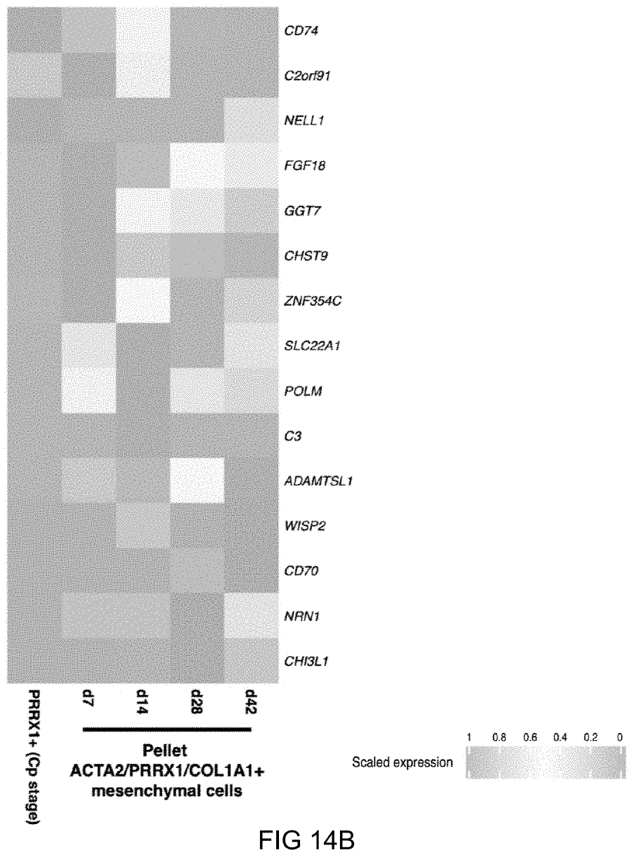

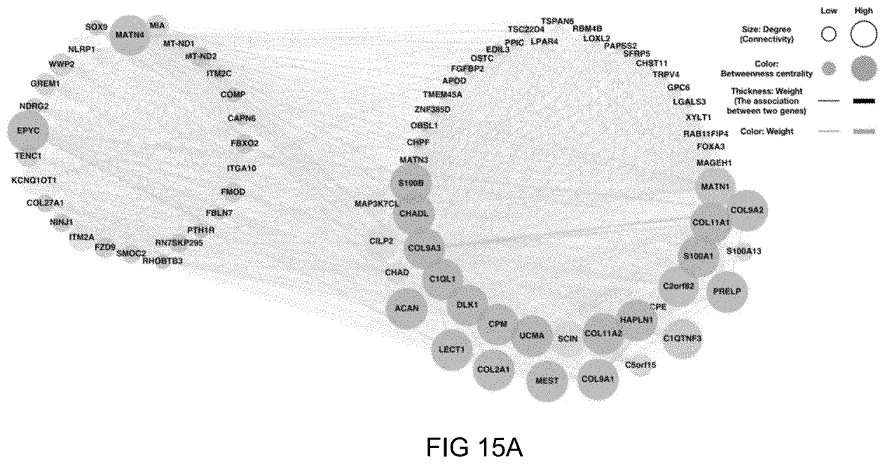

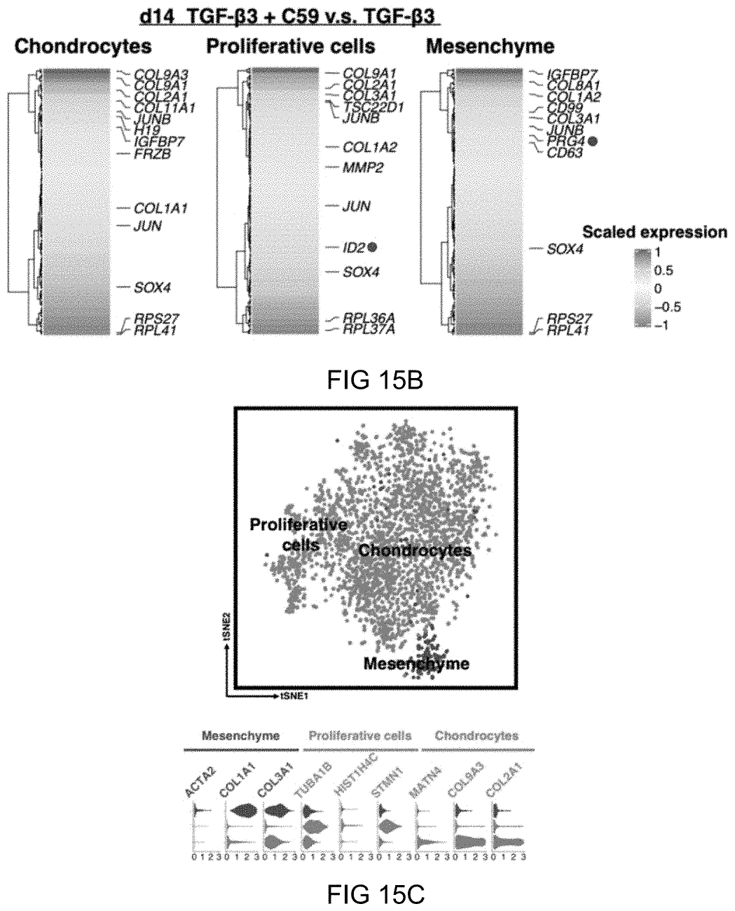

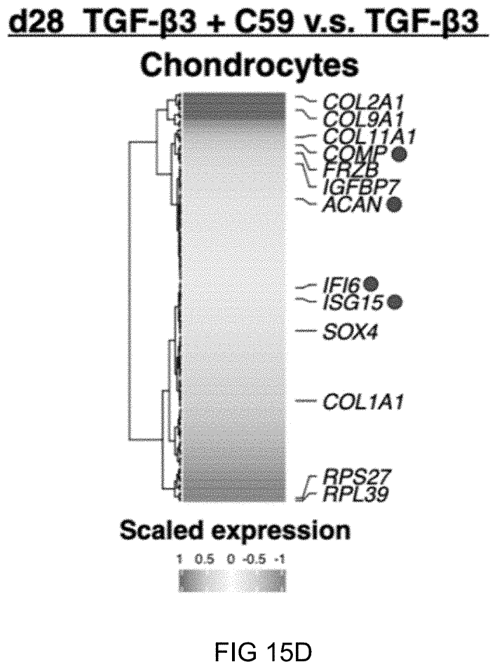

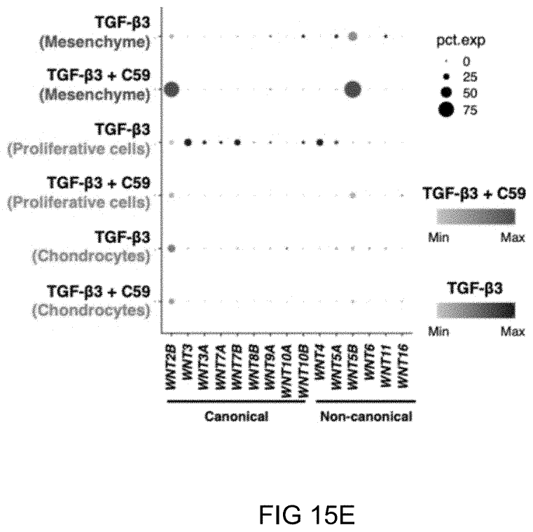

During chondrogenic culture, pellets were generally surrounded by a fibrous layer, resembling the cartilage anlage enclosed by fibroblastic cells (i.e., perichondrium). To determine if the mesenchyme (i.e., ACTA2/PRRX1/COL1A1⁺ cells) identified in pellets and the mesenchyme (i.e., PRRX1⁺ cells) identified at the Cp stage (monolayer culture) were similar to the perichondrium, these mesenchymal cells were benchmarked, as well as various chondrocyte subpopulations, against previously reported markers of perichondrial cells in rats and humans (FIG. 14). I was found that ACTA2/PRRX1/COL1A1⁺ cells in pellets, but not PRRX1⁺ cells at the Cp stage, were enriched in genes of perichondrium, suggesting that the mesenchymal population at the Cp stage and the mesenchymal population in pellets had distinct phenotypes, despite their shared mesenchymal genes such as COL1A1 and COL3A1. The scRNA-seq data of WNT-C59-treated pellets were then used to reconstruct the GRN of hiPSC chondrogenesis with minimal presence of off-target cells as shown by WGCNA (FIG. 15A).

(iix) Differential Gene Expression Profiles after WNT-C59 Treatment

Three major conserved populations were identified after CCA alignment of the d14 cells with or without WNT-C59 treatment (a total of 5224 cells analyzed): proliferative cells, mesenchyme enriched, and chondrocytes (FIG. 6A, 6B). WNT-C59-treated pellets contained more mesenchyme and chondrocytes at d14, while non-WNT-C59-treated (i.e., TGF-β3 only) pellets had more proliferative cells at the same time point (FIG. 6C). Pellets with only TGF-β3 treatment not only showed elevated expression of MITF but also had more neural cells which were clustered in proliferative cells (FIG. 6D). Chondrocytes and proliferative cells exhibited similar profiles of upregulated and downregulated DEGs. For instance, both cell populations showed upregulated expression of COL2A1 and JUNB, while exhibiting decreased expression of SOX4 and several ribosomal genes (FIG. 8B). Interestingly, FRZB was only upregulated in the chondrocyte population upon WNT-C59 treatment.

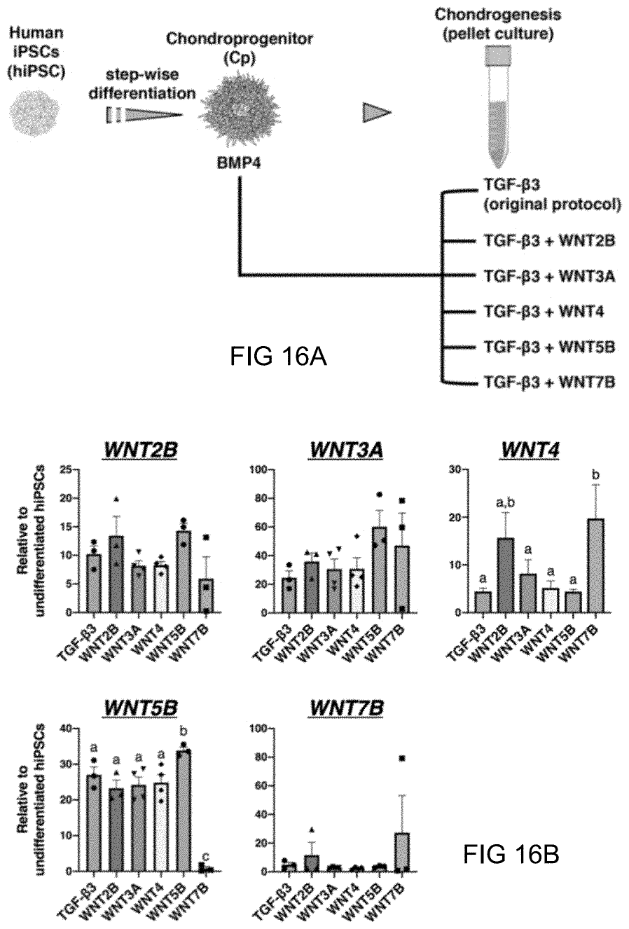

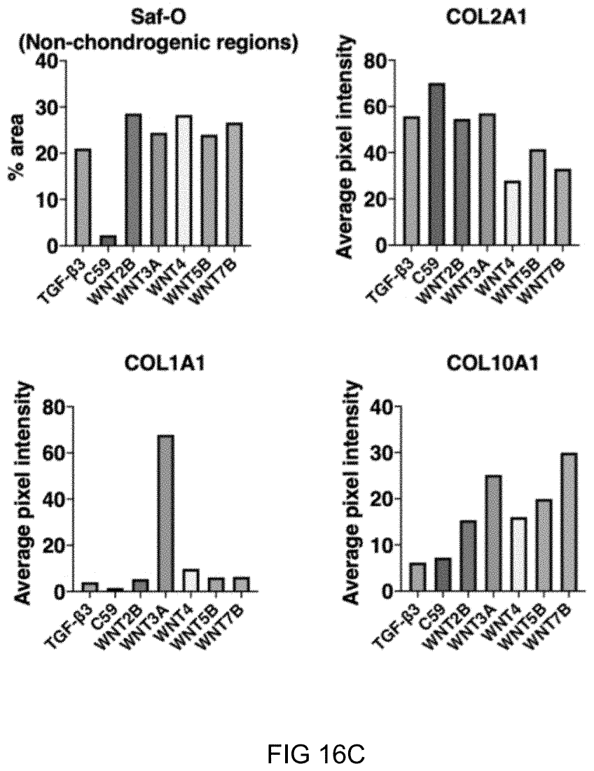

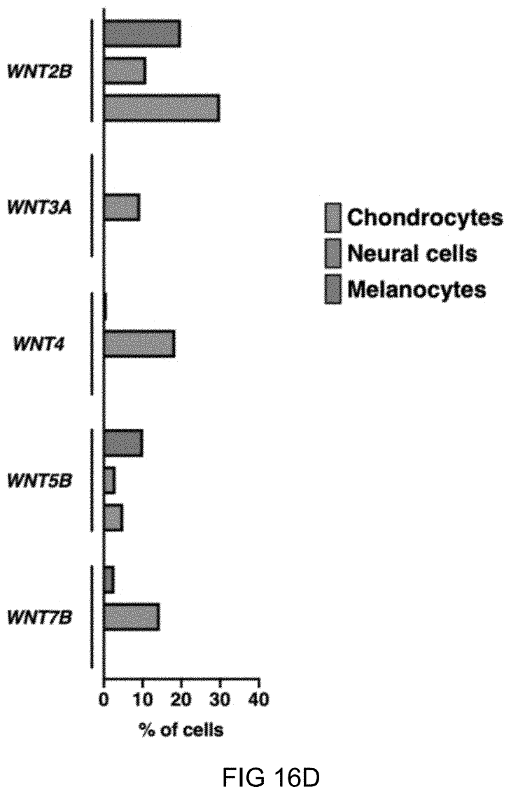

At d28, pellets treated with WNT-C59 exhibited increased expression of ACAN and COMP compared to the standard-treated pellets (FIG. 16C, 16D). Importantly, it was also observed that IFI6 and ISG15, markers for mature-hypertrophic chondrocytes, were downregulated in the WNT-C59-treated pellets, suggesting WNT inhibition may decrease chondrocyte hypertrophy during chondrogenesis.

(ix) WNT Expression with Neurogenesis

To determine the expression patterns of WNTs and to identify the cells responsible for WNT production, WNT expression levels was investigated in multiple cell populations of d14 and d28 pellets (FIG. 6E and FIG. 15E; a total of 5224 d14 cells and a total of 3027 d28 cells analyzed, respectively). In TGF-β3-treated pellets, several canonical WNTs, such as WNT3, WNT3A, and WNT7B, as well as noncanonical WNTs, including WNT4, were enriched in the proliferative population (where the neural cells clustered), while WNT2B and WNT5B could be found in proliferative cells, chondrocytes, and mesenchyme. We did not detect WNT1, WNT2, or WNT8 in any specimens. Upon WNT-C59 treatment, most WNTs showed decreased expression, particularly in proliferative cells. Western blots confirmed that WNT-C59-treated pellets had decreased protein levels of WNT2B, WNT3A, WNT4, and WNT7B (FIG. 6F). Interestingly, WNT-C59 only moderately inhibited WNT5B. Next, these WNT ligands were plotted along with neurogenic and chondrogenic markers in pseudotime to investigate their expression patterns. It was observed that WNT2B, WNT3A, WNT4, and WNT7B clustered with neurogenic markers, whereas WNT5B was upregulated along with chondrogenic differentiation, implying that individual WNTs may play distinct roles in regulating chondrogenesis (FIG. 6G).

### (x) WNTs Alter GAG/DNA and Collagen Production

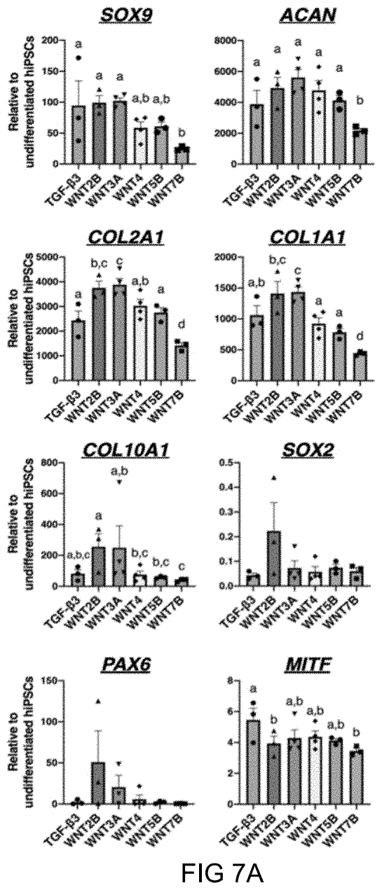

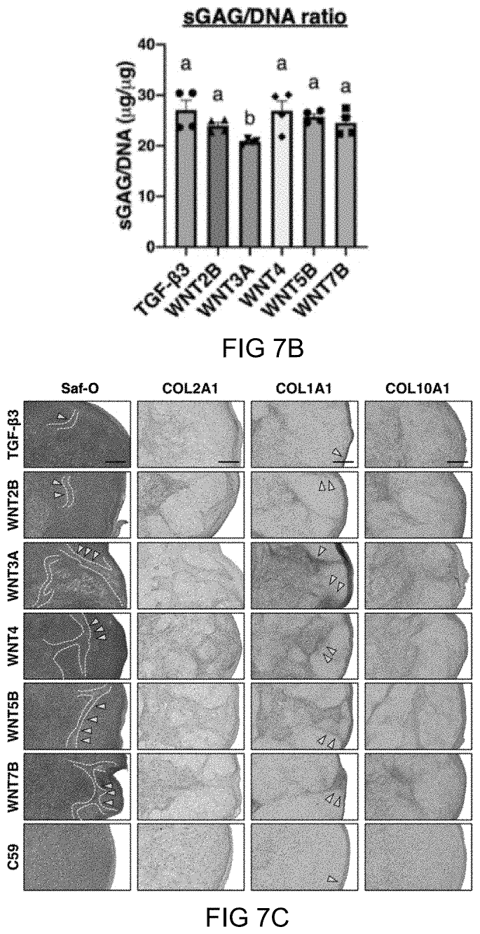

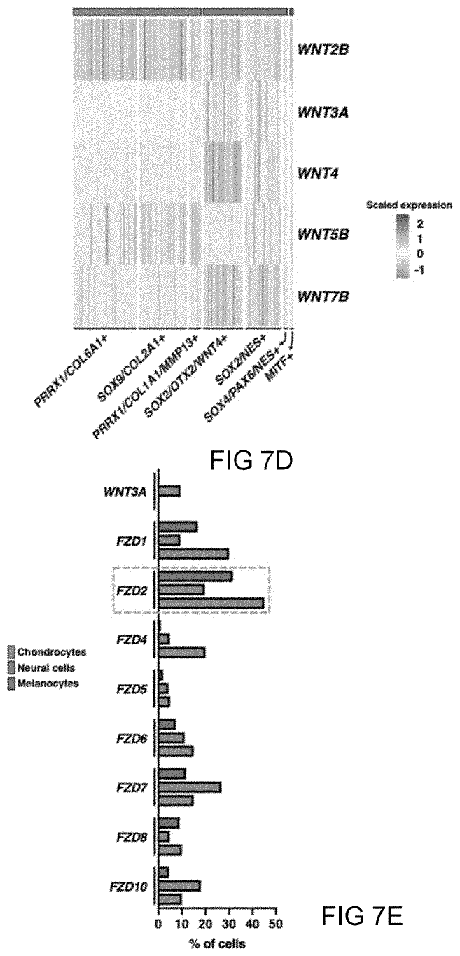

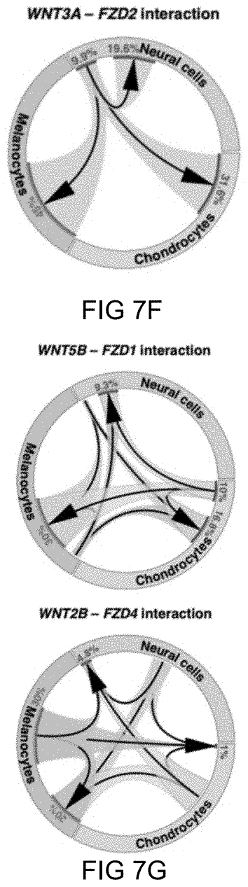

As WNT-C59 is a pan-WNT signaling inhibitor, it, therefore, remained unknown which WNT ligand had the most severe adverse effect on hiPSC chondrogenesis. To answer this question, a variety of WNTs were administrated during pellet culture (FIG. 16A). RT-qPCR analysis showed that only WNT7B significantly decreased chondrogenic markers (SOX9, ACAN, and COL2A1) and osteogenic marker (COL1A1) when compared to TGF-β3 only pellets (FIG. 7A). Interestingly, the pellets treated with WNT2B and WNT3A exhibited increased COL2A1, COL1A1, and COL10A1 expression versus TGF-β3 pellets. However, only the pellets with WNT3A treatment had a significantly decreased GAG/DNA ratio compared to the pellets with TGF-β3 only treatment (FIG. 7B). WNT2B-treated pellets also showed a trend toward the increasing expression of neurogenic markers (PAX6 and SOX2), although not statistically significant. Furthermore, WNT2B- and WNT7B-treated pellets had significantly lower expression of MITF relative to TGF-β3 pellets. It was also observed that WNT ligands may not only regulate their own expression but may also modulate the expression of other WNT ligands (FIG. 16B).

While all pellets had comparable Saf-O staining, WNT treatment increased off-target cells within the pellets (FIG. 7C). Furthermore, these off-target cells exhibited lower production of COL2A1 compared to chondrocytes. Additionally, pellets treated with WNTs, particularly WNT3A, exhibited higher intensity of COL1A1 and COL10A1 staining, which was observed near off-target cells and perichondrium. On the contrary, WNT-C59-treated pellets had low COL1A1 and COL10A1 production, and the staining was mainly at the perichondrium. Together, these results indicate that WNTs increased non-chondrogenic cells and modulated collagen production. The histological images in FIG. 7C were quantified using a published ImageJ protocol (FIG. 16C).

### (xi) Heterocellular WNT Signaling May Regulate Chondrogenesis

To investigate which cell populations are the main sources for the endogenous production of specific WNTs during chondrogenesis, a heatmap in which the expression of WNT ligands against multiple cell populations at the d14 timepoint was plotted (FIG. 7D). It was found that 30% of melanocytes expressed WNT2B, while WNT3A, WNT4, and WNT7B were mainly expressed in neural cells (FIG. 16D). WNT5B was expressed primarily by chondrocytes (about 10% of the chondrocyte population) providing a possible explanation for the upregulation of WNT5B during chondrogenesis. As WNTs are secretory proteins, it was next aimed to identify the potential cell populations receiving WNT signaling based on published lists of ligand-receptor pairs. It was found that 31.6% of chondrocytes expressed FZD2, the highest expression of a WNT receptor in chondrocytes (FIG. 7E). Thus, the multicellular signaling for the WNT3A-FZD2 pair were created and identified that 9.9% of neural cells expressed WNT3A while more than a third of chondrocytes (36.1%) were capable of receiving this ligand (FIG. 7F). In addition, it was also observed that although chondrocytes were the major contributor to WNT5B production, melanocytes (30%) might be the main receiving cell type. Furthermore, while 30% of melanocytes may secrete WNT2B, only 1% of chondrocytes expressed FZD4, one of the main WNT2B receptors (FIG. 7G).

(xii) BMP/GDF Differential Expression after WNT-C59 Treatment

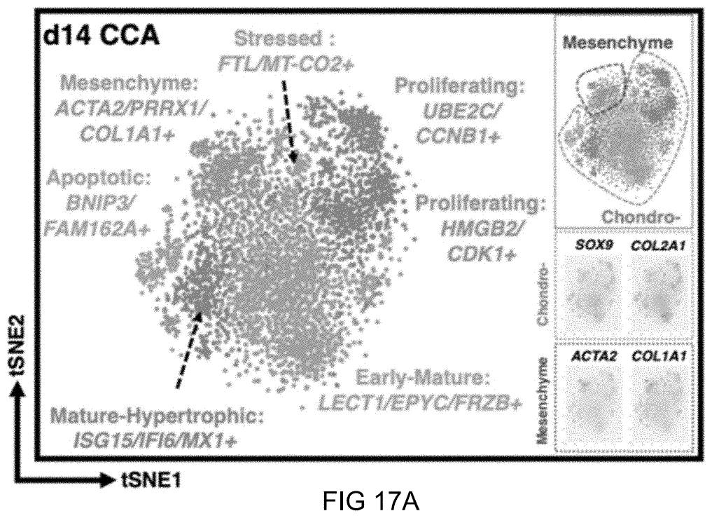

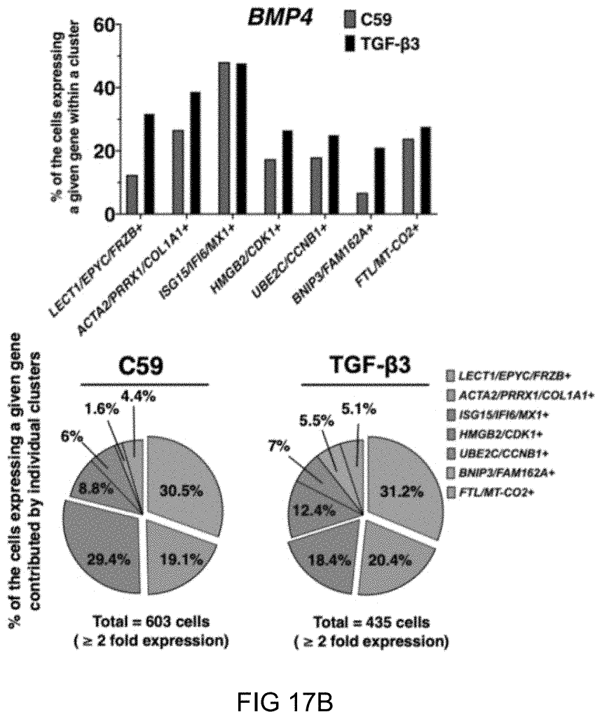

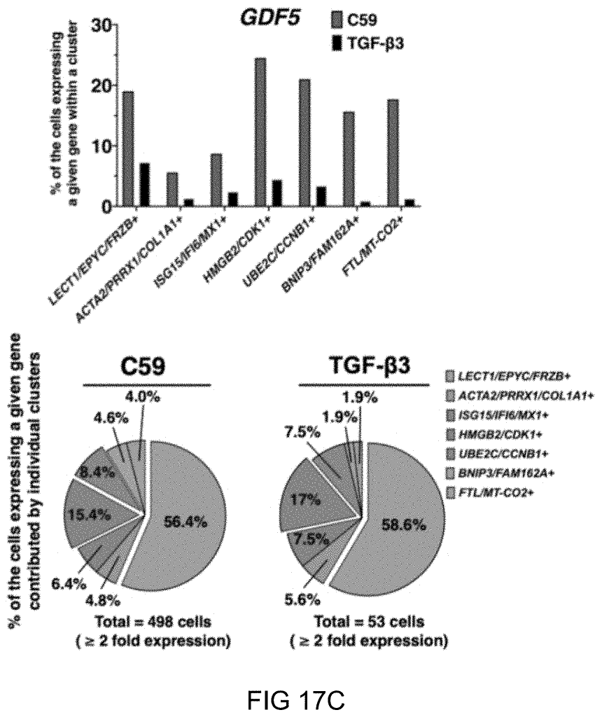

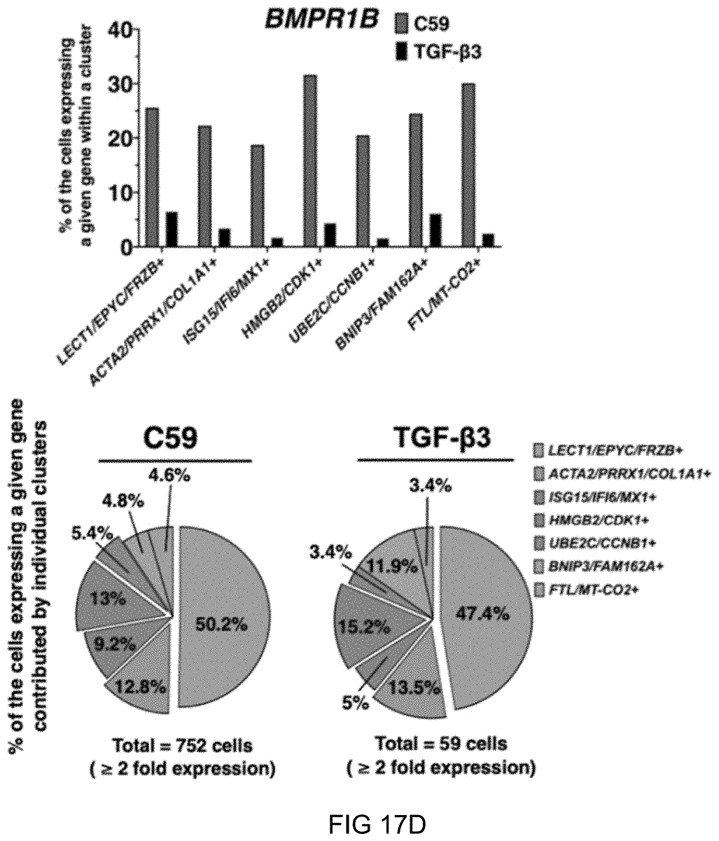

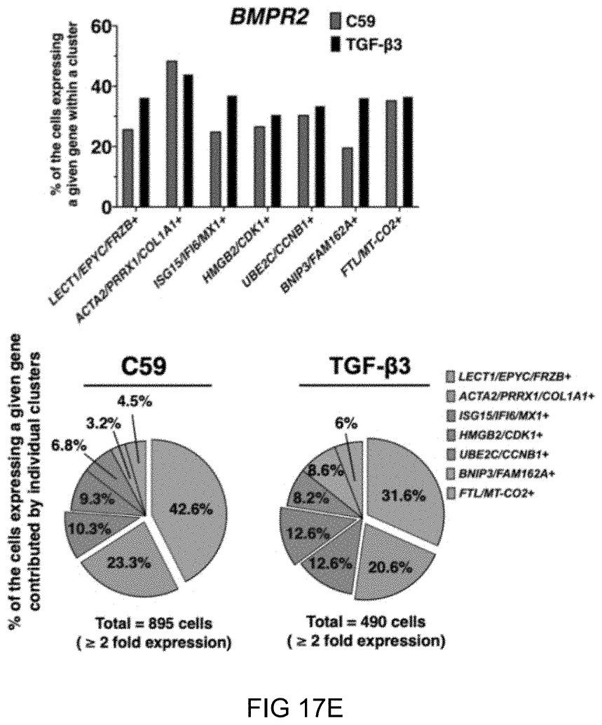

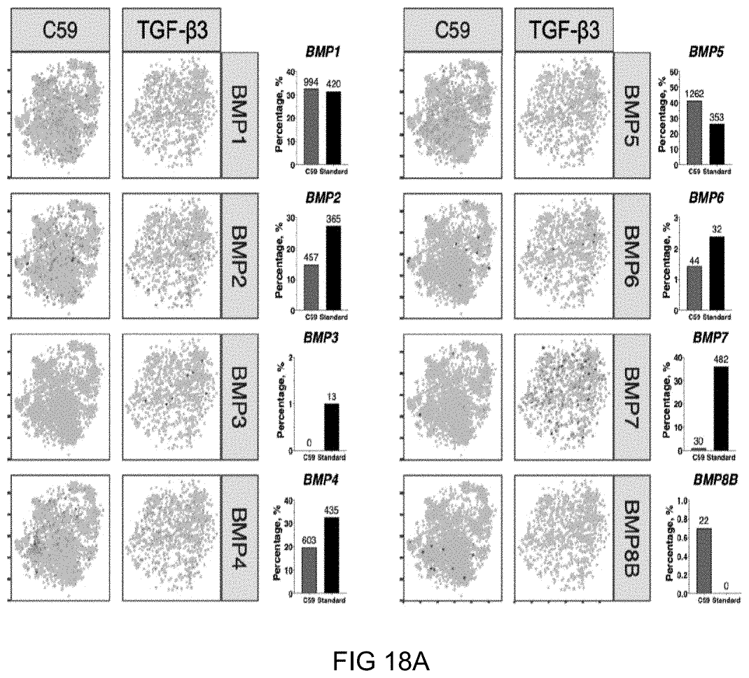

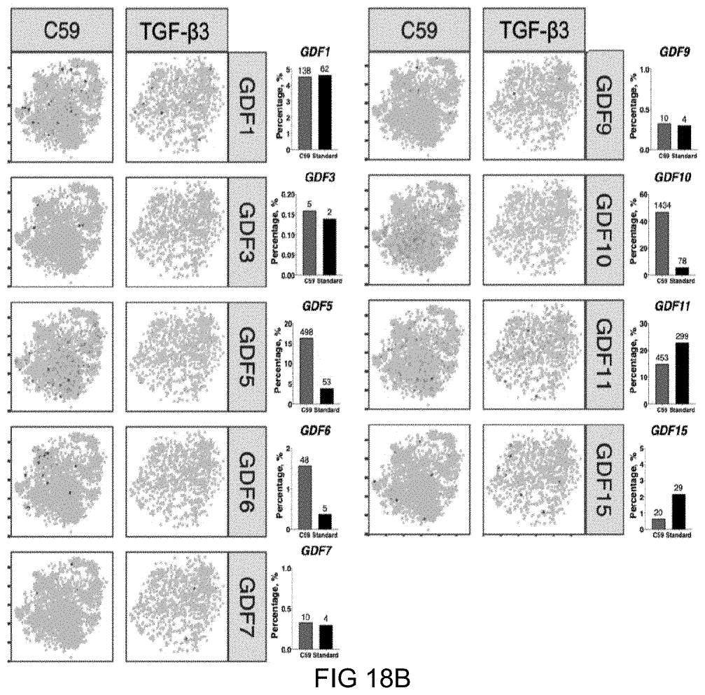

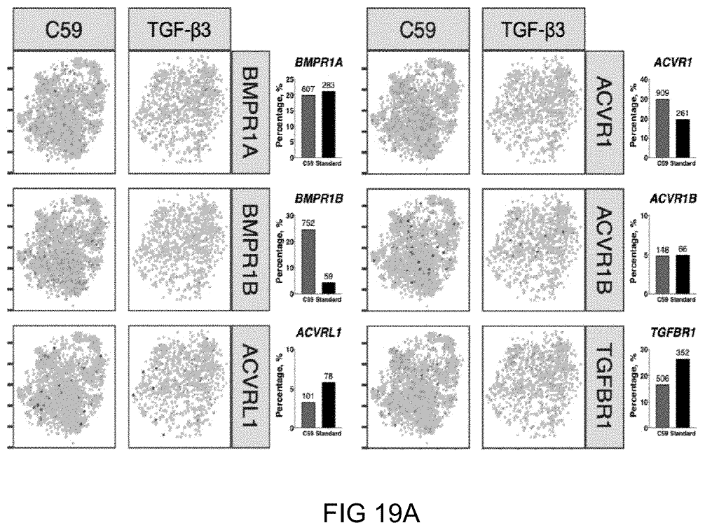

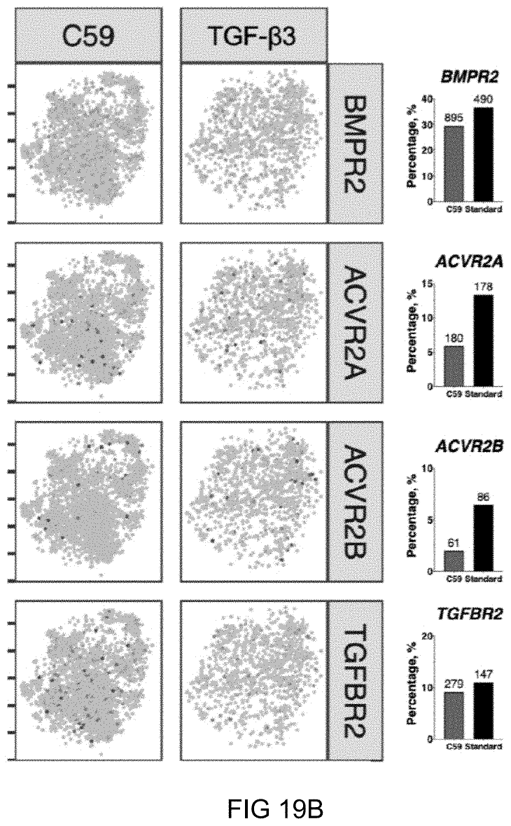

While the precise mechanisms of enhanced chondrogenesis remain to be determined, our CCA analysis showed that six chondrocyte subpopulations and one mesenchymal population were conserved between TGF-β3-treated and WNT-C59-treated d14 pellets: (1) HMGB2/CDK1⁺ proliferating chondrocytes, (2) UBE2C/CCNB1⁺ proliferating chondrocytes, (3) LECT1/EPYC/FRZB⁺ early mature chondrocytes, (4) ISG15/IFI6/MX1⁺ mature-hypertrophic chondrocytes, (5) FTL/MT-CO2⁺ stressed chondrocytes, (6) BNIP3/FAM162A⁺ apoptotic chondrocytes, and ACTA2/PRRX1/COL1A1⁺ mesenchymal cells (FIG. 17A; CCA was performed with a total of 1335 cells from mesenchymal and chondrocyte populations from d14 TGF-β3 pellets and with a total of 3047 cells from mesenchymal and chondrocyte populations from d14 WNT-C59 pellets. It is important to note that off-target cells (i.e., neural cells and melanocytes) were excluded from this analysis). Interestingly, WNT-C59 treatment differentially influenced the expression of various growth factors and receptors in the TGF-β superfamily essential in regulating chondrogenesis (FIG. 17B-17E, FIG. 18A, 18B, and FIG. 19A, 19B).

### Discussion

The therapeutic applications of hiPSCs for cartilage regeneration or disease modeling have been limited by the low-yield of bona fide chondrocytes, accompanied by off-target populations during chondrogenic differentiation. Our GRN analysis revealed two major off-target cell populations, neural cells and melanocytes, which showed high association with WNT4 and WNT2B signaling, respectively. By building heterocellular signaling models, it was shown that off-target cells were the main source of several canonical and noncanonical WNT ligands that were implicated in chondrocyte hypertrophic differentiation. Importantly, inhibition of WNT and MITF, the master regulator of melanocyte development, significantly enhanced homogeneity of hiPSC chondrogenesis by decreasing off-target cells, circumventing the need for prospective sorting and expansion of isolated progenitor cells.

An important finding of this Example was the identification of distinct subtypes of hiPSC-derived chondrocytes, as shown in depth by the comprehensive transcriptomic profiles of each cell type at various differentiation stages. It was also observed that inhibition of WNT signaling during chondrogenesis alters gene expression levels of BMPs/GDFs (e.g., decreasing BMP4 and BMP7 levels) in chondrocytes, which is consistent with a recent study demonstrating decreased BMP activity during MSC chondrogenesis due to WNT inhibition. Another intriguing finding is the discovery of ISG15/IFI6/MX1⁺ mature-hypertrophic chondrocytes as, without scRNA-seq, this unique population has not been reported before. Although the signature genes of this chondrocyte population (e.g., STAT-1) were generally believed to be downstream of IFN-related pathways, IFN expression was not detected. The high expression of IGFBP3 in ISG15/IFI6/MX1⁺ chondrocytes may provide an explanation for this observation, as IGFBP3 can activate STAT-1 expression without the presence of IFN molecules in chondrogenesis. In addition, IGFBP3-enriched chondrocytes also had decreased expression of FOS, essential in driving chondrocytes toward hypertrophy. It has been reported that chondrocyte hypertrophy was largely prevented upon IGFBP3 knockdown in the ATDC5 line. Thus, low FOS expression in ISG15/IFI6/MX1⁺ chondrocytes provides a plausible explanation for their low expression of hypertrophic markers. Nevertheless, the causal relationship between the dual function of IGFBP3 in chondrocyte hypertrophy and WNT inhibition merits further study.

The finding that melanocytes and neural cells were the major off-target cells implies that some, if not all, progenitors may acquire the phenotype of neural crest cells, a transient stem cell population that can give rise to neurons and melanocytes. This differentiation pathway likely occurs at the Cp stage, where we first observed cell populations expressing several markers of neural crest cells. It is likely that the neural crest cells observed in the current Example were also off-target cells (i.e., non-paraxial mesodermal lineage) generated during the early stages of mesodermal differentiation and amplified due to BMP4 treatment at the Cp stage. It has been reported that the Bmp4-Msx1 signaling axis inhibits Wnt antagonists such as Dkk2 and Sfrp2 in dental mesenchyme in mice, implying that BMP4 treatment may promote WNT signaling that is essential for the proliferation of neural crest cells.

Additionally, our sorting results showed that supplementation of WNT increased, but inhibition of WNT decreased, the proportion of CD146/CD166⁺ cells, suggesting that WNT signaling is required to maintain progenitors at the Cp stage. This finding is in agreement with a recent study showing that WNT3A supports the multipotency of hMSCs during in vitro expansion. In our recent publication using a CRISPR-Cas9-edited reporter hiPSC line and scRNA-seq techniques, it was identified that mesenchymal cells triple-positive for CD146, CD166, and PDGFRβ, but negative for CD45, at the Cp stage showed robust chondrogenic potential but little osteogenic capacity compared to unsorted cells, suggesting that CD146/CD166/PDGFRβ⁺ mesenchymal cells may be a unique Cp population. However, whether the CD146/CD166⁺ progenitor population identified in the current example functions like MSCs with multilineage potential warrants future investigation. Furthermore, as distinct subtypes of hiPSC-derived chondrocytes were defined primarily based on marker genes, the complete functionality of these subsets requires future investigation.

Another important contribution of this example is the construction of the GRN of hiPSC chondrogenesis with the presence of minimal off-target cells, ensuring the hub genes identified are truly governing chondrogenic differentiation. In addition to conventional master TFs such as SOX9, several additional hub genes associated with chondrogenesis were also identified. For instance, the expression levels of complement C1q like 1 (C1QL1) were highly correlated with those of COL2A1 in our model. C1QL1 encodes a secreted protein with Ca2⁺ binding sites that regulate synaptogenesis in neuronal cells. However, how C1QL1 affects chondrogenesis or if it plays a role in synovial joint innervation is currently unknown. In addition, our finding of the melanogenic GRN during hiPSC chondrogenesis suggests an off-target cell fate decision in differentiation. This result is further corroborated by the study demonstrating the presence of melanin or lipofuscin on the surface of hiPSC-derived cartilage pellet using rigorous histological staining. Furthermore, it was also revealed the significant association between WNT2B and MITF, providing insights into melanogenesis. Indeed, a recent study proposed genetic variants in WNT2B may serve as a biomarker to predict the survival rate of patients with cutaneous melanoma. It was also identified WNT4 as a hub gene in the GRN of neurogenesis and observed that WNT3A was enriched in the cell populations expressing neural markers. These results are consistent with the previously identified roles for these WNTs in promoting forebrain development.

Heterogenous multicellular signaling models indicate that although most WNTs were produced by off-target cells, these ligands may signal through chondrocytes. It is well recognized that WNT signaling not only blocks SOX9 expression in limb bud mesenchymal cells but also regulates chondrocyte maturation, driving them toward hypertrophy. In agreement with these findings, hiPSC-derived chondrogenic pellets treated with individual WNTs exhibited increased COL10A1 staining. It was also demonstrated that blocking endogenous WNT signaling significantly improved chondrogenesis in hMSCs. These findings reveal the potential modulatory effects of off-target cells on chondrocytes through the WNT signaling pathway, indicating that inhibition of WNT has dual beneficial effects on hiPSC chondrogenesis as it not only removes off-target cells but also prevents chondrocyte hypertrophy.

These findings not only identify the mechanisms regulating the heterogeneity in hiPSC chondrogenesis but, more importantly, provide an enhanced chondrogenic differentiation protocol capable of generating homogenous chondrocytes by removing off-target cells without cell sorting. Furthermore, this protocol has been validated in multiple unique lines, demonstrating its robustness and efficiency in deriving chondrocytes from hiPSCs. a comprehensive map of single-cell transcriptome profiles and GRNs governing cell fate decisions during hiPSC chondrogenesis was also established. These findings provide insights into dynamic regulatory and signaling pathways orchestrating hiPSC chondrogenesis, thereby advancing a further step of cartilage regenerative medicine toward therapeutic applications. This approach also provides a roadmap for the use of single-cell transcriptomic methods for the study and optimization of other in vitro or in vivo differentiation processes.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, a “population” of cells refers to a group of at least 2 cells, e.g. 2 cells, 3 cells, 4 cells, 10 cells, 100 cells, 1000 cells, 10,000 cells, 100,000 cells or any value in between, or more cells. Optionally, a population of cells can be cells which have a common origin, e.g. they can be descended from the same parental cell, they can be clonal, they can be isolated from or descended from cells isolated from the same tissue, or they can be isolated from or descended from cells isolated from the same tissue sample. Preferably, the population of hematopoietic progenitor cells is substantially purified. As used herein, the term “substantially purified” means a population of cells substantially homogeneous for a particular marker or combination of markers. By substantially homogeneous is meant at least 80%, at least 85%, at least 90%, at least 95%, at least 96%, at least 97%, at least 98%, at least 99% or more homogeneous for a particular marker or combination of markers.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method of generating a population of chondrocyte or chondrocyte-like cells, the method comprising:
(i) culturing a population of pluripotent stem cells in a mesoderm differentiation medium to produce a population of chondroprogentior cells; and
(ii) culturing the population of cells obtained from step (i) in a chondrogenic medium comprising a transforming growth factor beta, a WNT signaling inhibitor and optionally bone morphogenic protein and optionally an inhibitor of the microphthalmia-associated transcription factor (MITF) pathway to produce a population of chondrocyte or chondrocyte-like cells.

2. The method of claim 1, wherein the pluripotent stem cells are induced pluripotent stem cells (iPS).

3. The method of claim 1, wherein the pluripotent stem cells are embryonic stem cells.

4. The method of claim 1, wherein the population of chondroprogenitor cells in step (ii) is prepared in a 3D culture.

5. The method of claim 1, wherein the transforming growth factor beta is TGF-β3.

6. The method of claim 1, wherein the WNT signaling inhibitor is WNT-C59.

7. The method of claim 1, wherein the MITF inhibitor is ML329.

8. The method of claim 1, wherein the bone morphogenic protein is BMP-4.

9. (canceled)

10. The method of claim 1, wherein the method does not require prospective sorting or expansion of the isolated population of chondrocyte or chondrocyte-like cells and wherein off-target cells and/or chondrocyte hypertrophy is reduced in the population of chondrocyte or chondrocyte-like cells relative to a population of chondrocyte or chondrocyte-like cells produced by a method which does not inhibit WNT signaling and/or MITF signaling in step (ii).

11. The method of claim 1, wherein the base chondrogenic medium media is a mix of DMEM and F12.

12. The method of claim 1, wherein TGF-β3 is present at a concentration of about 10 ng/ml.

13. The method of claim 1, wherein the WNT-C59 is present at a concentration of about 1 μM.

14. The method of claim 1, wherein the ML329 is present at a concentration of about 1 μM.

15. The method of claim 1, wherein the BMP4 is present at a concentration of about 20 ng/ml.

16. The method of claim 1, wherein the chondroprogenitor cells are cultured in the chondrogenic differentiation medium for about 1 to about 56 days.

17. The method of claim 1, wherein the PS cells are genetically modified.

18. A population of chondrocyte or chondrocyte-like cells, which is produced by a method of claim 1.

19. A method of treating a subject in need thereof, comprising administration of chondrocyte or chondrocyte-like cells produced by the method according to claim 1, wherein the subject has a cartilage-related disease, disorder, or condition.

20. The method of claim 19, wherein the cartilage-related disease, disorder or condition is arthritis, osteoarthritis, rheumatoid arthritis, joint injuries, cartilage defects, or growth-related abnormalities or dysplasias.

21.-26. (canceled)

27. A cell pellet or matrix comprising chondrocyte or chondrocyte-like cells produced according to claim 1, wherein the pellet or matrix comprising the cells has a reduced population off-target cells and have increased heterogeneity compared to chondrocyte-like cells not treated with the Wnt inhibitor or MITF inhibitor.
